(19) United States
(12) Patent Application Publication
(10) Pub. No.: US 2003/0171218 A1
(43) Pub. Date: Sep. 11, 2003

(54) Substituted 3-heteroaryl(amino- or oxy)pyrrolidin-2-ones, their preparation and use as herbicides or plant growth regulators

(76) Inventors: Guido Bojack, Wiesbaden (DE); Lothar Willms, Hofheim (DE); Alfred Angermann, Kriftel/Ts. (DE); Hermann Bieringer, Eppstein (DE); Hubert Menne, Hofheim (DE); Thomas Auler, Bad Soden (DE)

Correspondence Address: William F. Lawrence, Esq., FROMMER, LAWRENCE & HAUG LLP, 745 Fifth Avenue, New York, NY, 10151, US

(21) Appl. No.: 10/191,689

(22) Filed: Jul. 9, 2002

(30) Foreign Application Priority Data
Jul. 11, 2001 (DE) 10135043.0

(51) Int. Cl. C07D 417/02; C07D 413/02; A01N 43/40; A01N 43/72; A01N 43/50; A01N 43/78

(52) U.S. Cl. 504/221; 504/224; 504/248; 504/266; 504/275; 504/270; 544/60; 544/137; 544/139; 544/369; 544/370; 546/269.1; 546/272.7; 546/271.4; 548/182; 548/225; 548/316.4

(57) ABSTRACT

A description is given of substituted 3-heteroaryl(amino- or oxy)pyrrolidin-2-ones of the formula (I), processes for preparing them, and their use as herbicides or as plant growth regulators.

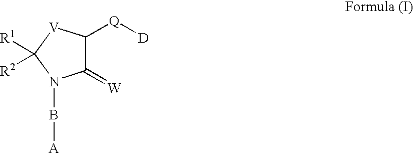

In the formula (I), A is an unsubstituted or substituted aryl; B is a direct bond or a divalent unit; V is, for example, CH₂, S, or O; W is O, S, or H₂; Q is a substituted or unsubstituted bridge; and D is an unsubstituted or substituted C₆-aryl or heteroaryl. R¹ and R² stand for different radicals.

Substituted 3-heteroaryl(amino- or oxy)pyrrolidin-2-ones, their preparation and use as herbicides or plant growth regulators

The invention pertains to the technical field of plant protection compositions, such as herbicides and plant growth regulators, especially herbicides for selectively controlling weed plants in crops.

It is known that 2-amino-4-(N-phenoxyalkylamino)-1,3,5-triazines substituted at least in position 6 possess herbicidal and growth regulating properties; cf. DE 19828519. Moreover, it is known from WO9533719 and WO9637466 that certain pyrrolidones, thiazolidones, and oxazolidones possess herbicidal properties.

The known active substances have disadvantages in some cases in the course of their use, such as (a) inadequate herbicidal activity against weed plants, or (b) too narrow a spectrum of weed plants that can be controlled with an active substance, or (c) inadequate selectivity in crops. Other active substances cannot be produced economically on an industrial scale, owing to reagents and precursors that are difficult to obtain, or possess inadequate chemical stabilities.

It is an object of the invention to provide chemical compounds which can be used, where appropriate, with advantages as herbicides or plant growth regulators.

The present invention provides compounds of the formula (I) and salts thereof

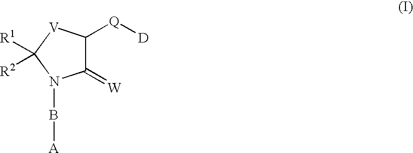

in which

A is a radical as defined under (a1) to (a5), where

(a1) is unsubstituted aryl having 6-18 carbon atoms, preferably a monocyclic, bicyclic or tricyclic aromatic system comprising five- or six-membered carbocyclic rings, or unsubstituted heterocyclyl having from 2 to 14 carbon atoms, preferably a monocyclic, bicyclic or tricyclic aromatic, partially unsaturated or saturated heterocyclic system based on five- or six-membered carbocyclic rings, at least one ring containing one or more heteroatoms, preferably 1-4 ring heteroatoms, especially from 1 to 3 identical or different ring heteroatoms from the group N, O, and S,

(a2) is a radical as defined under (al) further substituted by one or more fused-on nonaromatic carbocyclic rings having in each case from 4 to 7 ring atoms or heterocyclic rings having in each case from 4 to 7 ring atoms and one or more ring heteroatoms, preferably having from 1 to 4 ring heteroatoms, in particular having from 1 to 3 ring heteroatoms from the group N, O, and S,

(a3) is a radical as defined under (al) or (a2) which is further substituted, preferably by one or more identical or different radicals from the group consisting of amino, hydroxyl, halogen, preferably fluorine and chlorine, nitro, cyano, mercapto, carboxyl, carbamoyl, SF₅, aminosulfonyl, (C₁-C₆)-alkyl, (C₃-C₉)-cycloalkyl, (C₂-C₆)-alkenyl, (C₅-C₉)-cycloalkenyl, (C₂-C₆)-alkynyl, mono-((C₁-C₆)-alkyl)-amino, di-((C₁-C₆)-alkyl-amino, N-((C₁-C₆)-alkanoyl)-amino, (C₁-C₆)-alkoxy, (C₂-C₆)-alkenyloxy, (C₂-C₆)-alkynyloxy, (C₃-C₉)-cycloalkoxy, (C₅-C₉)-cycloalkenyloxy, ((C₁-C₆)-alkoxy)-carbonyl, ((C₂-C₆)-alkenyloxy)-carbonyl, ((C₂-C₆)-alkynyloxy)-carbonyl, aryloxycarbonyl, (C₁-C₆)-alkanoyl, ((C₂-C₆)-alkenyl)-carbonyl, ((C₂-C₆)-alkynyl)-carbonyl, arylcarbonyl, (C₁-C₆)-alkylthio, (C₃-C₉)-cycloalkylthio, (C₂-C₆)-alkenylthio, (C₅-C₉)-cycloalkenylthio, (C₂-C₆)-alkynylthio, (C₁-C₆)-alkynylsulfinyl, (C₁-C₆)-alkylsulfonyl, mono-((C₁-C₆)-alkyl)-aminosulfonyl, di-((C₁-C₆)-alkyl)-aminosulfonyl, N-((C₁-C₆)-alkyl)-aminocarbonyl, N,N-di-((C₁-C₆)-alkyl)-aminocarbonyl, N-((C₁-C₆)-alkanoyl)-aminocarbonyl, N-((C₁-C₆)-alkanoyl)-N-((C₁-C₆)-alkyl)-aminocarbonyl, aryl, aryloxy, benzyl, benzyloxy, benzylthio, arylthio, thiocyanato, tri-((C₁-C₆)-alkyl)-silyl, (C₁-C₆)-alkanoyloxy, (C₁-C₆)-alkylsulfonyloxy, ((C₁-C₆)-alkoxy)-carbonyloxy, ((C₂-C₆)-alkenyloxy)-carbonyloxy, ((C₂-C₆)-alkynyloxy)-carbonyloxy, N-((C₁-C₆)-alkyl)-aminocarbonyloxy, N,N-d 1-((C₁-C₆)-alkyl)-amino-carbonyloxy, each of the above hydrocarbon substituents (a3) being unsubstituted or substituted by one or more identical or different radicals from the group consisting of amino, hydroxyl, halogen, nitro, cyano, mercapto, carboxyl, carbamoyl, aminosulfonyl, (C₃-C₆)-cycloalkyl, (C₅-C₉)-cycloalkenyl, mono-((C₁-C₄)-al kyl)-amino, di-((C₁-C₄)-alkyl)-amino, N-((C₁-C₆)-alkanoyl)-amino, N-((C₁-C₆)-alkanoyl)-N-((C₁-C₄)-alkyl)-amino, (C₁-C₆)-alkoxy, (C₁-C₆)-alkoxy-(C₁-C₆)-alkoxy, (C₁-C₆)-alkoxy-[poly-(C₁-C₄)-alkylenoxy)], hydroxy-(C₁-C₆)-alkoxy, hydroxy-[poly-(C₁-C₄)-alkylenoxy)], (C₃-C₆)-cycloalkoxy, (C₁-C₆)-haloalkoxy, (C₂-C₆)-alkenyloxy, (C₅-C₉)-cycloalkenyloxy, (C₂-C₆)-haloalkenyloxy, (C₂-C₆)-alkynyloxy, (C₂-C₆)-haloalkynyloxy, (C₁-C₄)-alkoxycarbonyl, aryloxycarbonyl, aryloxycarbonyl, (C₁-C₆)-alkanoyl, arylcarbonyl, (C₁-C₄)-alkylthio, (C₃-C₆)-cycloalkylthio, (C₂-C₄)-alkenylthio, (C₅-C₉)-cycloalkenylthio, (C₂-C₄)-alkynylthio, (C₁-C₄)-alkylsulfinyl, (C₁-C₄)-alkylsulfonyl, mono-((C₁-C₄)-alkyl)-aminosulfonyl, di-((C₁-C₄)-alkyl)-aminosulfonyl, N-((C₁-C₄)-alkyl)-aminocarbonyl, N,N-di-((C₁-C₄)-alkyl)-aminocarbonyl, N-((C₁-C₄)-alkanoyl)-aminocarbonyl and N-((C₁-C₄)-alkanoyl)-N-((C₁-C₄)-alkyl)-aminocarbonyl and, in the case of cyclic radicals, additionally (C₁-C₄)-alkyl, (C₁-C₄)-haloalkyl, (C₂-C₄)-alkenyl, (C₂-C₄)-alkynyl, (C₂-C₆)-haloalkenyl, (C₂-C₆)-haloalkynyl, (C₂-C₄)-alkoxy-(C₁-C₄)-alkyl, ((C₁-C₄)-alkoxy)-carbonyl-(C₁-C₄)-alkyl, ((C₁-C₄)-alkylthio)-(C₁-C₄)-alkyl, mono-(C₁-C₄)-alkylamino-(C₁-C₄)-alkyl and di-(C₁-C₄)-alkyl)-amino-(C₁-C₄)-alkyl,

(a4) is (Q₁-C₆)-alkyl, (C₃-C₉)-cycloalkyl, (C₂-C₆)-alkenyl, (C₅-C₉)-cycloalkenyl or (C₂-C₆)-alkynyl,

(a5) is one of the hydrocarbon radicals defined under (a4) further carrying one or more identical or different radicals from the group of the radicals of subgroups (a5.1) and (a5.2), where subgroup (a5.1) is composed of the radicals halogen, preferably fluorine, amino, hydroxyl, nitro, cyano, mercapto, carboxyl, carbamoyl, SF₅, aminosulfonyl, (C₃-C₉)-cycloalkyl, (C₅-C₉)-cycloalkenyl, mono-(C₁-C₆)-alkylamino, di-((C₁-C₆)-alkyl)-amino, N-((C₁-C₆)-alkanoyl)-amino, (C₁-C₆)-alkoxy, (C₂-C₆)-alkenyloxy, (C₂-C₆)-alkynyloxy, (C₃-C₉)-cycloalkoxy, (C₅-C₉)-cycloalkenyloxy, ((C₁-C₆)-alkoxy)-carbonyl, (C₁-C₆)-alkanoyl, (C₁-C₆)-alkylthio, (C₃-C₉)-cycloalkylthio, (C₂-C₆)-alkenylthio, (C₅-C₉)-cycloalkenylthio, (C₂-C₆)-alkynylthio, (C₁-C₆)-alkynylsulfinyl, (C₁-C₆)-alkylsulfonyl, mono-((C₁-C₆)-alkyl)-aminosulfonyl, di-((C₁-C₆)-alkyl)-aminosulfonyl, N-((C₁-C₆)-alkyl)-aminocarbonyl, N,N-di-((C₁-C₆)-alkyl)-aminocarbonyl, N-((C₁-C₆)-alkanoyl)-aminocarbonyl, N-((C₁-C₆)-alkanoyl)-N-((C₁-C₆)-alkyl)-aminocarbonyl and, in the case of cyclic radicals, additionally (C₁-C₄)-alkyl, (C₂-C₆)-alkenyl, (C₂-C₆)-alkynyl, (C₁-C₄)-alkoxy-(C₁-C₄)-alkyl, ((C₁-C₄)-alkoxy)-carbonyl)-(C₁-C₄)-alkyl, (C₁-C₄)-alkylthio-(C₁-C₄)-alkyl, mono-((C₁-C₄)-alkyl)-amino-(C₁-C₄)-alkyl and di-((C₁-C₄)-alkyl)-amino-(C₁-C₄)-alkyl, each of the above hydrocarbon substituents (a5.1) being unsubstituted or substituted by one or more identical or different radicals from the group consisting of halogen, nitro, cyano, thiocyanato, (C₁-C₄)-alkoxy, (C₁-C₄)-haloalkoxy and (C₁-C₄)-alkylthio and, in the case of cyclic radicals, additionally (C₁-C₄)-alkyl and halo-(C₁-C₄)-alkyl, and subgroup (a5.2) is composed of the radicals aryl, aryloxy, arylthio, heteroaryloxy and heteroarylthio, each of the last-mentioned 6 radicals being unsubstituted or substituted by one or more identical or different radicals from the group [=substituent group (a5.2.1)] consisting of halogen, amino, hydroxyl, nitro, cyano, mercapto, carboxyl, carbamoyl, aminosulfonyl, (C₁-C₄)-alkyl, (C₁-C₄)-haloalkyl, ((C₁-C₄)-alkoxy)-carbonyl-(C₁-C₄)-alkyl, mono-((C₁-C₄)-alkyl)-amino-(C₁-C₄)-alkyl and di-((C₁-C₄)-alkyl)-amino-(C₁-C₄)-alkyl, (C₂-C₆)-cycloalkyl, (C₂-C₄)-alkenyl, (C₅-C₉)-cycloalkenyl, (C₂-C₆)-alkynyl, mono-((C₁-C₄)-alkyl)-amino, di-((C₁-C₄)-alkyl)-amino, N-((C₁-C₆)-alkanoyl)-amino, N-((C₁-C₆)-alkanoyl)-amino, N-((C₁-C₆)-alkanoyl)-N-((C₁-C₄)-alkyl)-amino, (C₁-C₆)-alkoxy, (C₁-C₆)-alkoxy-[poly-(C₁-C₄)-alkylenoxy)]-, hydroxy-[poly-(C₁-C₄)-alkylenoxy)]-(C₃-C₆)-cycloalkylenoxy, (C₂-C₆)-alkenyloxy, (C₂-C₆)-alkynyloxy, ((C₁-C₄)-alkoxy)-carbonyl, (C₁-C₆)-alkanoyl, (C₁-C₄)-alkylthio, (C₃-C₆)-cycloalkylthio, (C₂-C₄)-alkenylthio, (C₅-C₉)-cycloalkenylthio, (C₂-C₄)-alkynylthio, (C₁-C₄)-alkylsulfinyl, (C₁-C₄)-alkylsulfonyl, mono-(C₁-C₄)-alkylaminosulfonyl, di-((C₁-C₄)-alkyl)-aminosulfonyl, N-((C₁-C₄)-alkyl)-aminocarbonyl, N,N-di-((C₁-C₄)-alkyl)-aminocarbonyl, N-((C₁-C₄)-alkanoyl)-aminocarbonyl and N-((C₁-C₄)-alkanoyl)-N-((C₁-C₄)-alkyl)-aminocarbonyl, each of the hydrocarbon substituents from the above substituent subgroup (a5.2.1) being unsubstituted or substituted by one or more identical or different radicals from the group consisting of halogen, nitro, cyano, thiocyanato, (C₁-C₄)-alkoxy, (C₁-C₄)-haloalkoxy and (C₁-C₄)-alkylthio and, in the case of cyclic radicals, additionally (C₁-C₄)-alkyl and halo-(C₁-C₄)-alkyl,

B is a direct bond or (C₁-C₄)-alkylene, (C₂-C₄)-alkenylene, (C₂-C₄)-alkynylene, in which individual carbon atoms may have been replaced by nitrogen atoms, preferably a group of the formula —CH₂—, —CH₂CH₂—, —CH(CH₃)—, —C(CH₃)₂—, —CH═CH—, —C≡C—, —N═CH—, —NH—CH₂—, each of said divalent radicals being unsubstituted or substituted by one or more radicals from the group consisting of halogen, amino and cyano, and is preferably a direct bond,

W is O, S or H₂, preferably O,

V is a divalent group of the formula CH₂, S, O, or CHR³ or CR³R⁴, where

R³ is selected from the group consisting of

(C₁-C₆)-alkyl, (C₂-C₆)-alkenyl, (C₂-C₆)-alkynyl, (C₃-C₆)-cycloalkyl, (C₃-C₆)-cycloalkenyl, mono-(C₁-C₆)-alkylamino, di-((C₁-C₆)-alkyl)-amino, N-(C₁-C₆)-alkanoyl)-amino, (C₁-C₆)-alkoxy, (C₂-C₆)-alkenyloxy, (C₂-C₆)-alkynyloxy, (C₃-C₉)-cycloalkoxy, (C₅-C₉)-cycloalkenyloxy, (C₁-C₆)-alkylthio, (C₃-C₉)-cycloalkylthio, (C₂-C₆)-alkenylthio, (C₅-C₉)-cycloalkenylthio, (C₂-C₆)-alkynylthio, (C₁-C₆)-alkanoyl, ((C₂-C₆)-alkenyl)-carbonyl, (C₂-C₆)-(alkynyl)-carbonyl, arylcarbonyl, ((C₁-C₆)-alkoxy)-carbonyl, ((C₂-C₆)-alkenyloxy)-carbonyl, ((C₂-C₆)-alkynoxy)-carbonyl, aryloxycarbonyl, (C₁-C₆)-alkylsulfinyl and (C₁-C₆)-alkylsulfonyl, each of the above radicals indicated for R³ being unsubstituted or substituted by halogen or cyano, and

R⁴ independently of R³ being one of the radicals defined under R³,

R¹ and R² independently of one another are each H, amino, (C₁-C₆)-alkyl, ((C₂-C₆)-alkenyl)-carbonyl, ((C₂-C₆)-alkynyl)-carbonyl, mono-(C₁-C₆)-alkyl-amino, (C₃-C₆)-cycloalkyl, (C₁-C₆)-alkoxy, (C₃-C₆)-cycloalkoxy, (C₁-C₆)-alkanoyl, ((C₁-C₆)-alkenyl)-carbonyl, ((C₂-C₆)-alkynyl)-carbonyl, ((C₁-C₆)-alkoxy)-carbonyl, di-((C₁-C₆)-alkyl)-amino-(C₁-C₆)-alkyl, di-((C₁-C₆)-alkyl)-amino-(C₁-C₆)-alkenyl, di-((C₁-C₆)-alkyl)-aminocarbonyl, each of the last-mentioned 14 radicals being unsubstituted or substituted by one or more radicals from the group consisting of halogen and cyano, and are preferably H or methyl,

Q is a divalent group of the formula O, S, SO, SO₂, NR⁵, CR⁶R⁷, CR⁶OR⁷, CO, CS or C═N—R′, in which R′ is H, OH, (C₁-C₄)-alkyl or (C₁-C₄)-alkoxy, and in the formulae

R⁶ and R⁷ independently of one another are hydrogen, (C₁-C₄)-alkyl, (C₁-C₄)-alkenyl, (C₃-C₆)-cycloalkyl, (C₃-C₆)-cycloalkenyl or cyano and R⁵ is hydrogen, hydroxyl, formyl, NR⁸R⁹, (C₁-C₄)-alkyl, (C₂-C₄)-alkenyl, (C₂-C₄)-alkynyl, (C₃-C₆)-cycloalkyl, (C₃-C₆)-cycloalkenyl, (C₁-C₄)-alkyloxy, (C₂-C₄)-alkenyloxy or (C₂-C₄)-alkynyloxy, each of the last-mentioned hydrocarbon radicals for R⁵ being unsubstituted or substituted by one or more substituents from the group consisting of OR⁸, COR″, COOR⁸, OCOR⁸, CN, halogen, S(O)ₚR⁸ [where p=0, 1, or 2], NR³R⁹, NO₂, NR⁸COR⁹, NR⁸CONR⁹R¹⁰, CONR⁸R⁹ and heterocyclyl, and each of the radicals R⁸, R⁹ and R¹⁰ independently of one another being hydrogen, (C₁-C₄)-alkyl, (C₂-C₄)-alkenyl or (C₂-C₄)-alkynyl, each of the last-mentioned 3 radicals being unsubstituted or substituted by halogen, and

D is aryl if Q is selected from the group O, S, SO, SO₂ or NR⁵, or is heteroaryl having 3-5 carbon atoms and 1, 2, or 3 identical or different ring heteroatoms selected from the group N, O, and S, if Q is selected from the group O, S, SO, SO₂, NR⁵, CR⁶R⁷, CR⁶OR⁷, CO, CS or C═N—R′, the aryl or the heteroaryl being unsubstituted or substituted, preferably unsubstituted or substituted by one or more identical or different radicals from the group consisting of amino, hydroxyl, halogen, preferably fluorine and chlorine, nitro, cyano, mercapto, thiocyanato, carboxyl, carbamoyl, SF₅, methylsulfonyl, aminosulfonyl, (C₁-C₆)-alkyl, (C₃-C₉)-cycloalkyl, (C₂-C₆)-alkenyl, (C₅-C₉)-cycloalkenyl, (C₂-C₆)-alkynyl, mono-((C₁-C₆)-alkyl)-amino, di-((C₁-C₆)-alkyl)-amino, ((C₃-C₆)-cycloalkyl)-amino, N-((C₁-C₆)-alkanoyl)-amino, (C₁-C₆)-alkoxy, (C₂-C₆)-alkenyloxy, (C₂-C₆)-alkynyloxy, (C₃-C₉)-cycloalkoxy, (C₅-C₉)-cycloalkenyloxy, ((C₁-C₆)-alkoxy)-carbonyl, ((C₂-C₆)-alkenyloxy)-carbonyl, ((C₂-C₆)-alkynyloxy)-carbonyl, aryloxycarbonyl, (C₁-C₆)-alkanoyl, ((C₂-C₆)-alkenyl)-carbonyl, ((C₂-C₆)-alkynyl)-carbonyl, arylcarbonyl, (C₁-C₆)-alkylthio, (C₃-C₉)-cycloalkylthio, (C₂-C₆)-alkenylthio, (C₅-C₉)-cycloalkenylthio, (C₂-C₆)-alkynylthio, (C₁-C₆)-alkylsulfinyl, (C₁-C₆)-alkylsulfonyl, mono-((C₁-C₆)-alkyl)-aminosulfonyl, d 1-((C₁-C₆)-alkyl)-aminosulfonyl, N-((C₁-C₆)-alkyl)-aminocarbonyl, N,N-di-((C₁-C₆)-alkyl)-aminocarbonyl, N-((C₁-C₆)-alkanoyl)-aminocarbonyl, N-((C₁-C₆)-alkanoyl)-N-((C₁-C₆)-alkyl)-aminocarbonyl, aryl, aryloxy, benzyl, benzyloxy, benzylthio, arylthio, tri-((C₁-C₆)-alkyl)-silyl, (C₁-C₆)-alkanoyloxy, (C₁-C₆)-alkylsulfonyloxy, ((C₁-C₆)-alkoxy)-carbonyloxy, ((C₂-C₆)-alkenyloxy)-carbonyloxy, ((C₂-C₆)-alkynyloxy)-carbonyloxy, N-((C₁-C₆)-alkyl)-aminocarbonyloxy and N,N,-di-((C₁-C₆)-alkyl)-aminocarbonyloxy, each of the above hydrocarbon substituents being unsubstituted or substituted by one or more identical or different radicals from the group consisting of amino, hydroxyl, halogen, nitro, cyano, mercapto, carboxyl, carbamoyl, aminosulfonyl, (C₃-C₆)-cycloalkyl, (C₂-C₄)-alkenyl, (C₅-C₉)-cycloalkenyl, (C₂-C₆)-alkynyl, mono-(C₁-C₄)-alkylamino, di-((C₁-C₄)-alkyl)-amino, N-((C₁-C₆)-alkanoyl)-amino, N-((C₁-C₆)-alkanoyl)-N-((C₁-C₄)-alkyl)-amino, (C₁-C₆)-alkoxy, (C₁-C₆)-alkoxy-(C₁-C₆)-alkoxy, (C₁-C₆)-alkoxy-[poly-(C₁-C₄)-alkylenoxy)]-, hydroxy-(C₁-C₆)-alkoxy, hydroxy-[poly-(C₁-C₄)-alkylenoxy)]-, (C₃-C₆)-cycloalkoxy, (C₁-C₆)-haloalkoxy, (C₂-C₆)-alkenyloxy, (C₅-C₉)-cycloalkenyloxy, (C₂-C₆)-haloalkenyloxy, (C₂-C₆)-alkynyloxy, (C₂-C₆)-haloalkynyloxy, ((C₁-C₄)-alkoxy)-carbonyl, aryloxycarbonyl, aryloxycarbonyl, (C₁-C₆)-alkanoyl, arylcarbonyl, (C₁-C₄)-alkylthio, (C₃-C₆)-cycloalkthio, (C₂-C₄)-alkenylthio, (C₅-C₉)-cycloalkenylthio, (C₂-C₄)-alkynylthio, (C₁-C₄)-alkylsulfinyl, (C₁-C₄)-alkylsulfonyl, mono-((C₁-C₄)-alkyl)-aminosulfonyl, di-((C₁-C₄)-alkyl)-aminosulfonyl, N-((C₁-C₄)-alkyl)-aminocarbonyl, N,N,-di-((C₁-C₄)-alkyl)-aminocarbonyl, N-((C₁-C₄)-alkanoyl)-aminocarbonyl and N-((C₁-C₄)-alkanoyl)-N-((C₁-C₄)-alkyl)-aminocarbonyl and, in the case of cyclic radicals, additionally (C₁-C₄)-alkyl, (C₁-C₄)-haloalkyl, (C₂-C₄)-alkenyl, (C₂-C₄)-alkynyl, (C₂-C₆)-haloalkenyl, (C₂-C₆)-haloalkynyl, (C₂-C₄)-alkoxy)-(C₁-C₄)-alkyl, (C₁-C₄)-alkoxycarbonyl-(C₁-C₄)-alkyl, (C₁-C₄)-alkylthio-(C₁-C₄)-alkyl, mono-((C₁-C₄)-alkyl)-amino-(C₁-C₄)-alkyl and di-((C₁-C₄)-alkyl)-amino-(C₁-C₄)-alkyl.

The compounds of the formula (I) can form salts by addition of a suitable inorganic or organic acid, such as, for example, HCl, HBr, H₂SO₄ or HNO₃, but also oxalic acid or sulfonic acids, to a basic group, such as, for example, amino or alkylamino. Suitable substituents which are present in deprotonated form, such as, for example, sulfonic acids or carboxylic acids, can form inner salts with groups which for their part can be protonated, such as amino groups. Salts can also be formed by replacing the hydrogen of suitable substituents, such as, for example, sulfonic acids or carboxylic acids, by an agriculturally suitable cation. These salts are, for example, metal salts, in particular alkali metal salts or alkaline earth metal salts, in particular sodium salts and potassium salts, or else ammonium salts, salts with organic amines or quaternary ammonium salts with cations of the formula [NRR′R″R′″]⁺, in which R to R′″ independently of one another are each an organic radical, in particular alkyl, aryl, arylalkyl or alkylaryl.

Hereinbelow, compounds of the formula (I) according to the invention and their salts are also abbreviated to “compounds (I) according to the invention.”

In formula (I) and all the formulae hereinbelow, the radicals alkyl, alkoxy, haloalkyl, haloalkoxy, alkylamino and alkylthio and the corresponding unsaturated and/or substituted radicals can in each case be straight-chain or branched in the carbon skeleton. Unless specifically mentioned otherwise, the lower carbon skeletons, for example with 1 to 6 carbon atoms, in particular 1 to 4 carbon atoms, or in the case of unsaturated groups with 2 to 6 carbon atoms, in particular 2 to 4 carbon atoms, are preferred for these radicals. Alkyl radicals, also in the composite meanings, such as alkoxy, haloalkyl, and the like, are, for example, methyl, ethyl, n- or isopropyl, n-, iso-, t- or 2-butyl, pentyls, hexyls, such as n-hexyl, isohexyl and 1,3-dimethylbutyl, heptyls, such as n-heptyl, 1-methylhexyl and 1,4-dimethylpentyl; alkenyl and alkynyl radicals have the meaning of the possible unsaturated radicals which correspond to the alkyl radicals and contain at least one double bond and triple bond, respectively, preferably one double bond and triple bond, respectively. Alkenyl is, for example, allyl, 1-methylprop-2-en-1-yl, 2-methylprop-2-en-1-yl, but-2-en-1-yl, but-3-en-1-yl, 1-methyl-but-3-en-1-yl and 1-methyl-but-2-en-1-yl; alkynyl is, for example, propargyl, but-2-yn-1-yl, but-3-yn-1-yl, 1-methylbut-3-yn-1-yl. Cycloalkyl is a carbocyclic saturated ring system having preferably 3-8 carbon atoms, preferably 3 to 6 carbon atoms, for- example cyclopropyl, cyclobutyl, cyclopentyl or cyclohexyl. In the case of substituted cycloalkyl, this includes cyclic systems with substituents, where the substitutents are attached to the cycloalkyl radical via a double bond, for example an alkylidene group such as methylidene. Substituted cycloalkyl also includes polycyclic aliphatic systems, such as, for example, bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, adamantan-1-yl and adamantan-2-yl. Cycloalkenyl is a carbocyclic non-aromatic, partially unsaturated ring system having preferably 4-8 carbon atoms, in particular 5 to 7 carbon atoms, for example 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl. For substituted cycloalkenyl, the illustrations for substituted cycloalkyl apply correspondingly.

Halogen is, for example, fluorine, chlorine, bromine or iodine. Haloalkyl, -alkenyl and -alkynyl is alkyl, alkenyl and alkynyl, respectively, which is partially or fully substituted by halogen, preferably by fluorine, chlorine and/or bromine, in particular by fluorine or chlorine, for example, monohaloalkyl, perhaloalkyl, CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; this applies correspondingly to haloalkenyl and other halogen-substituted radicals.

Aryl is a mono-, bi- or polycyclic aromatic system, for example phenyl, naphthyl, tetrahydronaphthyl, indenyl, indanyl, pentalenyl, fluorenyl and the like, preferably phenyl.

A heterocyclic radical or ring (heterocyclyl or heteroaryl) can be saturated, unsaturated or heteroaromatic; unless defined otherwise, it preferably contains one or more, in particular 1, 2 or 3, heteroatoms in the heterocyclic ring, preferably selected from the group consisting of N, O and S; it is preferably an aliphatic heterocyclyl radical having 3 to 7 ring atoms or a heteroaromatic radical having 5 or 6 ring atoms. The heterocyclic radical can, for example, be a heteroaromatic radical or ring (heteroaryl), such as, for example, a mono-, bi- or polycyclic aromatic system, in which at least 1 ring contains one or more heteroatoms. It is preferably a heteroaromatic ring having one heteroatom selected from the group consisting of N, O and S, for example pyridyl, pyrrolyl, thienyl or furyl; furthermore, preferably, it is a corresponding heteroaromatic ring having 2 or 3 heteroatoms, for example pyrimidinyl, pyridazinyl, pyrazinyl, triazinyl, thiazolyl, thiadiazolyl, oxazolyl, isoxazolyl, pyrazolyl, imidazolyl and triazolyl. Furthermore preferably, it is a partially or fully hydrogenated heterocyclic radical having one heteroatom selected from the group consisting of N, O and S, for example oxiranyl, oxetanyl, oxolanyl (=tetrahydrofuryl), oxanyl, pyrrolidyl (=pyrrolidinyl) or piperidyl or else pyrrolinyl, such as Δ¹-pyrrolinyl, Δ²-pyrrolinyl or Δ³-pyrrolinyl, for example Δ¹-pyrrolin-2-yl, Δ¹-pyrrolin-3-yl, Δ¹-pyrrolin-4-yl or Δ¹-pyrrolin-5-yl or Δ²-pyrrolin-1-yl, Δ²-pyrrolin-2-yl, Δ²-pyrrolin-3-yl, Δ²-pyrrolin-4-yl, Δ²-pyrrolin-5-yl or Δ³-pyrrolin-1-yl, Δ³-pyrrolin-2-yl or Δ³-pyrrolin-3-yl. Furthermore, preferably, it is a partially or fully hydrogenated heterocyclic radical having 2 heteroatoms selected from the group consisting of N, O and S, for example piperazinyl, dioxolanyl, oxazolinyl, isoxazolinyl, oxazolidinyl, isoxazolidinyl and morpholinyl.

Possible substituents for a substituted heterocyclic radical are the substituents mentioned further below, and additionally also oxo. The oxo group can also be present on the ring heteroatoms which can exist in different oxidation states, for example at N and S.

Substituted radicals, such as a substituted alkyl, alkenyl, alkynyl, aryl, phenyl, benzyl, heterocyclyl and, specifically, heteroaryl radical, are, for example, a substituted radical derived from the unsubstituted skeleton, where the substituents are, for example, one or more, preferably 1, 2 or 3, radicals selected from the group consisting of halogen, alkoxy, haloalkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino, such as acylamino, mono- and dialkylamino, and alkylsulfinyl, haloalkylsulfinyl; alkylsulfonyl, haloalkylsulfonyl and, in the case of cyclic radicals, also alkyl and haloalkyl; the “substituted radicals”, such as substituted alkyl and the like, include as substituents, in addition to the saturated hydrocarbon-containing radicals mentioned, corresponding unsaturated aliphatic and aromatic radicals, such as unsubstituted or-substituted alkenyl, alkynyl, alkenyloxy, alkynyloxy, phenyl, phenoxy etc.-Substituted cyclic radicals having aliphatic moieties in the ring also include cyclic systems having substituents which are attached to the ring via a double bond, for example those substituted by an alkylidene group, such as methylidene or ethylidene.

The definition “substituted by one or more radicals” refers, unless otherwise defined, to one or more identical or different radicals.

The substituents mentioned by way of example (“first substituent level”) can, if they contain hydrocarbon-containing moieties, be, if appropriate, substituted further in these moieties (“second substituent level”), for example by one of the substituents defined for the first substituent level. Corresponding further substituent levels are possible. The term “substituted radical” preferably embraces only one or two substituent levels.

Preferred substituents for the substituent levels are, for example, amino, hydroxyl, halogen, nitro, cyano, mercapto, carboxyl, carbamoyl, SF₅, aminosulfonyl, alkyl, cycloalkyl, alkenyl, cycloalkenyl, alkynyl, monoalkyl-amino, dialkylamino, n-alkanoylamino, alkoxy-alkenyloxy, alkynyloxy, cycloalkoxy, cycloalkenyloxy, alkoxycarbonyl, alkenyloxycarbonyl, alkynyloxycarbonyl, aryloxycarbonyl, alkanoyl, alkenylcarbonyl, alkynylcarbonyl, arylcarbonyl, alkylthio, cycloalkylthio, alkenylthio, cycloalkenylthio, alkynylthio, alkylsulfinyl, alkylsulfonyl, monoalkylaminosulfonyl, dialkyl-aminosulfonyl, n-alkyl-aminocarbonyl, N,N-dialkyl-aminocarbonyl, N-alkanoyl-amino-carbonyl, N-alkanoyl-N-alkyl-aminocarbonyl, aryl, aryloxy, benzyl, benzyloxy, benzylthio, arylthio, arylamino and benzylamino.

Among the radicals with carbon atoms, preference is given to those having 1 to 6 carbon atoms, preferably 1 to 4 carbon atoms, in particular 1 or 2 carbon atoms. In general, preference is given to substituents selected from the group consisting of halogen, for example fluorine and chlorine, (C₁-C₄)alkyl, preferably methyl or ethyl, (C₁-C₄)haloalkyl, preferably trifluoromethyl, (C₁-C₄)alkoxy, preferably methoxy or ethoxy, (C₁-C₄)haloalkoxy, nitro and cyano. Particular preference is given here to the substituents methyl, methoxy and chlorine. Mono- or disubstituted amino is a chemically stable radical from the group of the substituted amino radicals which are N-substituted, for example, by one or two identical or different radicals selected from the group consisting of alkyl, alkoxy, acyl and aryl; preferably monoalkylamino, dialkylamino, acylamino, arylamino, N-alkyl-N-arylamino and N-heterocycles; preference is given to alkyl radicals having 1 to 4 carbon atoms; aryl is preferably phenyl or substituted phenyl; for acyl, the definition mentioned further below applies, preferably (C₁-C₄)alkanoyl. This applies correspondingly to substituted hydroxylamino or hydrazino. Unsubstituted or substituted phenyl is preferably phenyl which is unsubstituted or mono- or polysubstituted, preferably up to trisubstituted, by identical or different radicals selected from the group consisting of halogen, (C₁-C₄)alkyl, (C₁-C₄)alkoxy, (C₁-C₄)haloalkyl, (C₁-C₄)haloalkoxy and nitro, for example o-, m- and p-tolyl, dimethylphenyls, 2-, 3- and 4-chlorophenyl, 2-, 3- and 4-trifluoro- and -trichloromethylphenyl, 2,4-, 3,5-, 2,5- and 2,3-dichlorophenyl, o-, m- and p-methoxyphenyl.

An acid radical of an inorganic or organic oxygen acid is a radical which is formally formed by removing a hydroxyl group from the acid function, for example the sulfo radical —SO₃H, which is derived from sulfuric acid H₂SO₄, or the sulfino radical —SO₂H, which is derived from sulfurous acid H₂SO₃, or, correspondingly, the group SO₂NH₂, the phospho radical —PO(OH)₂, the group —PO(NH₂)₂, —PO(OH)(NH₂), —PS(OH)₂, —PS(NH₂)₂ or —PS(OH)(NH₂), the carboxyl radical COOH, which is derived from carbonic acid, radicals of the formula —CO—SH, —CS—OH, —CS—SH, —CO—NH₂, —CS—NH₂, —C(═NH)—OH or —C(═NH)—NH₂; also possible are radicals with hydrocarbon radicals or substituted hydrocarbon radicals, i.e., acyl radicals in the wider sense (=“acyl”).

Acyl is a radical of an organic acid which is formally formed by removing a hydroxyl group from the acid function, where the organic radical in the acid can also be attached to the acid function via a heteroatom. Examples of acyl are the radical —CO—R of a carboxylic acid HO—CO—R and radicals of acids derived therefrom, such as thiocarbonic acid, unsubstituted or N-substituted iminocarboxylic acids or the radical of carbonic monoesters, N-substituted carbamic acid, sulfonic acids, sulfinic acids, N-substituted sulfonamide acids, phosphonic acids, phosphinic acids.

Acyl is, for example, formyl, alkylcarbonyl, such as (C₁-C₄)alkylcarbonyl, phenylcarbonyl, alkyloxycarbonyl, phenyloxycarbonyl, benzyloxycarbonyl, alkylsulfonyl, alkylsulfinyl, N-alkyl-1-iminoalkyl and other radicals of organic acids. The radicals can in each case be further substituted in the alkyl or phenyl moiety, for example in the alkyl moiety by one or more radicals selected from the group consisting of halogen, alkoxy, phenyl and phenoxy; examples of substituents in the phenyl moiety are the substituents which have already been mentioned further above generally for substituted phenyl. Acyl is preferably an acyl radical in the more restricted sense, i.e. a radical of an organic acid where the acid group is directly attached to the carbon atom of an organic radical, for example formyl, alkylcarbonyl, such as acetyl or (C₁-C₄)alkylcarbonyl, phenylcarbonyl, alkylsulfonyl, alkylsulfinyl and other radicals of organic acids.

If W is “H₂”, the combined group C═W corresponds to a group of the formula CH₂.

The invention also provides all stereoisomers which are embraced by the formula (I) and mixtures thereof. Such compounds of the formula (I) contain one or more asymmetric carbon atoms (=asymmetrically substituted carbon atoms) or else double bonds, which are not specifically mentioned in the general formula (I). The possible stereoisomers, which are defined by their specific spatial form, such as enantiomers, diastereomers, Z and E isomers, are all embraced by the formula (I) and can be obtained by customary methods from mixtures of the stereoisomers or else be prepared by stereoselective reactions in combination with the use of stereochemically pure starting materials.

In particular for reasons of better herbicidal activity, better selectivity and/or better preparation, those novel compounds of the formula (I) mentioned or their salts are of particular interest in which individual radicals have one of the preferred meanings already mentioned or mentioned hereinbelow, or, in particular, those in which one or more of the preferred meanings already mentioned or mentioned hereinbelow are combined.

Particular interest attaches to compounds of the formula (I) according to the invention, and their salts, in which

A is a radical as defined under (a1) to (a5), where

(a1) is unsubstituted phenyl or unsubstituted monocyclic heteroaryl having from 3 to 7 ring atoms, preferably 5 or 6 ring atoms, and containing 1-4 ring heteroatoms, especially from 1 to 3 identical or different ring heteroatoms from the group N, O and S, with particular preference a phenyl radical, pyrazolyl, thienyl, diazathiolyl or triazinyl,

(a2) is a radical as defined under (a1) which is further substituted by one or more fused-on nonaromatic carbocyclic rings having in each case 5 or 6 ring atoms or heterocyclic rings having in each case 5 or 6 ring atoms and one or more ring heteroatoms, preferably from 1 to 4 ring heteroatoms, especially from 1 to 3 ring heteroatoms from the group N, O, and S,

(a3) is a radical as defined under (a1) or (a2) which is further substituted as defined above, preferably by one or more identical or different radicals from the group consisting of amino, hydroxyl, halogen, preferably fluorine and chlorine, nitro, cyano, mercapto, carboxyl, carbamoyl, SF₅, aminosulfonyl, (C₁-C₆)-alkyl, (C₃-C₆)-cycloalkyl, mono-((C₁-C₆)-alkyl)-amino, di-((C₁-C₆)-alkyl)-amino, N-((C₁-C₆)-alkanoyl)-amino, (C₁-C₆)-alkoxy, (C₃-C₉)-cycloalkoxy, aryloxycarbonyl, (C₁-C₆)-alkanoyl, arylcarbonyl, (C₁-C₆)-alkylthio, (C₁-C₆)-alkylsulfinyl, (C₁-C₆)-alkylsulfonyl, mono-((C₁-C₆)-alkyl)-aminosulfonyl, di-((C₁-C₆-alkyl)-aminosulfonyl, N-((C₁-C₆)-alkyl)-aminocarbonyl, N,N-di-((C₁-C₆)-alkyl)-aminocarbonyl, N-((C₁-C₆)-alkanoyl)-aminocarbonyl, N-((C₁-C₆)-alkanoyl)-N-((C₁-C₆)-alkyl)-aminocarbonyl, aryl, aryloxy, each of the above hydrocarbon substituents for (a3) being unsubstituted or substituted by one or more identical or different radicals from the group consisting of amino, hydroxyl, halogen, nitro, cyano, mercapto, carboxyl, carbamoyl, aminosulfonyl, (C₃-C₆)-cycloalkyl, mono-((C₁-C₄)-alkyl)-amino, di-((C₁-C₄)-alkyl)-amino, N-((C₁-C₆)-alkanoyl)-amino, N-((C₁-C₆)-alkanoyl)-N-((C₁-C₄)-alkyl)-amino, (C₁-C₆)-alkoxy, (C₁-C₆)-alkoxy-(C₁-C₆)-alkoxy, (C₃-C₆)-cycloalkoxy, (C₁-C₆)-haloalkoxy, ((C₁-C₄)-alkoxy)-carbonyl, (C₁-C₆)-alkanoyl, arylcarbonyl, (C₁-C₄)-alkylthio, (C₁-C₄)-alkylsulfinyl, (C₁-C₄)-alkylsulfonyl, mono-((C₁-C₄)-alkyl)-aminosulfonyl, di-((C₁-C₄)-alkyl)-aminosulfonyl, N-((C₁-C₄)-alkyl)-aminocarbonyl, N,N,-di-((C₁-C₄)-alkyl)-aminocarbonyl, N-((C₁-C₄)-alkanoyl)-aminocarbonyl and N-((C₁-C₄)-alkanoyl)-N-((C₁-C₄)-alkyl)-aminocarbonyl and, in the case of cyclic radicals, additionally (C₁-C₄)-alkyl, (C₁-C₄)-haloalkyl, (C₂-C₄)-alkoxy-(C₁-C₄)-alkyl, (C₁-C₄)-alkoxycarbonyl-(C₁-C₄)-alkyl, ((C₁-C₄)-alkylthio)-(C₁-C₄)-alkyl, mono-(C₁-C₄)-alkylamino-(C₁-C₄)-alkyl and di-(C₁-C₄)-alkyl)-amino-(C₁-C₄)-alkyl,

(a4) is (C₁-C₆)-alkyl or (C₃-C₉)-cycloalkyl,

(a5) is a hydrocarbon radical as defined under (a4) which further carries one or more identical or different radicals from the group of the radicals of subgroups (a5.1) and (a5.2), where

the subgroup (a5.1) is composed of the radicals halogen, preferably fluorine or chlorine, amino, hydroxyl, nitro, cyano, mercapto, carboxyl, carbamoyl, SF₅, aminosulfonyl, (C₃-C₉)-cycloalkyl, mono-(C₁-C₆)-alkylamino, di-((C₁-C₆)-alkyl)-amino, N-((C₁-C₆)-alkanoyl)-amino, (C₁-C₆)-alkoxy, (C₃-C₉)-cycloalkoxy, ((C₁-C₆)-alkoxy)-carbonyl, (C₁-C₆)-alkanoyl, C₁-C₆)-alkylthio, (C₁-C₆)-alkylsulfinyl, (C₁-C₆)-alkylsulfonyl, mono-((C₁-C₆)-alkyl)-aminosulfonyl, di-((C₁-C₆)-alkyl)-aminosulfonyl, N-((C₁-C₆)-alkyl)-aminocarbonyl, N,N-di-((C₁-C₆)-alkyl)-aminocarbonyl, N-((C₁-C₆)-alkanoyl)-aminocarbonyl, N-((C₁-C₆)-alkanoyl)-N-((C₁-C₆)-alkyl)-aminocarbonyl and in the case of cyclic radicals, additionally (C₁-C₄)-alkyl, (C₁-C₄)-alkoxy-(C₁-C₄)-alkyl, ((C₁-C₄)-alkoxy)-carbonyl-(C₁-C₄)-alkyl, (C₁-C₄)-alkylthio-(C₁-C₄)-alkyl, mono-((C₁-C₄)-alkyl)-amino-(C₁-C₄)-alkyl and di-((C₁-C₄)-alkyl)-amino-(C₁-C₄)-alkyl, each of the above hydrocarbon substituents (a5.1) being unsubstituted or substituted by one or more identical or different radicals from the group consisting of halogen, nitro, cyano, thiocyanato, (C₁-C₄)-alkoxy, (C₁-C₄)-haloalkoxy and (C₁-C₄)-alkylthio and, in the case of cyclic radicals, additionally (C₁-C₄)-alkyl and halo-(C₁-C₄)-alkyl, and

the subgroup (a5.2) is composed of the radicals aryl, aryloxy, arylthio, heteroaryloxy and heteroarylthio,

each of the last-mentioned 6 radicals being unsubstituted or substituted by one or more identical or different radicals from the group [=substituent group (a5.2.1)] consisting of halogen, amino, hydroxyl, nitro, cyano, mercapto, carboxyl, carbamoyl, aminosulfonyl, (C₁-C₄)-alkyl, (C₁-C₄)-haloalkyl, ((C₁-C₄)-alkoxy)-carbonyl-(C₁-C₄)-alkyl, mono-((C₁-C₄)-alkyl)-amino-(C₁-C₄)-alkyl and di-((C₁-C₄)-alkyl)-amino-(C₁-C₄)-alkyl, (C₂-C₆)-cycloalkyl, mono-((C₁-C₄)-alkyl)-amino, di-((C₁-C₄)-alkyl)-amino, N-((C₁-C₆)-alkanoyl)-amino, N-((C₁-C₆)-alkanoyl)-N-((C₁-C₄)-alkyl)-amino, (C₂-C₆)-alkenyloxy, ((C₁-C₄)-alkoxy)-carbonyl, (C₁-C₆)-alkanoyl, (C₁-C₄)-alkylthio, (C₃-C₆)-cycloalkylthio, (C₁-C₄)-alkylsulfinyl, (C₁-C₄)-alkylsulfonyl, mono-(C₁-C₄)-alkylaminosulfonyl, di-((C₁-C₄)-alkyl)-aminosulfonyl, N-((C₁-C₄)-alkyl)-aminocarbonyl, N,N-di-((C₁-C₄)-alkyl)-amino-carbonyl, N-((C₁-C₄)-alkanoyl)-aminocarbonyl and N-((C₁-C₄)-alkanoyl)-N-((C₁-C₄)-alkyl)-aminocarbonyl, each of the hydrocarbon substituents of the above substituent subgroup (a5.2.1) being unsubstituted or substituted by one or more identical or different radicals from the group consisting of halogen, nitro, cyano, thiocyanato, (C₁-C₄)-alkoxy, (C₁-C₄)-haloalkoxy and (C₁-C₄)-alkylthio and, in the case of cyclic radicals, additionally (C₁-C₄)-alkyl and halo-(C₁-C₄)-alkyl.

Particular preference is given to compounds of the formula (I) according to the invention, and salts thereof, in which A is an unsubstituted or substituted phenyl or unsubstituted or substituted monocyclic heteroaryl having 5 or 6 ring atoms and containing 1-3 identical or different ring heteroatoms from the group N and S, with particular preference a phenyl radical, pyrazolyl, thienyl, diazathiolyl or triazinyl. Where the abovementioned phenyls or monocyclic heteroaryls are substituted, they are preferably substituted by one or more identical or different radicals from the group —NH₂, F, Cl, —CH₃, —CF₃, —OCF₃, —OCHF₂ or —CF(CH₃)₂.

Preferred compounds of the formula (I) are those in which A is an unsubstituted phenyl or a substituted phenyl carrying one or more identical or different substituents, the substituents being selected from the group consisting of methyl, methoxy, ethoxy, fluorine, chlorine, bromine, iodine, trifluoromethyl, trifluoromethoxy, difluoromethyl, difluoromethoxy, trichloromethyl, trichloromethoxy, dichloromethoxy, fluoromethoxy, chloromethoxy, tetrafluoroethoxy, trifluoroethoxy, trichloroethoxy, difluoroethoxy and dichloroethoxy.

Likewise preferred compounds of the formula (I) are those in which A is a five- or six-membered aromatic ring system containing at least one or more identical or different heteroatoms from the group consisting of nitrogen, oxygen or sulfur, and the ring system is unsubstituted or carries one or more identical or different substituents, the substituents being selected from the group consisting of methyl, methoxy, ethoxy, fluorine, chlorine, bromine,- iodine, trifluoromethyl, trifluoromethoxy, difluoromethyl, difluoromethoxy, trichloromethyl, trichloromethoxy, dichloromethoxy, fluoromethoxy, chloromethoxy, tetrafluoroethoxy, trifluoroethoxy, trichloroethoxy, difluoroethoxy and dichloroethoxy.

V is preferably O or S. Additionally, V is preferably a divalent group of the formula CH₂, CHR³ or CR³R⁴, in which

R³ is selected from the group consisting of

(C₁-C₄)-alkyl, (C₂-C₄)-alkenyl, (C₂-C₄)-alkynyl, (C₃-C₆)-cycloalkyl, (C₅-C₆)-cycloalkenyl, mono-(C₁-C₆)-alkylamino, di-((C₁-C₆)-alkyl)-amino, N-(C₁-C₆)-alkanoyl)-amino, (C₁-C₄)-alkoxy, (C₂-C₄)-alkenyloxy, (C₂-C₄)-alkynyloxy, (C₃-C₆)-cycloalkoxy, (C₅-C₆)-cycloalkenyloxy, (C₁-C₄)-alkylthio, (C₃-C₆)-cycloalkylthio, (C₂-C₄)-alkenylthio, (C₅-C₆)-cycloalkenylthio, (C₂-C₄)-alkynylthio, (C₁-C₄)-alkanoyl, ((C₂-C₄)-alkenyl)-carbonyl, ((C₂-C₄)-alkynyl)-carbonyl, arylcarbonyl, ((C₁-C₄)-alkoxy)-carbonyl, ((C₂-C₄)-alkenyloxy)-carbonyl, ((C₂-C₄)-alkynoxy)-carbonyl, aryloxycarbonyl, (C₁-C₄)-alkylsulfinyl and (C₁-C₄)-alkylsulfonyl, each of the above radicals indicated for R³ being unsubstituted or substituted by halogen or cyano, R³ being in particular methyl, ethyl, n-propyl, 1-propyl or cyclopropyl, especially methyl,

and R⁴ independently of R³ being one of the radicals defined under R³.

Preferably, R¹ and R² independently of one another are each H or (C₁-C₄)-alkyl.

Q is preferably a divalent group of the formula O, S, SO, SO₂, NR⁵, CR⁶R⁷, CR⁶OR⁷, CO, CS or C═N—R′, in which R′ is H, OH, (C₁-C₄)-alkyl or (C₁-C₄)-alkoxy, and in the formulae

R⁶ and R⁷ independently of one another are hydrogen, (C₁-C₄)-alkyl, (C₃-C₆)-cycloalkyl, or cyano and

R⁵ is hydrogen, hydroxyl, formyl, NR⁸R⁹, (C₁-C₄)-alkyl, (C₂-C₄)-alkenyl, (C₂-C₄)-alkynyl, (C₃-C₆)-cycloalkyl, (C₅-C₆)-cycloalkenyl, (C₁-C₄)-alkyloxy, (C₂-C₄)-alkenyloxy or (C₂-C₄)-alkynyloxy, each of the last-mentioned hydrocarbon substituents for R⁵ being unsubstituted or substituted by one or more substituents from the group OR⁸, COR⁸, COOR⁸, OCOR⁸, CN, halogen, S(O)PR⁸ [where p=0, 1 or 2], NR⁸R⁹, NO₂, NR COR⁹, NR CONR⁹R¹⁰, CONR⁸R⁹ and heterocyclyl, and where each of the radicals R⁸, R⁹ and R¹⁰ independently of one another is hydrogen or (C₁-C₄)-alkyl, (C₁-C₄)-haloalkyl, and in particular R⁵ is H, OH, NH₂, mono- or di-(C₁-C₄)-alkylamino or (C₁-C₄)-alkoxy.

Q is with particular preference, O, S, SO₂, NH, N(CH₃), N(OH)—, N(NH₂), N(N(CH₃)₂), CH₂, CHCN, CH(OH), CHOCH₂.

Likewise preferred compounds of the formula (I) are those in which D is a pyridyl radical, a 1,3,5-triazinyl radical, a diazabenzene radical or a phenyl radical, each of the last-mentioned 4 radicals being unsubstituted or carrying one or two identical or different substituents, the substituents being selected from the group consisting of amino, hydroxyl, halogen, preferably fluorine and chlorine, nitro, cyano, mercapto, carboxyl, carbamoyl, SF₅, methylsulfanyl, methylsulfonyl, aminosulfonyl, cyclopropylamino, (C₁-C₆)-alkyl, (C₃-C₉)-cycloalkyl, (C₂-C₆)-alkenyl, (C₅-C₉)-cycloalkenyl, (C₂-C₆)-alkynyl, mono-((C₁-C₆)-alkyl)-amino, di-((C₁-C₆)-alkyl)-amino, N-((C₁-C₆)-alkanoyl)-amino, (C₁-C₆)-alkoxy, (C₂-C₆)-alkenyloxy, (C₂-C₆)-alkynyloxy, (C₃-C₉)-cycloalkoxy, (C₅-C₉)-cycloalkenyloxy, ((C₁-C₆)-alkoxy)-carbonyl, ((C₂-C₆)-alkenyloxy)-carbonyl, ((C₂-C₆)-alkynyloxy)-carbonyl, aryloxycarbonyl, (C₁-C₆)-alkanoyl, ((C₂-C₆)-alkenyl)-carbonyl, ((C₂-C₆)-alkynyl)-carbonyl, arylcarbonyl, (C₁-C₆)-alkylthio, (C₃-C₉)-cycloalkylthio, (C₂-C₆)-alkenylthio, (C₅-C₉)-cycloalkenylthio, (C₂-C₆)-alkynylthio, (C₁-C₆)-alkylsulfinyl, (C₁-C₆)-alkylsulfonyl, mono-((C₁-C₆)-alkyl)-aminosulfonyl, di-((C₁-C₆)-alkyl)-aminosulfonyl, N-((C₁-C₆)-alkyl)-aminocarbonyl, N, N-di-((C₁-C₆)-alkyl)-aminocarbonyl, N-((C₁-C₆)-alkanoyl)-aminocarbonyl, N-((C₁-C₆)-alkanoyl)-N-((C₁-C₆)-alkyl)-aminocarbonyl, aryl, aryloxy, benzyl, benzyloxy, benzylthio, arylthio, thiocyanato, tri-((C₁-C₆)-alkyl)-silyl, (C₁-C₆)-alkanoyloxy, (C₁-C₆)-alkylsulfonyloxy, ((C₁-C₆)-alkoxy)-carbonyloxy, ((C₂-C₆)-alkenyloxy)-carbonyloxy, ((C₂-C₆)-alkynyloxy)-carbonyloxy, N-((C₁-C₆)-alkyl)-aminocarbonyloxy, N,N,-di-((C₁ C₆)-alkyl)-aminocarbonyloxy, each of the above hydrocarbon substituents being unsubstituted or substituted by one or more identical or different radicals from the group consisting of amino, hydroxyl, halogen, nitro, cyano, mercapto, carboxyl, carbamoyl, aminosulfonyl, (C₃-C₆)-cycloalkyl, (C₂-C₄)-alkenyl, (C₅-C₉)-cycloalkenyl, (C₂-C₆)-alkynyl, mono-(C₁-C₄)-alkylamino, di-((C₁-C₄)-alkyl)-amino, N-((C₁-C₆)-alkanoyl)-amino, N-((C₁-C₆)-alkanoyl)-N-((C₁-C₄)-alkyl)-amino, (C₁-C₆)-alkoxy, (C₁-C₆)-alkoxy-(C₁-C₆)-alkoxy, (C₁-C₆)-alkoxy-[poly-(C₁-C₄)-alkenyloxy)]-, hydroxy-(C₁-C₆)-alkoxy, hydroxy-[poly-(C₁-C₄)-alkenyloxy)]-, (C₃-C₆)-cycloalkoxy, (C₁-C₆)-haloalkoxy, (C₂-C₆)-alkenyloxy, (C₅-C₉)-cycloalkenyloxy, (C₂-C₆)-haloalkenyloxy, (C₂-C₆)-alkynyloxy, (C₂-C₆)-haloalkynyloxy, ((C₁-C₄)-alkoxy)-carbonyl, aryloxycarbonyl, (C₁-C₆)-alkanoyl, arylcarbonyl, (C₁-C₄)-alkylthio, (C₃-C₆)-cycloalylkthio, (C₂-C₄) alkenylthio, (C₅-C₉)-cycloalkenylthio, (C₂-C₄)-alkynylthio, (C₁-C₄)-alkylsulfinyl, (C₁-C₄)-alkylsulfonyl, mono-((C₁-C₄)-alkyl)-aminosulfonyl, di-((C₁-C₄)-alkyl)-aminosulfonyl, N-((C₁-C₄)-alkyl)-aminocarbonyl, N,N,-di-((C₁-C₄)-alkyl)-aminocarbonyl, N-((C₁-C₄)-alkanoyl)-aminocarbonyl and N-((C₁-C₄)-alkanoyl)-N-((C₁-C₄)-alkyl)-aminocarbonyl and, in the case of cyclic radicals, additionally (C₁-C₄)-alkyl, (C₁-C₄)-haloalkyl, (C₂-C₄)-alkenyl, (C₂-C₄)-alkynyl, (C₂-C₆)-haloalkenyl, (C₂-C₆)-haloalkynyl, (C₂-C₄)-alkoxy)-(C₁-C₄)-alkyl, ((C₁-C₄)-alkoxy)carbonyl-(C₁-C₄)-alkyl, (C₁-C₄)-alkylthio-(C₁-C₄)-alkyl, mono-((C₁-C₄)-alkyl)-amino-(C₁-C₄)-alkyl and di-((C₁-C₄)-alkyl)-amino-(C₁-C₄)-alkyl. A diazabenzene radical is preferably a pyrimidin-2-yl, pyrimidin-4-yl, a pyrimidin-5-yl, an S-triazinyl, a pyridazin-3-yl, a pyridazin-4-yl, a pyridin-2-yl, a pyridin-3-yl or a pyridin-4-yl. These parent radicals are preferably unsubstituted or carry one or more identical or different substituents, the substituents being selected from the group consisting of amino, halogen, preferably fluorine and chlorine, nitro, cyano, carbamoyl, SF₅, methylsulfonyl, cyclopropylamino, (C₁-C₆)-alkyl, (C₃-C₉)-cycloalkyl, mono-((C₁-C₆)-alkyl)-amino, di-((C₁-C₆)-alkyl)-amino, (C₁-C₆)-alkoxy, (C₃-C₉)-cycloalkoxy, (C₁-C₆)-alkoxycarbonyl, aryloxycarbonyl, (C₁-C₆)-alkanoyl, (C₂-C₆)-alkenylcarbonyl, (C₂-C₆)-alkynylcarbonyl, arylcarbonyl, (C₅-C₉)-cycloalkenylthio, (C₂-C₆)-alkynylthio, (C₁-C₆)-alkylsulfinyl, (C₁-C₆)-alkylsulfonyl, mono-((C₁-C₆)-alkyl)-aminosulfonyl, d 1-((C₁-C₆)-alkyl)-aminosulfonyl, N-((C₁-C₆)-alkyl)-aminocarbonyl, N,N-di-((C₁-C₆)-alkyl)-aminocarbonyl, N-((C₁-C₆)-alkanoyl)-aminocarbonyl, N-((C₁-C₆)-alkanoyl)-N-((C₁-C₆)-alkyl)-aminocarbonyl, thiocyanato, each of the above hydrocarbon substituents being unsubstituted or substituted by one or more identical or different substituents in the group consisting of amino hydroxyl, halogen, nitro, cyano, mercapto, carboxyl, carbamoyl, aminosulfonyl, (C₃-C₆)-cycloalkyl, mono-(C₁-C₄)-alkylamino, di-((C₁-C₄)-alkyl)-amino, N-((C₁-C₆)-alkanoyl)-amino, N-((C₁-C₆)-alkanoyl)-N-((C₁-C₄)-alkyl)-amino, (C₁-C₆)-alkoxy, (C₁-C₆)-haloalkoxy, ((C₁-C₄)-alkoxy)-carbonyl, (C₁-C₆)-alkanoyl, (C₁-C₄)-alkylthio, (C₃-C₆)-cycloalkylthio, (C₁-C₄)-alkylsulfinyl, (C₁-C₄)-alkylsulfonyl, mono-((C₁-C₄)-alkyl)-aminosulfonyl, di-((C₁-C₄)-alkyl)-aminosulfonyl, N-((C₁-C₄)-alkyl)-aminocarbonyl, N,N,-di-((C₁-C₄) alkyl)-amino-carbonyl, N-((C₁-C₄)-alkanoyl)-aminocarbonyl und N-((C₁-C₄)-alkanoyl)-N-((C₁-C₄)-alkyl)-aminocarbonyl and, in the case of cyclic radicals, additionally (C₁-C₄)-alkyl, (C₁-C₄)-haloalkyl, ((C₂-C₄)-alkoxy)-(C₁-C₄)-alkyl, (C₁-C₄)-alkoxycarbonyl-(C₁-C₄)-alkyl, (C₁-C₄)-alkylthio-(C₁-C₄)-alkyl, mono-((C₁-C₄)-alkyl)-amino-(C₁-C₄)-alkyl and di-((C₁-C₄)-alkyl)-amino-(C₁-C₄)-alkyl.

Further preferred compounds of the formula (I) are those in which D is unsubstituted or carries one or more identical or different substituents, the substituents being selected from the group consisting of amino, halogen, preferably fluorine and chlorine, cyano, methylsulfonyl, cyclopropylamino, (C₁-C₆)-alkyl, (C₃-C₆)-cycloalkyl, mono-((C₁-C₆)-alkyl)-amino, di-((C₁-C₆)-alkyl)-amino, N-((C₁-C₆)-alkanoyl)-amino, (C₁-C₆)-alkoxy, (C₁-C₆)-alkylthio, (C₃-C₉)-cycloalkylthio, (C₂-C₆)-alkenylthio, (C₅-C₉)-cycloalkenylthio, (C₂-C₆)-alkynylthio, (C₁-C₆)-alkylsulfinyl, (C₁-C₆)-alkylsulfonyl, each of the above hydrocarbon substituents being unsubstituted or substituted by one or more identical or different radicals from the group consisting of amino, halogen, cyano, mono-(C₁-C₄)-alkylamino, di-((C₁-C₄)-alkyl)-amino, N-((C₁-C₆)-alkanoyl)-amino, N-((C₁-C₆)-alkanoyl)-N-((C₁-C₄)-alkyl)-amino, (C₁-C₆)-alkoxy, (C₁-C₆)-haloalkoxy, (C₁-C₄)-alkylthio, (C₁-C₄)-alkylsulfonyl and, in the case of cyclic radicals, additionally (C₁-C₄)-alkyl, (C₁-C₄)-haloalkyl, mono-((C₁-C₄)-alkyl)-amino-(C₁-C₄)-alkyl and di-((C₁-C₄)-alkyl)-amino-(C₁-C₄)-alkyl.

Compounds of the formula (I) may be obtained by means of various synthesis steps, of which some are given by way of example below. The compounds of the formulae (II) to (VII) are known or may be prepared in analogy to known processes.

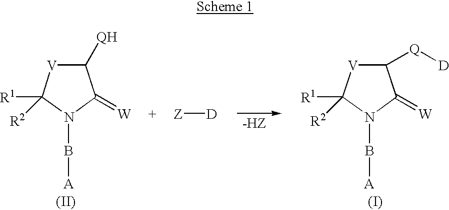

Compounds of the formula (I) in which Q=O, S or NR⁵ or salts thereof may be prepared (cf. scheme 1) by reacting a compound of formula Z-D, in which Z is an exchangeable radical or a leaving group, e.g. fluorine, chlorine, bromine, iodine, trichloromethyl, (C₁-C₄)-alkylsulfonyl, unsubstituted or substituted phenyl-(C₁-C₄)-alkylsulfonyl or (C₁-C₄)-alkyl-phenylsulfonyl, with an appropriate compound of the formula (II) (see scheme) or an acid addition salt thereof, the radicals R¹, R², A, B, V, W, and D in the formulae (II) and Z-D being as defined in formula (I).

The reaction of the compounds of the formula (II) and Z-D takes place preferably with base catalysis in an inert organic solvent, such as THF, dioxane, acetonitrile, DMF (dimethylformamide), methanol and ethanol, for example, or in a solvent mixture at temperatures between −10° C. and the boiling point of the respective solvent or solvent mixture, preferably at from 20° C. to 60° C., with the compound (II), if used as an acid addition salt, being liberated in situ with a base, where appropriate. Suitable bases and basic catalysts include alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1,8-diazabicyclo[5.4.0]undec-7-ene (DBU). The respective base is generally used in the range from 1 to 3 molar equivalents relative to the compound of the formula Z-D. The compound of the formula Z-D may be used, for example, in an equimolar amount to the compound of the formula (II) or in excess, generally with an excess of up to 2 molar equivalents. The corresponding processes are basically known from the literature (cf. Comprehensive Heterocyclic Chemistry, A. R. Katritzky, C. W. Rees, Pergamon Press, Oxford, N.Y., 1984, Vol.3; Part 2B, p. 482).

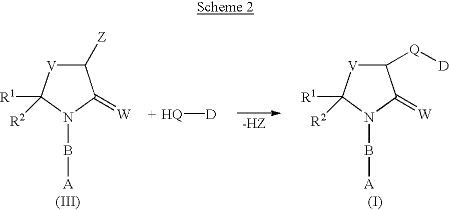

Another possible way of preparing compounds of the formula (I) in which Q=O, S or NR⁵ or salts thereof (cf. scheme 2) comprises reacting a compound of the formula (III) (see scheme 2) in which Z is an exchangeable radical or a leaving group, e.g., chlorine, bromine, iodine, trichloromethyl, (C₁-C₄)-alkylsulfonyl, unsubstituted or substituted phenyl-(C₁-C₄)-alkylsulfonyl or (C₁-C₄)-alkyl-phenylsulfonyl with an appropriate compound of the formula HQ-D or an acid addition salt thereof, where the radicals R¹, R², R⁵, A, B, V, W and D in the formulae (III) in HQ-D are as defined in formula (I).

The reaction of the compounds of the formula (III) and HQ-D takes place preferably with base catalysis in an inert organic solvent, such as THF, dioxane, acetonitrile, DMF, methanol and ethanol, for example, at temperatures between −10° C. and the boiling point of the respective solvent or solvent mixture, preferably at from 20° C. to 60° C., with the compound HQ-D, if used as an acid addition salt, being liberated in situ with a base, where appropriate. Suitable bases and basic catalysts include alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1,8-diazabicyclo[5.4.0]undec-7-ene (DBU). The respective base is generally used in the range from 1 to 3 molar equivalents relative to the compound of the formula HQ-D. The compound of the formula HQ-D may be used, for example, in an equimolar amount to the compound of the formula (III) or in a substoichiometric or superstoichiometric (excess) amount. The corresponding processes are basically known from the literature (cf. Comprehensive Heterocyclic Chemistry, A. R. Katritzky, C. W. Rees, Pergamon Press, Oxford, N.Y., 1984, Vol.3; Part 2B, p. 482).

Further compounds of the formula (I) in which Q is a radical of the formula —N—(NH₂)— can be obtained by aminating the bridge nitrogen in the compound (I) where Q=NH [e.g., Stroh, R. (ed.): Houben-Weyl “Methoden der organischen Chemie” volume 10/2 p. 36 ff. (1967)].

Further compounds of the formula (I) where Q=NOH, SO, SO₂ can be obtained by oxidizing the bridge heteroatom in the compounds (I) where Q=NH or S [e.g. Stroh, R. (ed.): Houben-Weyl “Methoden der organischen Chemie” volume 10/1 p. 1135 ff. (1971) or Müller, E. (ed.): Houben-Weyl “Methoden der organischen Chemie” volume 9 p. 207 ff. (1955)].

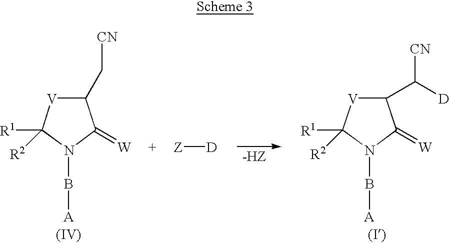

Compounds of the formula (I) in which Q is a radical of the formula CHCN or salts thereof (compound (I′)) may be prepared (cf. scheme 3) by reacting a compound of the formula Z-D, in which Z is an exchangeable radical or leaving group, e.g., fluorine, chlorine, bromine, iodine, trichloromethyl, (C₁-C₄)-alkylsulfonyl, unsubstituted or substituted phenyl-(C₁-C₄)-alkylsulfonyl or (C₁-C₄)-alkyl-phenylsulfonyl with an appropriate compound of the formula (IV) (see scheme 3), the radicals R¹, R², A, B, V, W and D in the formulae (IV) and Z-D being as defined for formula (I).

The reaction of the compounds of the formula (IV) and Z-D takes place preferably with base catalysis in an inert organic solvent, such as THF, dioxane, acetonitrile, DMF, methanol and ethanol, for example, at temperatures between −10° C. and the boiling point of the respective solvent or solvent mixture, preferably at from 20° C. to 60° C. Suitable bases and basic catalysts include alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1,8-diazabicyclo[5.4.0]undec-7-ene (DBU). The respective base is generally used in the range from 1 to 3 molar equivalents relative to the compound of the formula Z-D. The compound of the formula Z-D may be used, for example, in an equimolar amount to the compound of the formula (IV) or in a substoichiometric or superstoichiometric (excess) amount. The corresponding processes and also the preparation of the precursors are basically known from the literature [Kornet, M. J.; J. Heterocycl. Chem. 22(1) 129-130 (1985) and Haider, N.; Heinisch, G.; Moshuber, J.; Heterocycles 38(1) 125-134 (1994)].

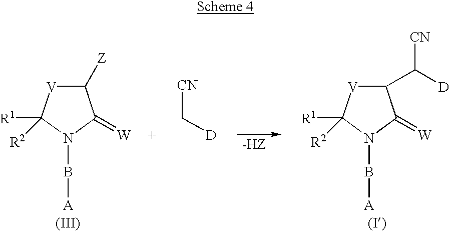

A further possible way of preparing compounds of the formula (I) in which Q=CHCN or salts thereof (formula (1′)) comprises (cf. scheme 4) reacting a compound of the formula (III) (see scheme 4) in which Z is an exchangeable radical or a leaving group, e.g., chlorine, bromine, iodine, trifluoromethyl, (C₁-C₄)-alkylsulfonyl, unsubstituted or substituted phenyl-(C₁-C₄)-alkylsulfonyl or (C₁-C₄)-alkyl-phenylsulfonyl with an appropriate compound of the formula NC—CH₂-D, the radicals R¹, R², A, B, V, W and D in the formulae (III) and NC—CH₂-D being as defined for formula (I). The reaction of the compounds of the formula (III) and NC—CH₂-D takes place preferably with base catalysis in an inert organic solvent, such as THF, dioxane, acetonitrile, DMF, methanol and ethanol, for example, at temperatures between −10° C. and the boiling point of the respective solvent or solvent mixture, preferably at from 20° C. to 60° C. Suitable bases and basic catalysts include alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1,8-diazabicyclo[5.4.0]undec-7-ene (DBU). The respective base is generally used in the range from 1 to 3 molar equivalents relative to the compound of the formula NC—CH₂-D. The compound of the formula NC—CH₂-D may be used, for example, in an equimolar amount to the compound of the formula (III) or in a substoichiometric or superstoichiometric (excess) amount. The corresponding processes are basically known from the literature [Haider, N.; Heinisch, G.; Moshuber, J.; Heterocycles 38(1) 125-134 (1994)]. Processes for preparing compounds of the formula NC—CH₂-D are likewise known from the literature [Furakawa, M. Chem. Pharm. Bull. 10 1215 (1962)].

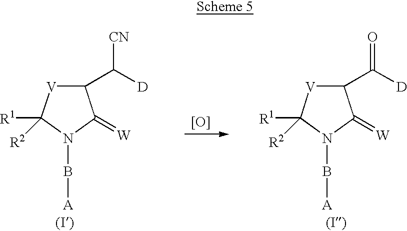

The conversion of compounds of the type (I′) (see scheme 5) into carbonyl compounds of the type (I″) is likewise known from the literature [Haider, N.; Heinisch, G.; Moshuber, J.; Heterocycles 38(1) 125-134 (1994)]: From the carbonyl compounds of the type (I″) further compounds of the formula (I′) where Q=(C—OH), CH₂, (C═NOH), (C═NOMe), etc are obtainable by standard literature processes.

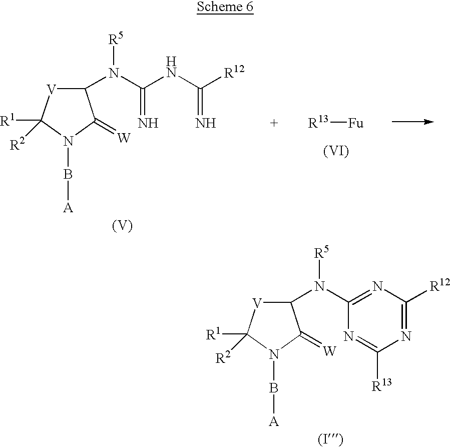

where D is a radical of the formula (IX)

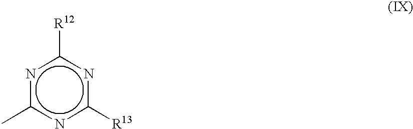

and Q=NR⁵, compounds of the formula (I) (compound (I′″)) or salts thereof may also be synthesized (cf. scheme 6) by reacting appropriate compounds of the formula (V) (see scheme 6) or an acid addition salt thereof with compounds of the formula (VI)

R¹³-Fu (VI)

in which Fu is a functional group from the group consisting of carboxylate, orthocarboxylate, carbonyl chloride, carboxamide, carboxylic anhydride, and trichloromethyl.

The reaction of the compounds of the formula (V) and (VI) takes place preferably with base catalysis in an inert organic solvent, such as tetrahydrofuran (THF), dioxane, acetonitrile, dimethylformamide (DMF), methanol and ethanol, for example, at temperatures between −10° C. and the boiling point of the solvent, preferably at from 20° C. to 60° C.; where acid addition salts of the formula (V) are used, they are generally liberated in situ by means of a base. Suitable bases and basic catalysts include alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1,8-diazabicyclo[5.4.0]undec-7-ene (DBU). The respective base is used, for example, in the range from 0.1 to 3 molar equivalents relative to the compound of the formula (V). The compound of the formula (VI) may be used in proportion to the compound of the formula (V), for example, in an equimolar amount or in excess, generally in a molar ratio (V):(VI) of up to 1:4, generally up to 1:3. The corresponding processes are basically known in the literature (cf.

Comprehensive Heterocyclic Chemistry, A. R. Katritzky, C. W. Rees, Pergamon Press, Oxford, N.Y., 1984, Vol.3; Part 2B, p. 290). The radicals R¹, R², R⁵, A, B, V and W are as defined in formula (I).

R¹² and R¹³ correspond to the substituents in the radical D of the formula (I) in the case of a substituted 1,3,5-triazine radical and are preferably defined, in each case independently of one another, as a radical from the group consisting of amino, hydroxyl, halogen, preferably fluorine and chlorine, nitro, cyano, mercapto, carboxyl, carbamoyl, methylsulfanyl, methylsulfonyl, aminosulfonyl, and cyclopropylamino.

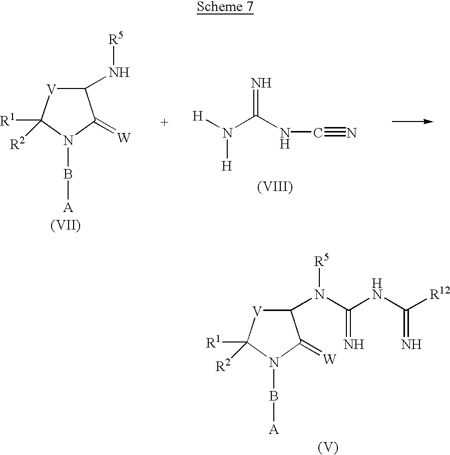

The compounds of the formula (V) may be prepared from compounds of the formula (VII) and/or their acid adducts (cf. scheme 7) by reaction with cyanoguanides (“dicyandiamide”) of the formula (VIII), where appropriate in the presence of a reaction auxiliary, such as hydrochloride, for example, and where appropriate in the presence of a diluent, such as n-decane or 1,2-dichlorobenzene, for example, at temperatures of, for example, between 100° C. and 200° C. (cf. EP-A-492615, Preparation Examples).

The pyridazines/pyrimidines of the invention may be synthesized in accordance with the general specifications as described in Houben-Weyl, Methods of Organic Chemistry, Hetarenes IV volume E 9b/part 1; 1-372 (1998) (E. Schaumann, ed.).

The pyridines of the invention may be synthesized in accordance with the general specifications as described in Houben-Weyl, Methods of Organic Chemistry, Hetarenes II-part 2 volume E 7b, 286-686 (1992) (Richard Kreher, ed.).

The triazines of the invention may be synthesized in accordance with the general specifications as described in patent application WO 00/02868 (processes for preparing 2-amino-4-chloro-1,3,5-triazines).

Also possible for preparing enantiomers of the compounds (I) are customary methods for optical resolution (cf. textbooks of stereochemistry), for example following processes for separating mixtures into diastereomers, for example physical processes, such as crystallization, chromatographic processes, in particular column chromatography and high pressure liquid chromatography, distillation, if appropriate under reduced pressure, extraction and other processes, it is possible to separate the remaining mixtures of enantiomers, generally by chromatographic separation on chiral solid phases. Suitable for preparative amounts or on an industrial scale are processes such as the crystallization of diastereomeric salts which can be obtained from the compounds (I) using optically active acids and, if appropriate, provided that acidic groups are present, using optically active bases.

Optically active acids which are suitable for optical resolution by crystallization of diastereomeric salts are, for example, camphorsulfonic acid, camphoric acid, bromocamphorsulfonic acid, quinic acid, tartaric acid, dibenzoyltartaric acid and other analogous acids; suitable optically active bases are, for example, quinine, chinchonine, quinidine, brucine, 1-phenylethylamine and other analogous bases. The crystallizations are then in most cases carried out in aqueous or aqueous-organic solvents, where the diastereomer which is less soluble precipitates first, if appropriate after seeding. One enantiomer of the compound of the formula (I) is then liberated from the precipitated salt, or the other is liberated from the crystals, by acidification or using a base.

The following acids are suitable for preparing the acid addition salts of the compounds of the formula (I): hydrohalic acids, such as hydrochloric acid or hydrobromic acid, furthermore phosphoric acid, nitric acid, sulfuric acid, mono- or bifunctional carboxylic acids and hydroxycarboxylic acids, such as acetic acid, maleic acid, succinic acid, fumaric acid, tartaric acid, citric acid, salicylic acid, sorbic acid or lactic acid, and also sulfonic acids, such as p-toluenesulfonic acid and 1,5-naphthalenedisulfonic acid. The acid addition compounds of the formula (I) can be obtained in a simple manner by the customary methods for forming salts, for example by dissolving a compound of the formula (I) in a suitable organic solvent, such as, for example, methanol, acetone, methylene chloride or benzine, and adding the acid at temperatures from 0 to 100° C., and they can be isolated in the known manner, for example by filtration, and, if appropriate, purified by washing with an inert organic solvent.

The base addition salts of the compounds of the formula (I) are preferably prepared in inert polar solvents, such as, for example, water, methanol or acetone, at temperatures from 0 to 100° C. Examples of bases which are suitable for the preparation of the salts according to the invention are alkali metal carbonates, such as potassium carbonate, alkali metal hydroxides and alkaline earth metal hydroxides, for example NaOH or KOH, alkali metal hydrides and alkaline earth metal hydrides, for example NaH, alkali metal alkoxides and alkaline earth metal alkoxides, for example sodium methoxide or potassium tert-butoxide, or ammonia, ethanolamine or a quarternary ammonium hydroxide of the formula [NRR′R″R′″]⁺OH⁻.

Solvents referred to as “inert solvents” in the above process variants are to be understood as meaning in each case solvents which are inert under the reaction conditions in question, but which need not be inert under any reaction conditions.

A collection of compounds of the formula (I) which can be synthesized by the abovementioned process may also be prepared in a parallel manner where the process may be carried out manually, partially automated or fully automated. In this case, it is possible, for example, to automate the procedure of the reaction, the work-up or the purification of the products or of the intermediates. In total, this is to be understood as meaning a procedure as is described, for example, by S. H. DeWitt in “Annual Reports in Combinatorial Chemistry and Molecular Diversity: Automated Synthesis”, Volume 1, Verlag Escom, 1997, pages 69 to 77.

A number of commercially available apparatuses as are offered by, for example, Stem Corporation, Woodrolfe Road, Tollesbury, Essex, CM9 8SE, England or H+P Labortechnik GmbH, Bruckmannring 28, 85764 Oberschleigheim, Germany may be used for the parallel implementation of the reaction and work-up. For the parallel purification of compounds (I), or of intermediates obtained during the preparation, use may be made, inter alia, of chromatography apparatuses, for example those from ISCO, Inc., 4700 Superior Street, Lincoln, Nebr. 68504, USA. The apparatuses mentioned make possible a modular procedure in which the individual process steps are automated, but manual operations have to be performed between the process steps. This can be avoided by employing semi-integrated or fully integrated automation systems where the automation modules in question are operated by, for example, robots. Such automation systems can be obtained, for example, from Zymark Corporation, Zymark Center, Hopkinton, Mass. 01748, USA.

In addition to the methods described here, compounds of the formula (I) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described extensively in the specialist literature, for example Barry A. Bunin in “The Combinatorial Index”, Verlag Academic Press, 1998.

The use of solid-phase-supported synthesis methods permits a series of protocols which are known from the literature and which, in turn, can be performed manually or in an automated manner. For example, the “tea-bag method” (Houghten, U.S. Pat. No. 4,631,211; Houghten et al., Proc. Natl. Acad. Sci, 1985, 82, 5131-5135), in which products from IRORI, 11149 North Torrey Pines Road, La Jolla, Calif. 92037, USA, are employed, may be partially automated. The automation of solid-phase-supported parallel synthesis is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

The preparation methods described here give compounds of the formula (I) in the form of collections of substances known as libraries. The present invention therefore also provides libraries of the compounds of the formula (I) which contain at least two compounds of the formula (I) and their intermediates.

The compounds of the formula (I) according to the invention and their salts, hereinbelow together referred to as compounds of the formula (I) (according to the invention), have excellent herbicidal activity against a broad spectrum of economically important monocotyledonous and dicotyledonous harmful plants. The active compounds also act efficiently on perennial weeds which produce shoots from rhizomes, root stocks or other perennial organs and which are difficult to control. In this context, it is generally immaterial whether the substances are applied pre-sowing, pre-emergence or post-emergence. Specifically, examples may be mentioned of some representatives of the monocotyledonous and dicotyledonous weed flora which can be controlled by the compounds according to the invention, without these being intended as a restriction to certain species.

Examples of weed species on which the active compounds act efficiently are, from amongst the monocotyledons, Agrostis, Alopecurus, Apera, Avena, Brachicaria, Bromus, Dactyloctenium, Digitaria, Echinochloa, Eleocharis, Eleusine, Festuca, Fimbristylis, lschaemum, Lolium, Monochoria, Panicum, Paspalum, Phalaris, Phleum, Poa, Sagittaria, Scirpus, Setaria, Sphenoclea, and also Cyperus species from the annual sector and from amongst the perennial species such as Agropyron, Cynodon, Imperata and Sorghum, and also perennial Cyperus species.

In the case of the dicotyledonous weed species, the spectrum of action extends to species such as, for example, Galium, Viola, Veronica, Lamium, Stellaria, Amaranthus, Sinapis, Ipomoea, Matricaria, Abutilon and Sida from amongst the annuals, and Convolvulus, Cirsium, Rumex and Artemisia in the case of the perennial weeds. Moreover, herbicidal activity is observed in connection with dicotyledonous weeds such as Ambrosia, Anthemis, Carduus, Centaurea, Chenopodium, Cirsium, Convolvulus, Datura, Emex, Galeopsis, Galinsoga, Lepidium, Lindernia, Papaver, Portlaca, Polygonum, Ranunculus, Rorippa, Rotala, Seneceio, Sesbania, Solanum, Sonchus, Taraxacum, Trifolium, Urtica and Xanthium.

If the compounds according to the invention are applied to the soil surface prior to germination, then the weed seedlings are either prevented completely from emerging, or the weeds grow until they have reached the cotyledon stage but then their growth stops, and, eventually, after three to four weeks have elapsed, they die completely.

If the active compounds are applied post-emergence to the green parts of the plants, growth also stops drastically a very short time after the treatment and the weed plants remain at the developmental stage of the point in time of application, or they die completely after a certain time, so that in this manner competition by the weeds, which is harmful to the crop plants, is eliminated at a very early point in time and in a sustained manner.

Although the compounds according to the invention have an excellent herbicidal activity against monocotyledonous and dicotyledonous weeds, crop plants of economically important crops, for example wheat, barley, rye, rice, corn, sugar beet, cotton and soya, are not damaged at all, or only to a negligible extent. For these reasons, the present compounds are highly suitable for selectively controlling undesired plant growth in plantings of agriculturally useful plants.

In addition, the substances according to the invention have outstanding growth regulatory properties in crop plants. They engage in the plant metabolism in a regulating manner and can thus be employed for the targeted control of plant constituents and for facilitating harvesting, such as, for example, by provoking desiccation and stunted growth. Furthermore, they are also suitable for generally regulating and inhibiting undesirable vegetative growth, without destroying the plants in the process. Inhibition of vegetative growth plays an important role in many monocotyledonous and dicotyledonous crops because lodging can be reduced hereby or prevented completely.

Owing to their herbicidal and plant-growth-regulatory properties, the active compounds can also be employed for controlling harmful plants in crops of known or still to be developed genetically engineered plants. The transgenic plants generally have particularly advantageous properties, for example resistance to certain pesticides, in particular certain herbicides, resistance to plant diseases or causative organisms of plant diseases, such as certain insects or microorganisms such as fungi, bacteria or viruses. Other particular properties relate, for example, to the quantity, quality, storage-stability, composition and to specific ingredients of the harvested product. Thus, transgenic plants having an increased starch content or a modified quality of the starch or those having a different fatty acid composition of the harvested product are known.

The use of the compounds of the formula (I) according to the invention or their salts in economically important transgenic crops of useful and ornamental plants, for example of cereal, such as wheat, barley, rye, oats, millet, rice, maniok and corn, or else in crops of sugar beet, cotton, soya, oilseed rape, potato, tomato, pea and other vegetable species, is preferred. The compounds of the formula (I) can preferably be used as herbicides in crops of useful plants which are resistant or which have been made resistant by genetic engineering toward the phytotoxic effects of the herbicides.

Conventional ways of preparing novel plants which have modified properties compared to known plants comprise, for example, traditional breeding methods and the generation of mutants. Alternatively, novel plants having modified properties can be generated with the aid of genetic engineering methods (see, for example, EP-A 0221044, EP-A 0131624). For example, there have been described several cases of

genetically engineered changes in crop plants in order to modify the starch synthesized in the plants (for example WO 92/11376, WO 92/14827 and WO 91/19806),

transgenic crop plants which are resistant to certain herbicides of the glufosinate- (cf., for example, EP-A 0242236, EP-A 0242246) or glyphosate-type (WO 92/00377), or of the sulfonylurea-type (EP-A 0257 993, U.S. Pat. No. 5,013,659),

transgenic crop plants, for example cotton, having the ability to produce Bacillus thuringiensis toxins (Bt toxins) which give the plants resistance to certain pests (EP-A 0142924, EP-A 0193259),

transgenic crop plants having a modified fatty acid composition (WO 91/13972).

Numerous molecular biological techniques which allow the preparation of novel transgenic plants having modified properties are known in principle; see, for example, Sambrook et al., 1989, Molecular Cloning, A Laboratory Manual, 2nd ed. Cold Spring Harbor Laboratory Press, Cold Spring Harbor, N.Y.; or Winnacker “Gene und Klone” [Genes and Clones], VCH Weinheim, 2nd edition 1996, or Christou, “Trends in Plant Science” 1 (1996) 423-431).

In order to carry out such genetic engineering manipulations, it is possible to introduce nucleic acid molecules into plasmids which allow a mutagenesis or a change in the sequence to occur by recombination of DNA sequences. Using the abovementioned standard processes it is possible, for example, to exchange bases, to remove partial sequences or to add natural or synthetic sequences. To link the DNA fragments -with each other, it is possible to attach adaptors or linkers to the fragments.

Plant cells having a reduced activity of a gene product can be prepared, for example, by expressing at least one appropriate antisense-RNA, a sense-RNA to achieve a cosuppression effect, or by expressing at least one appropriately constructed ribozyme which specifically cleaves transcripts of the abovementioned gene product.

To this end, it is possible to employ both DNA molecules which comprise the entire coding sequence of a gene product, including any flanking sequences that may be present, and DNA molecules which comprise only parts of the coding sequence, it being necessary for these parts to be long enough to cause an antisense effect in the cells. It is also possible to use DNA sequences which have a high degree of homology to the coding sequences of a gene product but which are not entirely identical.

When expressing nucleic acid molecules in plants, the synthesized protein can be localized in any desired compartment of the plant cell. However, to achieve localization in a certain compartment, it is, for example, possible to link the coding region with DNA sequences which ensure localization in a certain compartment. Such sequences are known to the person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated to whole plants using known techniques. The transgenic plants can in principle be plants of any desired plant species, i.e. both monocotyledonous and dicotyledonous plants.

In this manner, it is possible to obtain transgenic plants which have modified properties by overexpression, suppression or inhibition of homologous (=natural) genes or gene sequences or by expression of heterologous (=foreign) genes or gene sequences.

The compounds (I) according to the invention can preferably be used in transgenic crops which are resistant to herbicides selected from the group consisting of the sulfonylureas, glufosinate-ammonium or glyphosate-isopropylammonium and analogous active compounds.

When using the active compounds according to the invention in transgenic crops, in addition to the effects against harmful plants which can be observed in other crops, there are frequently effects which are specific for the application in the respective transgenic crop, for example a modified or specifically broadened spectrum of weeds which can be controlled, modified application rates which can be used for the application, preferably good combinability with the herbicides to which the transgenic crop is resistant, and an effect on the growth and the yield of the transgenic crop plants.

The invention therefore also provides for the use of the compounds (I) according to the invention as herbicides for controlling weed plants in transgenic crop plants.

The compounds according to the invention can be applied in the customary formulations in the form of wettable powders, emulsifiable concentrates, sprayable solutions, dusts or granules. The invention therefore also provides herbicidal and plant growth regulating compositions comprising compounds of the formula (I).

The compounds of the formula (I) can be formulated in various ways depending on the prevailing biological and/or chemico-physical parameters. Examples of suitable formulation options are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing compositions, granules for broadcasting and soil application, granules (GR) in the form of microgranules, spray granules, coating granules and adsorption granules, water-dispersible granules (WG), water-soluble granules (SG), ULV formulations, micro-capsules and waxes.

These individual formulation types are known in principle and are described, for example, in Winnacker-Küchler, “Chemische Technologie” [Chemical Technology], Volume 7, C. Hauser Verlag Munich, 4th edition 1986; Wade van Valkenburg, “Pesticide Formulations”, Marcel Dekker, N.Y., 1973; K. Martens, “Spray Drying” Handbook, 3rd ed. 1979, G. Goodwin Ltd. London.

The necessary formulation auxiliaries, such as inert materials, surfactants, solvents and other additives, are likewise known and are described, for example, in: Watkins, “Handbook of Insecticide Dust Diluents and Carriers”, 2nd ed., Darland Books, Caldwell N.J., H.v. Olphen, “Introduction to Clay Colloid Chemistry”; 2nd ed., J. Wiley & Sons, N.Y.; C. Marsden, “Solvents Guide”; 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesell., Stuttgart 1976; Winnacker-Küchler, “Chemische Technologie” [Chemical Technology], Volume 7, C. Hauser Verlag Munich, 4th edition 1986.

Based on these formulations it is also possible to produce combinations with other pesticidally active substances, for example insecticides, acaricides, herbicides and fungicides, and also with safeners, fertilizers and/or growth regulators, for example in the form of a ready-mix or tank mix.

Wettable powders are preparations which are uniformly dispersible in water and which, in addition to the active compound and as well as a diluent or inert substance, also contain surfactants of ionic and/or nonionic type (wetting agents, dispersants), for example polyethoxylated alkyl phenols, polyethoxylated fatty alcohols, polyethoxylated fatty amines, fatty alcohol polyglycol ethersulfates; alkanesulfonates, alkylbenzenesulfonates, sodium ligninsulfonate, sodium 2,2′-dinaphthylmethane-6,6′-disulfonate, sodium dibutyinaphthalenesulfonate or else sodium oleoylmethyltaurinate. To prepare the wettable powders, the herbicidally active compounds are finely ground, for example in customary apparatuses such as hammer mills, fan mills and air-jet mills, and are mixed simultaneously or subsequently with the formulation auxiliaries.

Emulsifiable concentrates are prepared by dissolving the active compound in an organic solvent, for example butanol, cyclohexanone, dimethylformamide, xylene or else relatively high-boiling aromatics or hydrocarbons or mixtures of the organic solvents, with the addition of one or more surfactants of ionic and/or nonionic type (emulsifiers). Examples of emulsifiers which can be used are calcium alkylarylsulfonates, such as Ca-dodecylbenzenesulfonate, or nonionic emulsifiers, such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters or polyoxyethylene sorbitan esters, for example polyoxyethylene sorbitan fatty acid esters.

Dusts are obtained by grinding the active compound with finely divided solid substances, for example-talc, natural clays, such as kaolin, bentonite and pyrophyllite, or diatomaceous earth.

Suspension concentrates can be water- or oil-based. They can be prepared, for # example, by wet milling using commercially customary bead mills, with or without the addition of surfactants, as already mentioned above, for example, in the case of the other formulation types.

Emulsions, for example oil-in-water emulsions (EW), can be prepared for example by means of stirrers, colloid mills and/or static mixers using aqueous organic solvents and, if desired, surfactants as already mentioned above, for example, in the case of the other formulation types.

Granules can be prepared either by spraying the active compound onto adsorptive, granulated inert material or by applying active-compound concentrates to the surface of carriers such as sand, kaolinites or granulated inert material, by means of adhesive binders, for example polyvinyl alcohol, sodium polyacrylate or else mineral oils. Suitable active compounds can also be granulated in the manner which is customary for the preparation of fertilizer granules, if desired as a mixture with fertilizers.

Water-dispersible granules are generally prepared by the customary processes, such as spray-drying, fluidized-bed granulation, disk granulation, mixing using high-speed mixers, and extrusion without solid inert material. For the preparation of disk, fluidized-bed, extruder and spray granules, see for example processes in “Spray-Drying Handbook” 3rd ed. 1979, G. Goodwin Ltd., London; J. E. Browning, “Agglomeration”, Chemical and Engineering 1967, pages 147 ff; “Perry's Chemical Engineer's Handbook”, 5th ed., McGraw-Hill, New York 1973, pp. 8-57.

For further details on the formulation of crop protection products, see for example G. C. Klingman, “Weed Control as a Science”, John Wiley and Sons., Inc., New York, 1961, pages 81-96 and J. D. Freyer, S. A. Evans, “Weed Control Handbook”, 5th ed., Blackwell Scientific Publications, Oxford, 1968, pages 101-103.

The agrochemical formulations generally contain from 0.1 to 99% by weight, in particular from 0.1 to 95% by weight, of active compound of the formula (I). In wettable powders the concentration of active compound is, for example, from about 10 to 90% by weight, the remainder to 100% by weight consisting of customary formulation constituents. In emulsifiable concentrates the concentration of active compound can be from about 1 to 90%, preferably from 5 to 80%, by weight. Formulations in the form of dusts contain from 1 to 30% by weight of active compound, preferably most commonly from 5 to 20% by weight of active compound, while sprayable solutions contain from about 0.05 to 80%, preferably from 2 to 50%, by weight of active compound. In the case of water-dispersible granules, the content of active compound depends partly on whether the active compound is in liquid or solid form and on the granulation auxiliaries, fillers, etc. that are used. In water-dispersible granules the content of active compound, for example, is between 1 and 95% by weight, preferably between 10 and 80% by weight.

In addition, said formulations of active compound may comprise the tackifiers, wetting agents, dispersants, emulsifiers, penetrants, preservatives, antifreeze agents, solvents, fillers, carriers, colorants, antifoams, evaporation inhibitors and pH and viscosity regulators which are customary in each case.

The compounds of the formula (I) or their salts can be used as such or combined in the form of their preparations (formulations) with other pesticidally active compounds, such as, for example, insecticides, acaricides, nematicides, herbicides, fungicides, safeners, fertilizers and/or growth regulators, for example as finished formulations or tank mixes. Suitable active compounds which can be combined with the active compounds according to the invention in mixed formulations or in a tank mix are, for example, known active compounds, whose effect is based on an inhibition of, for example, acetolactate synthase, acetyl-CoA carboxylase, PS I, PS II, HPPDO, phytoene desaturase, protoporphyrinogen oxidase, glutamine synthetase, cellulose biosynthesis, 5-enolpyruvylshikimate-3-phosphate synthetase. Such compounds, and also other compounds that can be used, with a mechanism of action that is, in some cases, unknown or different, are described, for example, in Weed Research 26, 441-445 (1986), or in “The Pesticide Manual”, 11^{th} edition 1997 (hereafter also abbreviated to “PM”) and 12^{th} edition 2000, The British Crop Protection Council and the Royal Soc. of Chemistry (publisher), and in the literature cited therein. For example, the following active compounds may be mentioned as herbicides which are known from the literature and which can be combined with the compounds of the formula (I) (note: the compounds are either referred to by the “common name” in accordance with the International Organization for Standardization (ISO) or by the, chemical names, if appropriate-together with a customary code number): acetochlor; acifluorfen(-sodium); aclonifen; AKH 7088, i.e. [[[1-[5-[2-chloro-4-(trifluoromethyl)phenoxy]-2-nitrophenyl]-2-methoxyethylidene]amino]oxy]acetic acid and its methyl ester; alachlor; alloxydim(-sodium); ametryn; amicarbazone; amidochlor, amidosulfuron; amitrol; AMS, i.e. ammonium sulfamate; anilofos; asulam; atrazine; azafenidin, azimsulfuron (DPX-A8947); aziprotryn; barban; BAS 516H, i.e. 5-fluoro-2-phenyl-4H-3,1-benzoxazin-4-one; beflubutamide; benazolin(-ethyl); benfluralin; benfuresate; bensulfuron(-methyl); bensulide; bentazone; benzobicyclone, benzofenap; benzofluor; benzoylprop(-ethyl); benzthiazuron; bialaphos; bifenox; bispyribac(-sodium); bromacil; bromobutide; bromofenoxim; bromoxynil; bromuron; buminafos; busoxinone; butachlor; butafenacil, butamifos; butenachlor; buthidazole; butralin; butroxydim, butylate; cafenstrole (CH-900); carbetamide; cafentrazone(-ethyl); caloxydim, CDAA, i.e. 2-chlbro-N,N-di-2-propenylacetamide; CDEC, i.e. 2-chloroallyl diethyldithiocarbamate; chlomethoxyfen; chloramben; chlorazifop-butyl, chlormesulon; chlorbromuron; chlorbufam; chlorfenac; chlorflurecol-methyl; chloridazon; chlorimuron(-ethyl); chlornitrofen; chlorotoluron; chloroxuron; chlorpropham; chlorsulfuron; chlorthal-dimethyl; chlorthiamid; chlortoluron, cinidon(-ethyl and -methyl); cinmethylin; cinosulfuron; clefoxydim, clethodim; clodinafop and its ester derivatives (for example clodinafop-propargyl); clomazone; clomeprop; cloproxydim; clopyralid; clopyrasulfuron(-methyl), cloransulam(-methyl), cumyluron (JC₉₄₀); cyanazine; cycloate; cyclosulfamuron (AC₁₀₄); cycloxydim; cycluron; cyhalofop and its ester derivatives (for example butyl ester, DEH-1 12); cyperquat; cyprazine; cyprazole; daimuron; 2,4-D; 2,4-DB; dalapon; desmedipham; desmetryn; di-allate; dicamba; dichlobenil; dichlorprop; diclofop and its esters such as diclofop-methyl; diclosulam, diethatyl(-ethyl); difenoxuron; difenzoquat; diflufenican; diflufenzopyr, dimefuron; dimepiperate, dimethachlor; dimethametryn; dimethenamid (SAN-582H); dimethazone, dimexyflam, dimethipin; dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-dimethylethyl)-N-methyl-1H-pyrazole-4-carboxamide; endothal; EPTC; esprocarb; ethalfluralin; ethametsulfuron-methyl; ethidimuron; ethiozin; ethofumesate; ethoxyfen and its esters (for example ethyl ester, HN-252); ethoxysulfuron, etobenzanid (HW 52); F5231, i.e. N-[2-chloro-4-fluoro-5-[4-(3-fluoropropyl)-4,5-dihydro-5-oxo-1H-tetrazol-1-yl]-phenyl]ethanesulfonamide; fenoprop; fenoxan, fenoxaprop and fenoxaprop-P and their esters, for example fenoxaprop-P-ethyl and fenoxaprop-ethyl; fenoxydim; fentrazamide; fenuron; flamprop(-methyl or -isopropyl or -isopropyl-L); flazasulfuron; floazulate, florasulam, fluazifop and fluazifop-P and their esters, for example fluazifop-butyl and fluazifop-P-butyl; flucarbazone(-sodium), fluchloralin; flumetsulam; flumeturon; flumiclorac(-pentyl), flumioxazin (S-482); flumipropyn; fluometuron, fluorochloridone, fluorodifen; fluoroglycofen(-ethyl); flupoxam (KNW-739); flupropacil (UBIC-4243); flupyrsulfuron(-methyl, or -sodium), flurenol(-butyl), fluridone; flurochloridone; fluroxypyr(-meptyl); flurprimidol, flurtamone; fluthiacet-(-methyl), fluthiamide, fomesafen; foramsulfuron, fosamine; furyloxyfen; glufosinate(-ammonium); glyphosate(-isopropylammonium); halosafen; halosulfuron(-methyl) and its esters (for example methyl ester, NC-319); haloxyfop and its esters; haloxyfop-P (=R-haloxyfop) and its esters; hexazinone; imazamethabenz(-methyl); imazapyr; imazaquin and salts such as the ammonium salt; imazamethapyr, imazamox, imazapic, imazethamethapyr; imazethapyr, imazosulfuron; indanofan, ioxynil; isocarbamid; isopropalin; isoproturon; isouron; isoxaben; isoxachlortole, isoxaflutole, isoxapyrifop; karbutilate; lactofen; lenacil; linuron; MCPA; MCPB; mecoprop; mefenacet; mefluidid; mesosulfuron, mesotrione, metamitron; metazachlor; methabenzthiazuron; metham; methazole; methoxyphenone; methyidymron; metabenzuron, methobenzuron; metobromuron; (alpha-)metolachlor; metosulam (XRD 511); metoxuron; metribuzin; metsulfuronmethyl; MH; molinate; monalide; monocarbamide dihydrogensulfate; monolinuron; monuron; MT 128, i.e. 6-chloro-N-(3-chloro-2-propenyl)-5-methyl-N-phenyl-3-pyridazinamine; MT 5950, i.e. N-[3-chloro4-(1-methylethyl)-phenyl]-2-methylpentanamide; naproanilide; napropamide; naptalam; NC₃₁₀, i.e. 4-(2,4-dichlorobenzoyl)-1-methyl-5-benzyloxypyrazole; neburon; nicosulfuron; nipyraclophen; nitralin; nitrofen; nitrofluorfen; norflurazon; orbencarb; oryzalin; oxadiargyl (RP-020630); oxadiazone; oxasulfuron; oxaciclomefone; oxyfluorfen; paraquat; pebulate; pelargonic acid, pendimethalin; pentoxazone, perfluidone; phenisopham; phenmedipham; picloram; picolinafen, piperophos; piributicarb; pirifenop-butyl; pretilachlor; primisulfuron(-methyl); procarbazone(-sodium), procyazine; prodiamine; profluralin; proglinazine(-ethyl); prometon; prometryn; propachlor; propanil; propaquizafop and its esters; propazine; propham; propisochlor; propyzamide; prosulfalin; prosulfocarb; prosulfuron (CGA-152005); prynachlor; pyroflufen(-ethyl), pyrazolinate; pyrazon; pyrazosulfuron(-ethyl); pyrazoxyfen; pyribenzoxim, pyributicarb, pyridafol, pyridate; pyriminobac(-methyl), pyrithiobac(-sodium) (KIH-2031); pyroxofop and its esters (for example propargyl ester); quinclorac; quinmerac; quinoclamine, quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; SN 106279, i.e. 2-[[7-[2-chloro-4-(trifluoromethyl)phenoxy]-2-naphthalenyl]oxy]propanoic acid and methyl ester; sulcotrione, sulfentrazone (FMC-97285, F-6285); sulfazurone; sulfometuron-(-methyl); sulfosate (IC1-A0224); sulfosulfuron, TCA; tebutam (GCP-5544); tebuthiuron; tepraloxydim, terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchlor (NSK-850); thiafluamide; thiazafluron; thiazopyr (Mon-13200); thidiazimin (SN-24085); thifensulfuron(-methyl); thiobencarb; tiocarbazil; tralkoxydim; tri-allate; triasulfuron; triaziflam, triazofenamide; tribenuron(-methyl); triclopyr; tridiphane; trietazine; trifluralin; triflusulfuron and esters (e.g. methyl estser, DPX-66037); trimeturon; tritosulfuron, tsitodef; vernolate; WL 110547, i.e. 5-phenoxy-1-[3-(trifluoromethyl)phenyl]-1H-tetrazole; BAY MKH 6561, UBH-509; D489; LS 82-556; KPP-300; NC-324; NC-330; KH-218; DPX-N8189; SC-0774; DOWCO-535; DK-8910; V-53482; PP-600; MBH-001; KIH-9201; ET-751; KIH-6127 and KIH-2023.

Of particular interest is the selective control of harmful plants in crops of useful and ornamental plants. Although the compounds (I) according to the invention have very good to satisfactory selectivity in a large number of crops, it is possible in principle that phytoxicity in the crop plants can occur in some crops and, in particular, when the compounds (I) are mixed with other herbicides which are less selective. In this respect, combinations of the compounds of the formula (I) according to the invention which contain the compounds of the formula (I), or their combinations with other herbicides or pesticides, and safeners are of particular interest. The safeners, which are employed in such amounts that they act as antidotes, reduce the phytotoxic side effects of the herbicides/pesticides used, for example in economically important crops such as cereals (wheat, barley, rye, maize, rice, millet), sugar beet, sugar cane, seed rape, cotton and soya, preferably cereal. Suitable safeners for the compounds (I) and their combinations with other pesticides are, for example, the following groups of compounds:

a) Compounds of the type of dichlorophenylpyrazoline-3-carboxylic acid, preferably compounds such as ethyl 1-(2,4-dichlorophenyl)-5-(ethoxycarbonyl)-5-methyl-2-pyrazoline-3-carboxylate (S1-1) (“mefenpyrdiethyl”, PM, pp. 781-782), and related compounds, as described in WO 91/07874,

b) Derivatives of dichlorophenylpyrazole carboxylic acid, preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S11-4), ethyl 1-(2,4-dichlorophenyl)-5-phenylpyrazole-3-carboxylate (S1-5) and related compounds, as described in EP-A-333 131 and EP-A-269 806.

c) Compounds of the type of the triazolecarboxylic acids, preferably compounds such as fenchlorazole(ethyl ester), i.e. ethyl 1-(2,4-dichlorophenyl)-5-trichloromethyl-(1H)-1,2,4-triazole-3-carboxylate (S1-6) and related compounds as described in EP-A-174 562 and EP-A-346 620.

d) Compounds of the type of the 5-benzyl- or 5-phenyl-2-isoxazoline-3-carboxylic acid, or the 5,5-diphenyl-2-isoxazoline-3-carboxylic acid, preferably compounds such as ethyl 5-(2,4-dichlorobenzyl)-2-isoxazoline-3-carboxylate (S11-7) or ethyl 5-phenyl-2-isoxazoline-3-carboxylate (S1-8) and related compounds, as described in WO 91/08202, or ethyl 5,5-diphenyl-2-isoxazolinecarboxylate (S1-9) (“isoxadifen-ethyl”) or its -n-propyl ester (S1-10) or ethyl 5-(4-fluorophenyl)-5-phenyl-2-isoxazoline-3-carboxylate (S1-11), as described in the German patent application (WO-A-95/07897).

e) Compounds of the type of the 8-quinolineoxyacetic acid (S2), preferably 1-methylhex-1-yl (5-chloro-8-quinolineoxy)acetate (common name “cloquintocet-mexyl” (S2-1) (see PM, pp. 263-264) 1,3-dimethylbut-1-yl (5-chloro-8-quinolineoxy)acetate (S2-2), 4-allyloxybutyl (5-chloro-8-quinolineoxy)acetate (S2-3), 1-allyloxyprop-2-yl (5-chloro-8-quinolineoxy)acetate (S2-4), ethyl (5-chloro-8-quinolineoxy)acetate (S2-5), methyl (5-chloro-8-quinolineoxy)acetate (S2-6), allyl (5-chloro-8-quinolineoxy)acetate (S2-7), 2-(2-propylideneiminoxy)-1-ethyl (5-chloro-8-quinolineoxy)acetate (S2-8), 2-oxo-prop-1-yl (5-chloro-8-quinolineoxy)acetate (S2-9) and related compounds, as described in EP-A-86 750, EP-A-94 349 and EP-A-191 736 or EP-A-0 492 366.

f) Compounds of the type of the (5-chloro-8-quinolineoxy)malonic acid, preferably compounds such as diethyl (5-chloro-8-quinolineoxy)malonate, diallyl (5-chloro-8-quinolineoxy)malonate, methyl ethyl (5-chloro-8-quinolineoxy)malonate and related compounds, as described in EP-A-0 582 198.

g) Active compounds of the type of the phenoxyacetic or -propionic acid derivatives or the aromatic carboxylic acids, such as, for example, 2,4-dichlorophenoxyacetic acid (esters) (2,4-D), 4-chloro-2-methylphenoxypropionic esters (Mecoprop), MCPA or 3,6-dichloro-2-methoxybenzoic acid (esters) (Dicamba).

h) Active compounds of the type of the pyrimidines, which are used as soil-acting safeners in rice, such as, for example, “fenclorim” (PM, pp. 512-511) (=4,6-dichloro-2-phenylpyrimidine), which is known as safenerfor pretilachlor in sown rice,

i) Active compounds of the type of the dichloroacetamides, which are frequently used as pre-emergent safeners (soil-acting safeners), such as, for example,

“dichlormid” (PM, pp. 363-364) (=N,N-diallyl-2,2-dichloroacetamide),

“R-29148” (=3-dichloroacetyl-2,2,5-trimethyl-1,3-oxazolidine from Stauffer),

“benoxacor” (PM, pp.102-103) (=4-dichloroacetyl-3,4-dihydro-3-methyl-2H-1,4-benzoxazine),

“PPG-1292” (=N-allyl-N-[(1,3-dioxolan-2-yl)methyl]dichloroacetamide from PPG Industries),

“DK-24” (=N-allyl-N-[(allylaminocarbonyl)methyl]dichloroacetamide from Sagro-Chem),

“AD-67” or “MON 4660” (=3-dichloroacetyl-1-oxa-3-aza-spiro[4,5]decane from Nitrokemia or Monsanto),

“diclonon” or “BAS145138” or “LAB145138” (=3-dichloroacetyl-2,5,5-trimethyl-1,3-diazabicyclo[4.3.0]nonane from BASF) and

“furilazol” or “MON 13900” (see PM, 637-638) (=(RS)-3-dichloroacetyl-5-(2-furyl)-2,2-dimethyloxazolidine)

j) Active compounds of the type of the dichloroacetone derivatives, such as, for example,

“MG 191” (CAS-Reg. No. 96420-72-3) (=2-dichloromethyl-2-methyl-1,3-dioxolane from Nitrokemia), which is known as safener for maize,

k) Active compounds of the type of the oxyimino compounds, which are known as seed dressings, such as, for example,

“oxabetrinil” (PM, pp. 902-903) (=(Z)-1,3-dioxolan-2-ylmethoxyimino(phenyl)acetonitrile), which is known as seed dressing safener for millet against metolachlor damage,

“fluxofenim” (PM, pp. 613-614) (=1-(4-chlorophenyl)-2,2,2-trifluoro-1-ethanone O-(1,3-dioxolan-2-ylmethyl) oxime), which is known as seed dressing safener for millet against metolachlor damage,

“cyometrinil” or “-CGA-43089” (PM, p. 1304) (=(Z)-cyanomethoxyimino-(phenyl)acetonitrile), which is known as seed dressing safener for millet against metolachlor damage,

l) Active compounds of the type of the thiazolecarboxylic esters, which are known as seed dressings, such as, for example,

“flurazol” (PM, pp. 590-591) (=benzyl 2-chloro-4-trifluoromethyl-1,3-thiazole-5-carboxylate), which is known as seed dressing safener for millet against alachlor and metolachlor damage,

m) Active compounds of the type of the naphthalenedicarboxylic acid derivatives, which are known as seed dressings, such as, for example,

“naphthalic anhydride” (PM, p.1342) (=1,8-naphthalenedicarboxylic anhydride), which is known as seed dressing safener for maize against thiocarbamate herbicide damage,

n) Active compounds of the type of the chromanacetic acid derivatives, such as, for example,

“CL 304415” (CAS-Reg. No. 31541-57-8) (=2-(4-carboxychroman-4-yl)acetic acid from American Cyanamid), which is known as safener for maize against imidazolinone damage,

o) Active compounds which, in addition to a herbidical action against harmful plants, also havesafener action in crop plants such as rice, such as, for example,

“dimepiperate” or “MY-93” (PM, pp. 404405) (=S-1-methyl-1-phenylethyl piperidine-1-thiocarboxylate), which is known as safener for rice against herbicide molinate damage,

“daimuron” or “SK 23” (PM, p. 330) (=1-(1-methyl-1-phenylethyl)-3-p-tolylurea), which is known as safener for rice against herbicide imazosulfuron damage,

“cumyluron”=“JC-940” (=3-(2-chlorophenylmethyl)-1-(1-methyl-1-phenylethyl)urea, see JP-A-60087254), which is known as safener for rice against damage by some herbicides,

“methoxyphenon” or “NK 049” (=3,3′-dimethyl-4-methoxy-benzophenone), which is known as safener for rice against damage by some herbicides,

“CSB” (=1-bromo-4-(chloromethylsulfonyl)benzene) (CAS-Reg. No. 54091-06-4 from Kumiai), which is known as safener against damage by some herbicides in rice

p) N-Acylsulfonamides of the formula (S3) and salts thereof,

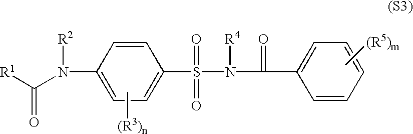

as described in WO-A-97/45016,

q) Acylsulfamoylbenzoamides of the formula (S4), if appropriate also in salt form,

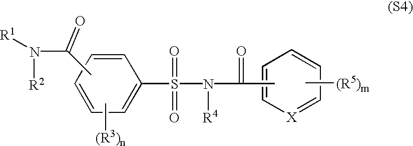

as described in the International Application No. PCT/EP98/06097, and

r) compounds of the formula (S5),

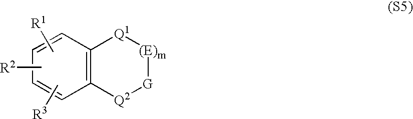

as described in WO-A 98/13 361,

including the stereoisomers and the salts used in agriculture.

Among the safeners mentioned, (S1-1) and (S1-9) and (S2-1), in particular (S1-1) and (S1-9), are of particular interest.

Some of the safeners are already known as herbicides and consequently show, in addition to the herbicidal action against harmful plants, also protective action in connection with crop plants.

The ratios by weight of herbicide (mixture) to safener generally depend on the application rate of the herbicide and the efficacy of the safener in question and can vary within wide limits, for example in the range from 200:1 to 1:200, preferably 100:1 to 1:100, in particular 20:1 to 1:20. Analogously to the compounds of the formula (I) or their mixtures, the safeners can be formulated with other herbicides/pesticides and can be provided and used as ready-mixes or tank mixes with the herbicides.

For use, the formulations which are present in commercially available form are, if appropriate, diluted in the customary manner, for example using water in the case of wettable powders, emulsifiable concentrates, dispersions and water-dispersible granules. Preparations in the form of dusts, granules for soil application or broadcasting and sprayable solutions are usually not further diluted with other inert substances prior to use.

The application rate of the compounds of the formula (I) required varies with the external conditions, such as temperature, humidity, the nature of the herbicide used and the like. It can vary within wide limits, for example between 0.001 and 10.0 kg/ha or more of active substance, but it is preferably between 0.005 and 5 kg/ha.

In the examples below, the amounts (including percentages) are by weight, unless specifically indicated otherwise.

### SYNTHESIS EXAMPLES

In the text below, a number of synthesis examples are given by way of example, and describe the preparation of certain synthesis precursors or salts thereof (A1-A5) of compounds of the formula (I) and also the synthesis of certain compounds (B1-B4) of the formula (I) or salts thereof.

### A. Preparation Examples for Synthesis Precursors

### Example A1

3-Hydroxypyrrolidin-2-ones

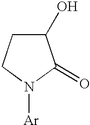

3-Hydroxypyrrolidin-2-ones may be prepared conventionally by condensing α-hydroxy-γ-butyrolactones with correspondingly substituted aromatic amines without solvent [e.g.: Seidel, M. C.; J. Heterocycl. Chem. 3(3) 355-356 (1966)]. An alternative variant is the reaction of the correspondingly substituted aromatic amine with commercial 2,4-dibromobutyryl chloride, followed by substitution of the bromine atom in position 3 by acetate, followed by basic hydrolysis of the acetate [e.g.: Marinelli, E. R.; Arunachalam, T.; Diamantidis, G.; Emswiler, J.; Fan, H.; Tetrahedron 52(34) 11177-11214 (1996)].

### Example A2

3-Aminopyrrolidin-2-ones

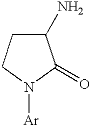

3-Aminopyrrolidin-2-ones may be prepared conventionally by condensing α-amino-γ-butyrolactone hydrobromide with correspondingly substituted aromatic amines without solvent [e.g.: Okumura, K.; Inoue, I.; Ikezaki, M.; Hayashi, G.; Nurimoto, S.; Shintomi, K.; J. Med. Chem 9 315-319 (1966)].

An alternative variant is the reaction of the correspondingly substituted aromatic amine with commercial 2,4-dibromobutyryl chloride, followed by substitution of the bromine atom in position 3 by aqueous ammonia solution [e.g.: Colson, P.-J.; Przybyla, C. A.; Wise, B. E.; Babiak, K. A.; Seaney, L. M.; Korte, D. E.; Tetrahedron: Asymmetry 9(15) 2587-2594 (1998)].

### Example A3

3-(Methylamino)pyrrolidin-2-ones

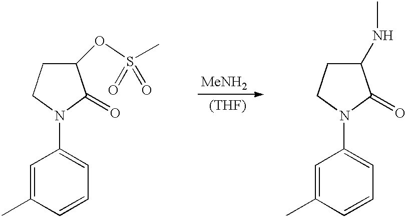

2.3 g (7.1 mmol) of 1-(3-methylphenyl)-3-methylsulfonyloxy-2-pyrrolidinone were dissolved in 60 ml of tetrahydrofuran and the solution was cooled to 0° C. Methylamine gas was introduced until the solution was saturated, the temperature was allowed to rise to 25° C., further methylamine was introduced to saturation, and the solution was left to stand for 15 hours. The reaction solution was concentrated, the residue was taken up in ethyl acetate and the organic phase was washed with water and saturated sodium chloride solution. It was then dried over magnesium sulphate. This gave 1.8 g of the title compound with a melting point of 76° C.

### Example A4

3-Mercaptopyrrolidin-2-ones

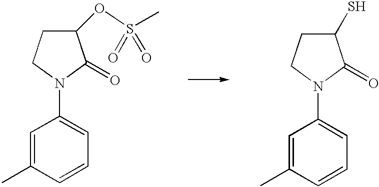

3-Mercaptopyrrolidin-2-ones may be prepared, for example, by reacting a mesylate with potassium thioacetate in dimethylformamide and then hydrolyzing the acetyl group with, for example, sodium hydroxide in methanol [e.g.: Oh, C.-H.; Lee, S. C.; Park, S.-J.; Lee, I.-K.; Nam, K. H.; Lee, K.-S.; Chung, B.-Y.; Cho, J.-H.; Arch. Pharm. 332 (4)111-114 (1999)].

One alternative is to react a tosylate with thiourea in pyridine, followed by basic hydrolysis with sodium hydroxide, for example [e.g.: Blanco, J. M.; Caamano, O.; Eirin, A.; Medina, L.; Fernandez, F.; Synthesis 7 584-586 (1990)].

Linking to the aromatic moieties takes place in analogy to the synthesis examples described.

### Example A5

3-Substituted Pyrrolidines

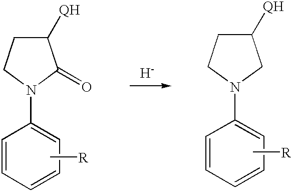

Pyrrolidines are obtained prior to linking to the aromatic moieties from the corresponding pyrrolidin-2-ones by reduction with appropriate reducing agents such as, for example, lithium aluminum hydride in an appropriate solvent such as diethyl ether or tetrahydrofuran, for example [e.g.: Arakawa, Y.; Yoshifuji, S. Chem. Pharm. Bull. 39(9) 2219-2224 (1991)].

Linking to the aromatic moieties takes place in analogy to the synthesis examples described.

### B. Preparation Examples for Compounds of the Formula (I)

### Example B1

Pyrrolidine-2-thiones

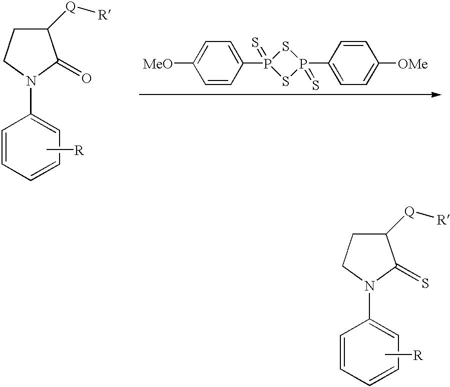

For preparing pyrrolidine-2-thiones, the pyrrolidinones (already substituted) are treated with Lawson's reagent [2,4-bis(4-methylphenylthio)-1,3-dithia-2,4-diphosphetane-2,4-dithione] in toluene [e.g.: Wipf, P.; Jenni, C.; Heimgartner, H. Helv. Chim. Acta 70(4) 1001 (1987)].

### Example B2

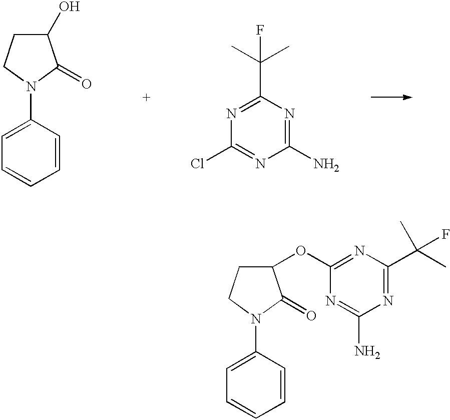

300 mg (1.7 mmol) of N-phenyl-3-hydroxypyrrolidin-2-one were introduced as an initial charge in 10 ml of DMF, a total of 56 mg (1.9 mmol) of 80% sodium hydride were added in portions at room temperature, and the mixture was left with stirring at room temperature for 15 minutes. Then 355 mg (1.9 mmol) of chloroaminotriazine (see above) were added in portions and stirring was continued for 1 hour. The reaction mixture was admixed with water and extracted with ethyl acetate. The organic phase was washed with water, dried over MgSO₄ and concentrated.

Chromatography on silica gel using n-heptane/ethyl acetate (up to 70% ethyl acetate) gave 390 mg of a solid having a melting point of 77° C.

NMR data obtained (instrument used: Mercury 300 MHz; manufacturer: Varian; year of construction: 1997)

(CDCl₃, 300 MHz): δ [ppm]=7.65 (d, 2H); 7.40 (tr, 2H); 7.20 (tr, 1H); 5.90 (tr, 1H); 3.90 (m, 2H); 2.75 (m, 1H); 2.35 (m, 1H); 1.70 (d, 6H)

### Example B3

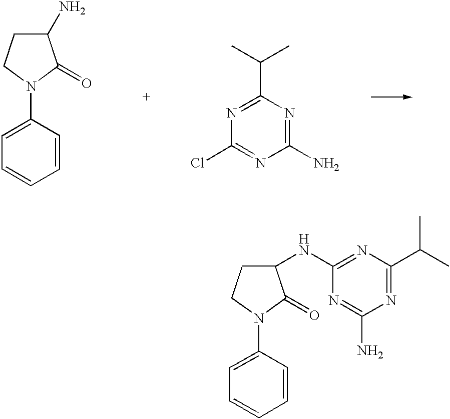

300 mg (1.7 mmol) of N-phenyl-3-aminopyrrolidon-2-one were refluxed together with 323 mg (1.9 mmol) of chloroaminotriazine (see above) and 588 mg (4.3 mmol) of potassium carbonate in 5 ml of acetonitrile for 3 hours. After cooling to 25° C., ether was added, the mixture was washed with water, and the ethereal phase was dried over magnesium sulfate and concentrated. Chromatography on silica gel using ethyl acetate gave 180 mg of a white solid having a melting point of 210° C.

NMR data obtained (instrument used: Mercury 300 MHz; manufacturer: Varian; year of construction: 1997)

(CDCl₃, 300 MHz): δ [ppm]=7.65 (d, 2H); 7.40 (tr, 2H); 7.20 (tr, 1H); 5.80 (dbr, 1H); 5.00 (sbr, 2H); 4.70 (m, 1H); 3.85 (m, 2H); 2.90 (m, 1H); 2.70 (sept, 1H); 2.05 (m, 1H); 1.20 (d, 6H).

### Example B4

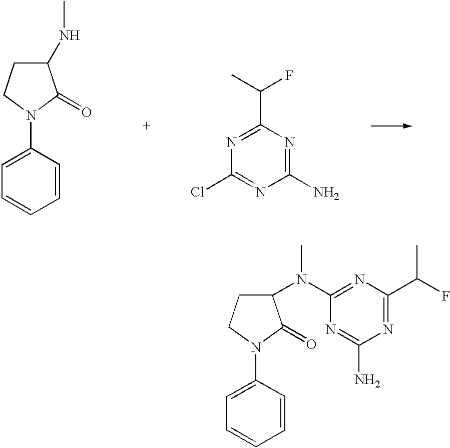

300 mg (1.5 mmol) of N-(3-methylphenyl)-3-(aminomethyl)pyrrolidin-2-one were refluxed with 285 mg (1.6 mmol) of chloroaminotriazine (see above) and 507 mg (3.7 mmol) of potassium carbonate in 5 ml of acetonitrile for 2 hours.

After cooling to 25° C., the mixture was filtered over silica gel together with a mixture of 20% n-heptane and 80% ethyl acetate. This gave 270 mg of a white solid having a melting point of 206° C.

NMR data obtained (instrument used: Mercury 300 MHz; manufacturer: Varian; production number CO10270; year of construction: 1997)

(CDCl₃, 300 MHz): δ [ppm]=7.50 (s, 1H); 7.40 (d, 1H); 7.25 (tr, 1H); 7.00 (d, 1H); 5.60 (m, 1H); 5.20 (m, 1H); 5.10 (m, 1H); 3.85 (m, 2H); 3.20 (m, 3H); 2.45 (m, 1H); 2.40 (s, 3H); 2.30 (m, 1H); 1.60 (m, 3H)

The exemplary compounds set out in tables A to H below are obtained in analogy to examples A1-A5 and, respectively, B1 to B4 and the processes referred to generally in the description.

Abbreviations:

Me=Methyl

Ex. No. =Example number of a compound

c-Pr=Cyclopropyl—

—=single bond

The bonding site of the cyclic radicals is indicated by a bond line (not to be confused with the otherwise customary abbreviated way of writing a methyl radical, which is not used in these tables).

TABLE A

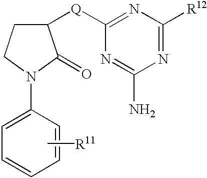

| Ex. | | | |
|---|---|---|---|
| No. | Q | R¹¹ | R¹² |
| A1 | NH | 3-F | Me |
| A2 | NH | H | Me |
| A3 | NH | 3-CF₃ | Me |
| A4 | NH | 3-Me | Me |
| A5 | NH | 3,5-Me₂ | Me |
| A6 | NH | 3-Me-4-F | Me |
| A7 | NH | 3-OMe | Me |
| A8 | NH | 3-OCF₃ | Me |
| A9 | NH | 3-Cl | Me |
| A10 | NH | 3-F | CHFCH₃ |
| A11 | NH | H | CHFCH₃ |
| A12 | NH | 3-CF₃ | CHFCH₃ |
| A13 | NH | 3-Me | CHFCH₃ |
| A14 | NH | 3,5-Me₂ | CHFCH₃ |
| A15 | NH | 3-Me-4-F | CHFCH₃ |
| A16 | NH | 3-OMe | CHFCH₃ |
| A17 | NH | 3-OCF₃ | CHFCH₃ |
| A18 | NH | 3-Cl | CHFCH₃ |
| A19 | NH | 3-F | CH(CH₃)₂ |
| A20 | NH | H | CH(CH₃)₂ |
| A21 | NH | 3-CF₃ | CH(CH₃)₂ |
| A22 | NH | 3-Me | CH(CH₃)₂ |
| A23 | NH | 3,5-Me₂ | CH(CH₃)₂ |
| A24 | NH | 3-Me-4-F | CH(CH₃)₂ |
| A25 | NH | 3-OMe | CH(CH₃)₂ |
| A26 | NH | 3-OCF₃ | CH(CH₃)₂ |
| A27 | NH | 3-Cl | CH(CH₃)₂ |
| A28 | NH | 3-F | CF(CH₃)₂ |
| A29 | NH | 3-Me-4-F | CF(CH₃)₂ |
| A30 | NH | 3-OMe | CF(CH₃)₂ |
| A31 | NH | 3-OCF₃ | CF(CH₃)₂ |
| A32 | NH | 3-CF₃ | CF(CH₃)₂ |
| A33 | NH | 3-Me | CF(CH₃)₂ |
| A34 | NH | 3,5-Me₂ | CF(CH₃)₂ |
| A35 | NH | H | CF(CH₃)₂ |
| A36 | NH | 3-Cl | CF(CH₃)₂ |
| A37 | O | 3-CF₃ | CHFCH₃ |
| A38 | O | 3-Me | CHFCH₃ |
| A39 | O | 3,5-Me₂ | CHFCH₃ |
| A40 | O | 3-Me-4-F | CHFCH₃ |
| A41 | O | 3-OMe | CHFCH₃ |
| A42 | O | 3-OCF₃ | CHFCH₃ |
| A43 | O | 3-Cl | CHFCH₃ |
| A44 | O | 3-F | CHFCH₃ |
| A45 | O | H | CH(CH₃)₂ |
| A46 | O | 3-CF₃ | CH(CH₃)₂ |
| A47 | O | 3-Me | CH(CH₃)₂ |
| A48 | O | 3,5-Me₂ | CH(CH₃)₂ |
| A49 | O | 3-Me-4-F | CH(CH₃)₂ |
| A50 | O | 3-OMe | CH(CH₃)₂ |
| A51 | O | 3-OCF₃ | CH(CH₃)₂ |
| A52 | O | 3-Cl | CH(CH₃)₂ |
| A53 | O | 3-F | CH(CH₃)₂ |
| A54 | O | 3-CF₃ | CF(CH₃)₂ |
| A55 | O | H | CF(CH₃)₂ |
| A56 | O | 3-Me | CF(CH₃)₂ |
| A57 | O | 3,5-Me₂ | CF(CH₃)₂ |
| A58 | O | 3-Me-4-F | CF(CH₃)₂ |
| A59 | O | 3-OMe | CF(CH₃)₂ |
| A60 | O | 3-OCF₃ | CF(CH₃)₂ |
| A61 | O | 3-Cl | CF(CH3)2 |
| A62 | O | 3-F | CF(CH3)2 |
| A63 | NMe | 3-OCF₃ | CHFCH₃ |
| A64 | NMe | 3-CF₃ | CHFCH₃ |
| A65 | NMe | 3,5-Me₂ | CHFCH₃ |
| A66 | NMe | 3-Cl | CHFCH₃ |
| A67 | NMe | 3-Me-4-F | CHFCH₃ |
| A68 | NMe | 3-Me | CHFCH₃ |
| A69 | NMe | 3-F | CHFCH₃ |
| A70 | NMe | 3-CF₃ | CH(CH₃)₂ |
| A71 | NMe | 3,5-Me₂ | CH(CH₃)₂ |
| A72 | NMe | 3-Cl | CH(CH₃)₂ |
| A73 | NMe | 3-Me-4-F | CH(CH₃)₂ |
| A74 | NMe | 3-Me | CH(CH₃)₂ |
| A75 | NMe | 3-OCF₃ | CH(CH₃)₂ |
| A76 | NMe | 3-F | CH(CH₃)₂ |
| A77 | NMe | 3-OMe | CF(CH₃)₂ |
| A78 | NMe | 3,5-Me₂ | CF(CH₃)₂ |
| A79 | NMe | 3-Cl | CF(CH₃)₂ |
| A80 | NMe | 3-OCF₃ | CF(CH₃)₂ |
| A81 | NMe | 3-CF₃ | CF(CH₃)₂ |
| A82 | NMe | 3-F | CF(CH₃)₂ |
| A83 | NMe | 3-Me-4-F | CF(CH₃)₂ |
| A84 | NMe | 3-Me | CF(CH₃)₂ |

TABLE B

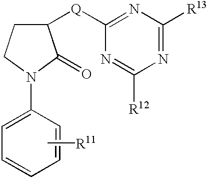

| Ex. | | | | |
|---|---|---|---|---|
| No. | Q | R¹¹ | R¹² | R¹³ |
| B1 | O | 3-CF₃ | CF(CH₃)₂ | NHMe |
| B2 | O | 3-OCF₃ | CF(CH₃)₂ | NHMe |
| B3 | O | 3,5-Me₂ | CF(CH₃)₂ | NHMe |
| B4 | O | 3-Cl | CF(CH₃)₂ | NHMe |
| B5 | NH | 3-CF₃ | CF(CH₃)₂ | NHMe |
| B6 | NH | 3-OCF₃ | CF(CH₃)₂ | NHMe |
| B7 | NH | 3,5-Me₂ | CF(CH₃)₂ | NHMe |
| B8 | NH | 3-Cl | CF(CH₃)₂ | NHMe |
| B9 | NMe | 3-CF₃ | CF(CH₃)₂ | NHMe |
| B10 | NMe | 3-OCF₃ | CF(CH₃)₂ | NHMe |
| B11 | NMe | 3,5-Me₂ | CF(CH₃)₂ | NHMe |
| B12 | NMe | 3-Cl | CF(CH₃)₂ | NHMe |
| B13 | O | 3-CF₃ | CF(CH₃)₂ | NMe₂ |
| B14 | O | 3-OCF₃ | CF(CH₃)₂ | NMe₂ |
| B15 | O | 3,5-Me₂ | CF(CH₃)₂ | NMe₂ |
| B16 | O | 3-Cl | CF(CH₃)₂ | NMe₂ |
| B17 | NH | 3-CF₃ | CF(CH₃)₂ | NMe₂ |
| B18 | NH | 3-OCF₃ | CF(CH₃)₂ | NMe₂ |
| B19 | NH | 3,5-Me₂ | CF(CH₃)₂ | NMe₂ |
| B20 | NH | 3-Cl | CF(CH₃)₂ | NMe₂ |
| B21 | NMe | 3-CF₃ | CF(CH₃)₂ | NMe₂ |
| B22 | NMe | 3-OCF₃ | CF(CH₃)₂ | NMe₂ |
| B23 | NMe | 3,5-Me₂ | CF(CH₃)₂ | NMe₂ |
| B24 | NMe | 3-Cl | CF(CH₃)₂ | NMe₂ |
| B25 | O | 3-CF₃ | CF(CH₃)₂ | NH-c-Pr |
| B26 | O | 3-OCF₃ | CF(CH₃)₂ | NH-c-Pr |
| B27 | O | 3,5-Me₂ | CF(CH₃)₂ | NH-c-Pr |
| B28 | O | 3-Cl | CF(CH₃)₂ | NH-c-Pr |
| B29 | NH | 3-CF₃ | CF(CH₃)₂ | NH-c-Pr |
| B30 | NH | 3-OCF₃ | CF(CH₃)₂ | NH-c-Pr |
| B31 | NH | 3,5-Me₂ | CF(CH₃)₂ | NH-c-Pr |
| B32 | NH | 3-Cl | CF(CH₃)₂ | NH-c-Pr |
| B33 | NMe | 3-CF₃ | CF(CH₃)₂ | NH-c-Pr |
| B34 | NMe | 3-OCF₃ | CF(CH₃)₂ | NH-c-Pr |
| B35 | NMe | 3,5-Me₂ | CF(CH₃)₂ | NH-c-Pr |
| B36 | NMe | 3-Cl | CF(CH₃)₂ | NH-c-Pr |
| B37 | O | 3-CF₃ | OCH₃ | NH₂ |
| B38 | O | 3-OCF₃ | OCH₃ | NH₂ |
| B39 | O | 3,5-Me₂ | OCH₃ | NH₂ |
| B40 | O | 3-Cl | OCH₃ | NH₂ |
| B41 | NH | 3-CF₃ | OCH₃ | NH₂ |
| B42 | NH | 3-OCF₃ | OCH₃ | NH₂ |
| B43 | NH | 3,5-Me₂ | OCH₃ | NH₂ |
| B44 | NH | 3-Cl | OCH₃ | NH₂ |
| B45 | NMe | 3-CF₃ | OCH₃ | NH₂ |
| B46 | NMe | 3-OCF₃ | OCH₃ | NH₂ |
| B47 | NMe | 3,5-Me₂ | OCH₃ | NH₂ |
| B48 | NMe | 3-Cl | OCH₃ | NH₂ |
| B49 | O | 3-CF₃ | SCH₃ | NH₂ |
| B50 | O | 3-OCF₃ | SCH₃ | NH₂ |
| B51 | O | 3,5-Me₂ | SCH₃ | NH₂ |
| B52 | O | 3-Cl | SCH₃ | NH₂ |
| B53 | NH | 3-CF₃ | SCH₃ | NH₂ |
| B54 | NH | 3-OCF₃ | SCH₃ | NH₂ |
| B55 | NH | 3,5-Me₂ | SCH₃ | NH₂ |
| B56 | NH | 3-Cl | SCH₃ | NH₂ |
| B57 | NMe | 3-CF₃ | SCH₃ | NH₂ |
| B58 | NMe | 3-OCF₃ | SCH₃ | NH₂ |
| B59 | NMe | 3,5-Me₂ | SCH₃ | NH₂ |
| B60 | NMe | 3-Cl | SCH₃ | NH₂ |
| B61 | O | 3-CF₃ | NMe₂ | Cl |
| B62 | O | 3-OCF₃ | NMe₂ | Cl |
| B63 | O | 3,5-Me₂ | NMe₂ | Cl |
| B64 | O | 3-Cl | NMe₂ | Cl |
| B65 | NH | 3-CF₃ | NMe₂ | Cl |
| B66 | NH | 3-OCF₃ | NMe₂ | Cl |
| B67 | NH | 3,5-Me₂ | NMe₂ | Cl |
| B68 | NH | 3-Cl | NMe₂ | Cl |
| B69 | NMe | 3-CF₃ | NMe₂ | Cl |
| B70 | NMe | 3-OCF₃ | NMe₂ | Cl |
| B71 | NMe | 3,5-Me₂ | NMe₂ | Cl |
| B72 | NMe | 3-Cl | NMe₂ | Cl |

TABLE C

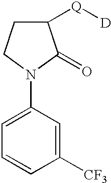

| Ex. | | |
|---|---|---|
| No. | Q | D |
| C1 | O | 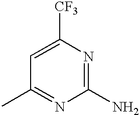 |
| C2 | NH | 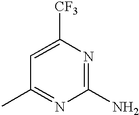 |
| C3 | NMe | 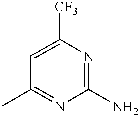 |
| C4 | O | 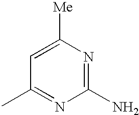 |
| C5 | NH | 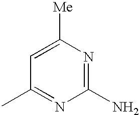 |
| C6 | NMe | 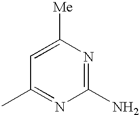 |
| C7 | O | 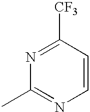 |
| C8 | NH | 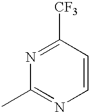 |
| C9 | NMe | 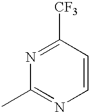 |
| C10 | O | 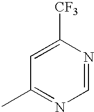 |
| C11 | NH | 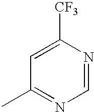 |
| C12 | NMe | 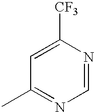 |
| C13 | O | 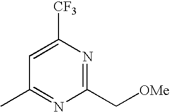 |
| C14 | NH | 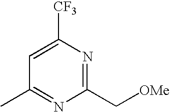 |
| C15 | NMe | 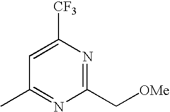 |
| C16 | O | 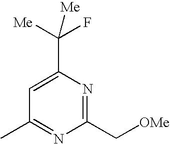 |
| C17 | NH | 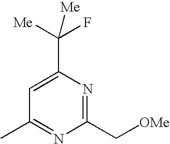 |
| C18 | NMe | 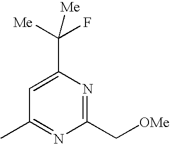 |
| C19 | O | 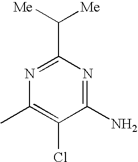 |
| C20 | NH | 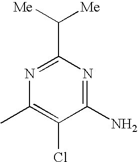 |
| C21 | NMe | 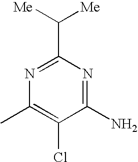 |
| C22 | O | 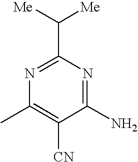 |
| C23 | NH | 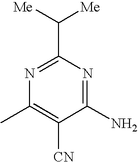 |
| C24 | NMe | 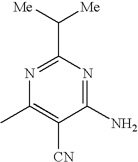 |
| C25 | O | 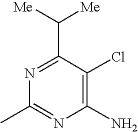 |
| C26 | NH | 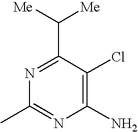 |
| C27 | NMe | 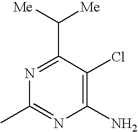 |
| C28 | O | 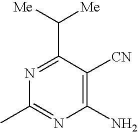 |
| C29 | NH | 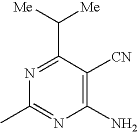 |
| C30 | NMe | 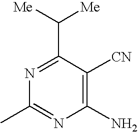 |
| C31 | O | 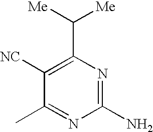 |
| C32 | NH | 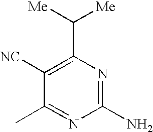 |
| C33 | NMe | 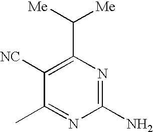 |
| C34 | O | 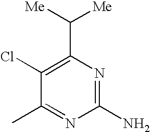 |
| C35 | NH | 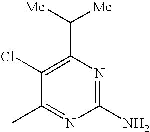 |
| C36 | NMe | 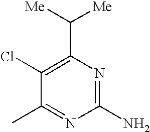 |
| C37 | O | 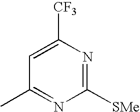 |
| C38 | NH | 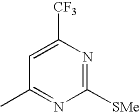 |
| C39 | NMe | 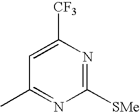 |
| C40 | O | 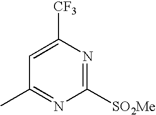 |
| C41 | NH | 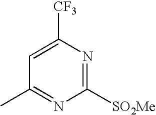 |
| C42 | NMe | 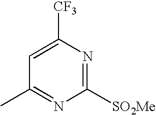 |
| C43 | O | 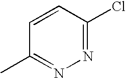 |
| C44 | NH | 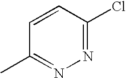 |
| C45 | NMe | 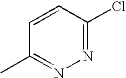 |
| C46 | O | 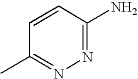 |
| C47 | NH | 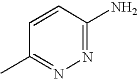 |
| C48 | NMe | 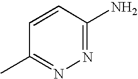 |
| C49 | O | 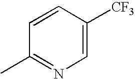 |
| C50 | NH | 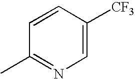 |
| C51 | NMe | 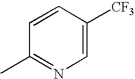 |
| C52 | O | 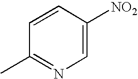 |
| C53 | NH | 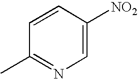 |
| C54 | NMe | 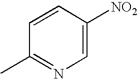 |
| C55 | O | 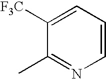 |
| C56 | NH | 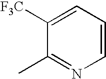 |
| C57 | NMe | 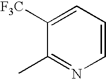 |
| C58 | O | 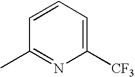 |
| C59 | NH | 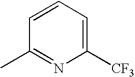 |
| C60 | NMe | 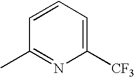 |
| C61 | O | 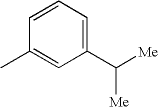 |
| C62 | NH | 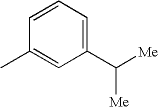 |
| C63 | NMe | 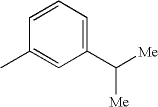 |

TABLE D

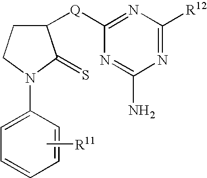

| Ex. | | | |
|---|---|---|---|
| No. | Q | R¹¹ | R¹² |
| D1 | O | 3-CF₃ | CF(CH₃)₂ |
| D2 | O | 3-OCF₃ | CF(CH₃)₂ |
| D3 | O | 3,5-Me₂ | CF(CH₃)₂ |
| D4 | O | 3-Cl | CF(CH₃)₂ |
| D5 | NH | 3-CF₃ | CF(CH₃)₂ |
| D6 | NH | 3-OCF₃ | CF(CH₃)₂ |
| D7 | NH | 3,5-Me₂ | CF(CH₃)₂ |
| D8 | NH | 3-Cl | CF(CH₃)₂ |
| D9 | NMe | 3-CF₃ | CF(CH₃)₂ |
| D10 | NMe | 3-OCF₃ | CF(CH₃)₂ |
| D11 | NMe | 3,5-Me₂ | CF(CH₃)₂ |
| D12 | NMe | 3-Cl | CF(CH₃)₂ |
| D13 | O | 3-CF₃ | CH(CH₃)₂ |
| D14 | O | 3-OCF₃ | CH(CH₃)₂ |
| D15 | O | 3,5-Me₂ | CH(CH₃)₂ |
| D16 | O | 3-Cl | CH(CH₃)₂ |
| D17 | NH | 3-CF₃ | CH(CH₃)₂ |
| D18 | NH | 3-OCF₃ | CH(CH₃)₂ |
| D19 | NH | 3,5-Me₂ | CH(CH₃)₂ |
| D20 | NH | 3-Cl | CH(CH₃)₂ |
| D21 | NMe | 3-CF₃ | CH(CH₃)₂ |
| D22 | NMe | 3-OCF₃ | CH(CH₃)₂ |
| D23 | NMe | 3,5-Me₂ | CH(CH₃)₂ |
| D24 | NMe | 3-Cl | CH(CH₃)₂ |

TABLE E

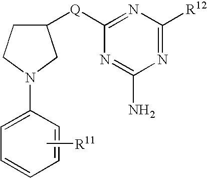

| Ex. | | | |
|---|---|---|---|
| No. | Q | R¹¹ | R¹² |
| E1 | O | 3-CF₃ | CF(CH₃)₂ |
| E2 | O | 3-OCF₃ | CF(CH₃)₂ |
| E3 | O | 3,5-Me₂ | CF(CH₃)₂ |
| E4 | O | p-F-3-Me | CF(CH₃)₂ |
| E5 | O | H | CF(CH₃)₂ |
| E6 | NH | H | CF(CH₃)₂ |
| E7 | NH | 3-Me | CF(CH₃)₂ |
| E8 | NH | 3-Me | CHFCH₃ |
| E9 | NH | 3-Me | CH(CH₃)₂ |
| E10 | NH | 3-Me-4-F | CF(CH₃)₂ |
| E11 | NH | 3-Me-4-F | CHFCH₃ |
| E12 | NH | 3-Me-4-F | CH(CH₃)₂ |
| E13 | NH | 3-F | CF(CH₃)₂ |
| E14 | NH | 3-F | CHFCH₃ |
| E15 | NH | 3-F | CH(CH₃)₂ |
| E16 | NH | 3-Cl | CF(CH₃)₂ |
| E17 | NH | 3-Cl | CHFCH₃ |
| E18 | NH | 3-Cl | CH(CH₃)₂ |
| E19 | NH | 3-CF₃ | CF(CH₃)₂ |
| E20 | NH | 3-CF₃ | CHFCH₃ |
| E21 | NH | 3-CF₃ | CH(CH₃)₂ |
| E22 | NH | 3-OCF₃ | CF(CH₃)₂ |
| E23 | NH | 3-OCF₃ | CHFCH₃ |
| E24 | NH | 3-OCF₃ | CH(CH₃)₂ |
| E25 | NH | 3,5-(CH₃)₂ | CF(CH₃)₂ |
| E26 | NH | 3,5-(CH₃)₂ | CHFCH₃ |
| E27 | NH | 3,5-(CH₃)₂ | CH(CH₃)₂ |
| E28 | NMe | 3-CF₃ | CF(CH₃)₂ |
| E29 | NMe | 3-OCF₃ | CF(CH₃)₂ |
| E30 | NMe | 3,5-Me₂ | CF(CH₃)₂ |
| E31 | NMe | 4-F-3-Me | CF(CH₃)₂ |
| E32 | NMe | H | CF(CH₃)₂ |
| E33 | O | 3-CF₃ | CH(CH₃)₂ |
| E34 | O | 3-OCF₃ | CH(CH₃)₂ |
| E35 | O | 3,5-Me₂ | CH(CH₃)₂ |
| E36 | O | 4-F-3-Me | CH(CH₃)₂ |
| E37 | O | H | CH(CH₃)₂ |
| E38 | NH | 3-CF₃ | CH(CH₃)₂ |
| E39 | NH | 3-OCF₃ | CH(CH₃)₂ |
| E40 | NH | 3,5-Me₂ | CH(CH₃)₂ |
| E41 | NH | 4-F-3-Me | CH(CH₃)₂ |
| E42 | NH | H | CH(CH₃)₂ |
| E43 | NMe | 3-CF₃ | CH(CH₃)₂ |
| E44 | NMe | 3-OCF₃ | CH(CH₃)₂ |
| E45 | NMe | 3,5-Me₂ | CH(CH₃)₂ |
| E46 | NMe | 4-F-3-Me | CH(CH₃)₂ |
| E47 | NMe | H | CH(CH₃)₂ |
| E48 | NMe | 3-CF₃ | CF(CH₃)₂ |
| E49 | NMe | 3-OCF₃ | CF(CH₃)₂ |
| E50 | NMe | 3,5-Me₂ | CF(CH₃)₂ |
| E51 | NMe | 3-OCHF₂ | CF(CH₃)₂ |
| E52 | NMe | 3-Me | CF(CH₃)₂ |
| E53 | NMe | 3-F | CF(CH₃)₂ |
| E54 | NMe | 4-F-3-Me | CF(CH₃)₂ |
| E55 | NMe | 3-Cl | CF(CH₃)₂ |
| E56 | NMe | H | CF(CH₃)₂ |
| E57 | NMe | 3-CF₃ | CHFCH₃ |
| E58 | NMe | m-OCF₃ | CHFCH₃ |
| E59 | NMe | 3,5-Me₂ | CHFCH₃ |
| E60 | NMe | 3-OCHF₂ | CHFCH₃ |
| E61 | NMe | 3-Me | CHFCH₃ |
| E62 | NMe | 3-F | CHFCH₃ |
| E63 | NMe | 4-F-3-Me | CHFCH₃ |
| E64 | NMe | 3-Cl | CHFCH₃ |
| E65 | NMe | H | CHFCH₃ |
| E66 | NMe | 3-CF₃ | CH(CH₃)₂ |
| E67 | NMe | 3-OCF₃ | CH(CH₃)₂ |
| E68 | NMe | 3,5-Me₂ | CH(CH₃)₂ |
| E69 | NMe | 3-OCHF₂ | CH(CH₃)₂ |
| E70 | NMe | 3-Me | CH(CH₃)₂ |
| E71 | NMe | 3-F | CH(CH₃)₂ |
| E72 | NMe | 4-F-3-Me | CH(CH₃)₂ |
| E73 | NMe | 3-Cl | CH(CH₃)₂ |
| E74 | NMe | H | CH(CH₃)₂ |

TABLE F

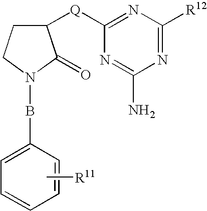

| Ex. | | | | |
|---|---|---|---|---|
| No. | Q | B | R¹¹ | R¹² |
| F1 | O | 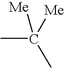 | 3-Cl | CHFCH₃ |
| F2 | O | 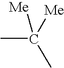 | 3-Cl | CH(CH₃)₂ |
| F3 | O | 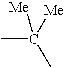 | 3-Cl | CF(CH₃)₂ |
| F4 | NH | 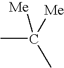 | 3-Cl | CHFCH₃ |
| F5 | NH | 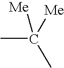 | 3-Cl | CH(CH₃)₂ |
| F6 | NH | 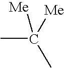 | 3-Cl | CF(CH₃)₂ |
| F7 | NMe | 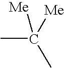 | 3-Cl | CHFCH₃ |
| F8 | NMe | 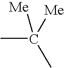 | 3-Cl | CH(CH₃)₂ |
| F9 | NMe | 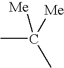 | 3-Cl | CF(CH₃)₂ |

TABLE G

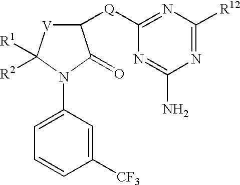

| Ex. No. | V | R¹ | R² | R¹² | Q |
|---|---|---|---|---|---|
| G1 | CH₂ | Me | Me | CF(CH₃)₂ | NH |
| G2 | CH₂ | Me | Me | CH(CH₃)₂ | NH |
| G3 | CMe₂ | H | H | CF(CH₃)₂ | NH |
| G4 | CMe₂ | H | H | CH(CH₃)₂ | NH |
| G5 | CH₂ | Me | H | CF(CH₃)₂ | NH |
| G6 | CH₂ | H | H | CH(CH₃)₂ | NH |
| G7 | S | H | H | CF(CH₃)₂ | NH |
| G8 | S | H | H | CH(CH₃)₂ | NH |
| G9 | S | H | H | CF(CH₃)₂ | NMe |
| G10 | S | H | H | CH(CH₃)₂ | NMe |
| G11 | S | H | H | CF(CH₃)₂ | O |
| G12 | S | H | H | CH(CH₃)₂ | O |
| G13 | O | H | H | CF(CH₃)₂ | NH |
| G14 | O | H | H | CH(CH₃)₂ | NH |
| G15 | O | H | H | CF(CH₃)₂ | NMe |
| G16 | O | H | H | CH(CH₃)₂ | NMe |

TABLE H

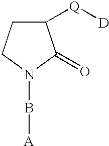

| Ex. No. | A | B | Q | D |
|---|---|---|---|---|
| H1 | 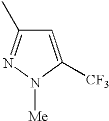 | — | O | 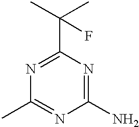 |
| H2 | 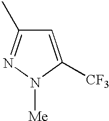 | — | NH | 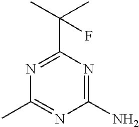 |
| H3 | 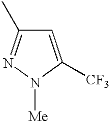 | — | NMe | 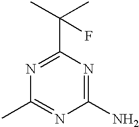 |
| H4 | 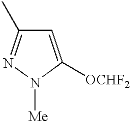 | — | O | 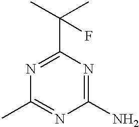 |
| H5 | 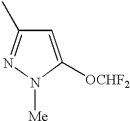 | — | NH | 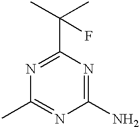 |
| H6 | 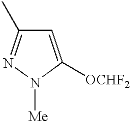 | — | NMe | 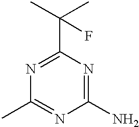 |
| H7 | 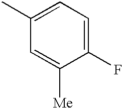 | CHMe | O | 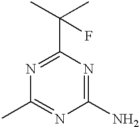 |
| H8 | 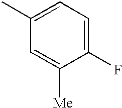 | CHMe | NH | 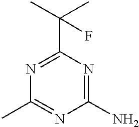 |
| H9 | 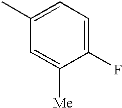 | CHMe | NMe | 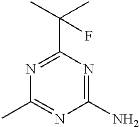 |
| H10 | 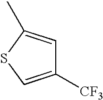 | — | S | 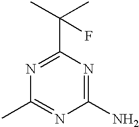 |
| H11 | 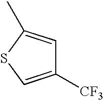 | — | O | 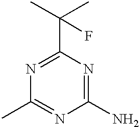 |
| H12 | 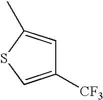 | — | NH | 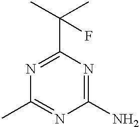 |
| H13 | 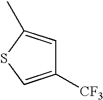 | — | NMe | 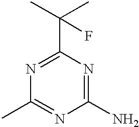 |
| H14 | 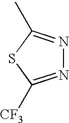 | — | S | 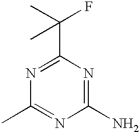 |
| H15 | 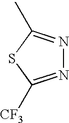 | — | O | 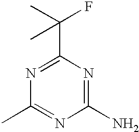 |
| H16 | 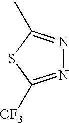 | — | NH | 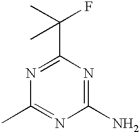 |
| H17 | 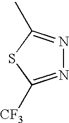 | — | NMe | 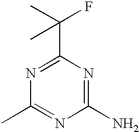 |
| H18 | 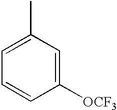 | — | N—NH₂ | 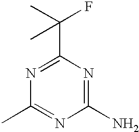 |
| H19 | 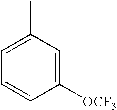 | — | 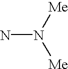 | 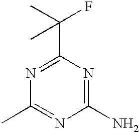 |
| H20 | 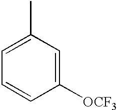 | — | N—OH | 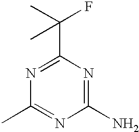 |
| H21 | 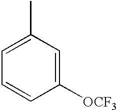 | — | N—OMe | 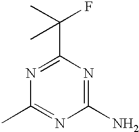 |
| H22 | 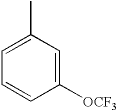 | — | CH—CN | 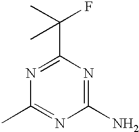 |
| H23 | 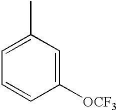 | — | CH—Me | 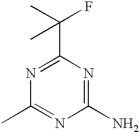 |
| H24 | 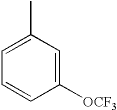 | — | CO | 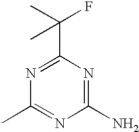 |
| H25 | 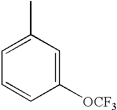 | — | C═NOH | 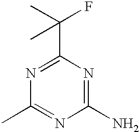 |
| H26 | 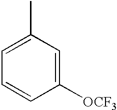 | — | C═NOMe | 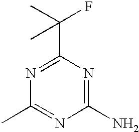 |
| H27 | 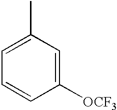 | — | 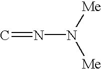 | 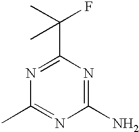 |
| H28 | 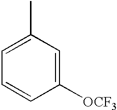 | — | SO | 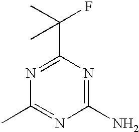 |
| H29 | 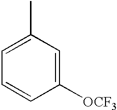 | — | SO₂ | 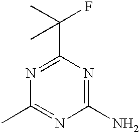 |
| H30 | 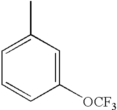 | — | CH—OH | 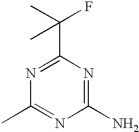 |
| H31 | 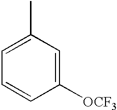 | — | CH—OMe | 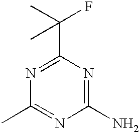 |
| H32 | 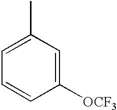 | — | CH₂ | 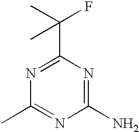 |
| H33 | 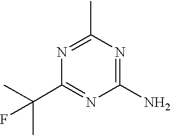 | — | O | 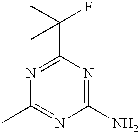 |
| H34 | 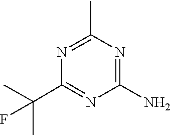 | — | NH | 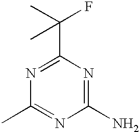 |
| H35 | 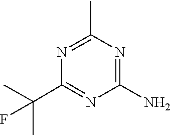 | — | NMe | 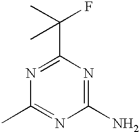 |
| H36 | 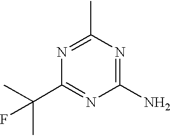 | — | O | 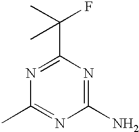 |
| H37 | 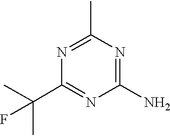 | — | NH | 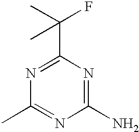 |
| H38 | 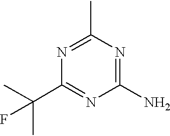 | — | NMe | 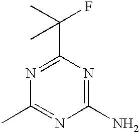 |
| H39 | 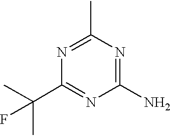 | — | O | 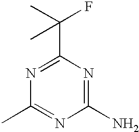 |
| H40 | 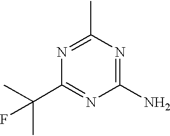 | — | NH | 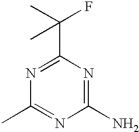 |
| H41 | 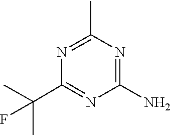 | — | NMe | 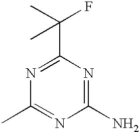 |

TABLE I
NMR data of some of the compounds listed in tables A-H. The example number
(Ex. No.) corresponds to the respective example number in the tables A-H. The
measurements were carried out using a Varian instrument (Mercury 300 MHz;
year of construction 1997).

| Ex. | |
|---|---|
| No. | ¹H-NMR δ/ppm (CDCl₃) |
| A3 | 2.10 m (1H); 2.25s (3H); 2.85 m (1H); 3.85 m (2H); 4.80 sbr (1H); 5.40 dbr (2H); |
| | 6.20 sbr (1H); 7.40 d (1H); 7.45 dd (1H); 7.85 sbr (1H); 7.90 dbr (1H) |
| A6 | 2.10 m (1H); 2.25 s (6H); 2.80 m (1H); 3.80 m (2H); 4.70 sbr (1H); 5.40 dbr (2H); |
| | 6.20 sbr (1H); 7.00 tr (1H); 7.40 m (1H); 7.50 m (1H) |
| A7 | 2.00 m (1H); 2.25s (3H); 2.80 m (1H); 3.80 s (3H); 3.80 m (2H); 4.75 sbr (1H); |
| | 5.50 dbr (2H); 6.30 sbr (1H); 6.70 dd (1H); 7.10 dbr (1H); 7.25 dd (1H); 7.40 tr (1H) |
| A8 | 2.05 m (1H); 225 s (3H); 2.80 m (1H); 3.80 m (2H); 4.80 sbr (1H); 5.40 dbr (2H); |
| | 6.20 sbr (1H); 7.00 dd (1H); 7.40 tr (1H); 7.55 dbr (1H); 7.65 sbr (1H) |
| A18 | 1.55 + 1.65 dbr (3H); 2.10 m (1H); 2.80 m (1H); 3.80 dd (2H); 4.75 m (1H); |
| | 5.10 + 5.40 q (1H); 5.80 sbr (1H); 6.30 dbr (1H); 7.10 dbr (1H); 7.30 tr (1H); 7.50 sbr |
| | (1H); 7.70 sbr (1H) |
| A25 | 1.20 d (6H); 2.10 m (1H); 2.70 sept (1H); 2.80 m (1H); 3.80s (3H); 3.80 m (2H); |
| | 4.70 m(1H); 5.30 dbr (2H); 6.00 sbr (1H); 6.30 sbr (1H); 6.70 dd (1H); 7.15 dd |
| | (1H); 7.25 tr (1H); 7.40 tr (1H) |
| A34 | 1.65 d (6H); 2.10 m (1H); 2.30 s (6H); 2.70 m (1H); 3.8Cm (2H); 4.70 sbr (2H); |
| | 6.20 sbr (1H); 6.30 sbr (1H); 6.80 s (1H); 7.20 s (2H) |
| A37 | 1.60 + 1.65 dd (3H); 2.40 m (1H); 2.75 m (1H); 3.90 m (2H); 5.25 + 5.45 dq (1H); |
| | 5.70 sbr (1H); 5.95 m (1H); 6.45 sbr (1H); 7.40 d (1H); 7.50 dd (1H); 7.90 s (1H); |
| | 7.95 d (1H) |
| A39 | 1.60 + 1.70 d (3H); 2.3Cm (1H); 2.35 s (6H); 2.70 m (1H); 3.85 m (2H); 5.25 + 5.45 |
| | dq (1H); 5.90 m (1H); 6.80 s (1H); 7.25 s (2H) |
| A42 | 1.60 + 1.65 d (3H); 2.30 m (1H); 2.70 m (1H); 3.90 m (2H); 5.25 + 5.45 dq (1H); 5.90 |
| | m (1H); 7.00 d (1H); 7.40 tr (1H); 7.60 d (1H); 7.65 s (1H) |
| A43 | 1.60 + 1.65 d (3H); 2.30 m (1H); 2.70 m (1H); 3.85 m (2H); 5.25 + 5.40 q (1H); 5.90 |
| | dtr (1H); 5.95 sbr (1H); 7.05 sbr (1H); 7.15 d (1H); 7.30 dd (1H); 7.60 dd (1H); 7.70 |
| | sbr (1H) |
| A48 | 1.20 d (6H); 2.30 m (1H); 2.30 s (6H); 2.70 m (1H); 2.80 sept (1H); 3.85 m (2H); |
| | 5.70 sbr (2H); 5.90 tr (1H); 6.80 s (1H); 7.25 s (2H) |
| A53 | 1.25 d (6H); 2.35 m (1H); 2.70 m (1H); 2.80 sept (1H); 3.90 m (2H); 5.50 sbr (2H); |
| | 5.90 tr (1H); 6.90 m (1H); 7.35 m (2H); 7.60 m (1H) |
| A54 | 1.65 d (6H); 2.40 m (1H); 2.75 m (1H); 3.95 m (2H); 5.80 sbr (1H); 5.90 tr (1H); |
| | 7.00 sbr (1H); 7.40 d (1H); 7.50 tr (1H); 7.90 s (1H); 7.90 d (1H) |
| A56 | 1.65 d (6H); 2.35 m (1H); 2.40 s (3H); 2.70 m (1H); 3.90 m (1H); 5.70 sbr (1H); |
| | 5.90 tr (1H); 6.85 sbr (1H); 7.00 d (1H); 7.25 tr (1H); 7.40 d (1H); 7.50 s (1H) |
| A57 | 1.65 d (6H); 2.30 s (6H); 2.30 m (1H); 2.70 m (1H); 3.85 m (1H); 5.80 sbr (1H); |
| | 5.85 tr (1H); 6.80 s (1H); 6.90 sbr (1H); 7.25 s (1H) |
| A59 | 1.65 d (6H); 2.35 m (1H); 2.70 m (1H); 3.80 s (3H); 3.85 m (1H); 5.80 sbr (1H); |
| | 5.90 tr (1H);6.75 dd (1H); 6.95 sbr (1H); 7.10 dd (1H); 7.25 d (1H); 7.40 dd (1H) |
| A66 | 1.60 m (3H); 2.30 m (1H); 2.50 m (1H); 3.15 s (3H); 3.85 m (2H); 5.10 m (1H); 5.20 |
| | m (1H); 7.15 d (1H); 7.30 tr (1H); 7.60 m (1H); 7.70 s (1H) |
| A77 | 1.55 + 1.65 d (3H); 2.25 m (1H); 2.40 m (1H); 3.10 s (1H); 3.80 s (1H); 3.85 m (2H); |
| | 5.30 + 5.60 tr(1H); 6.70d (1H); 7.lOd (1H); 7.3Otr(1H); 7.45s (1H) |
| E21 | 1.20 d (6H); 2.00 m (1H); 2.40 m (1H); 2.70 sbr (1H); 3.20 dd (1H); 3.40 m (1H); |
| | 3.50 m (1H); 3.70 dd (1H); 4.70 sbr (1H); 5.20 sbr (2H); 6.70 d (1H); 6.75 s (1H); |
| | 6.90 d (1H); 7.30 tr (1H) |
| E22 | 1.60 d (6H); 2.00 m (1H); 2.40 m (1H); 3.20 m (1H); 3.40 m (1H); 3.50 m (1H); |
| | 3.65 m (1H); 4.70 sbr (2H); 5.40 sbr (1H); 6.35 s (1H); 6.45 dd (1H); 6.55 d (1H); |
| | 7.20 tr (1H) |
| E23 | 1.60 dd (3H); 2.00 m (1H); 2.35 m (1H); 3.20 m (1H); 3.35 m (1H); 3.45 m (1H); |
| | 3.65 dd (1H); 4.70 sbr (2H); 5.20 dq (1H); 6.35 s (1H); 6.45 dd (1H); 6.55 d (1H); |
| | 7.20 tr (1H) |
| E24 | 1.20 d (6H); 2.00 m (1H); 2.35 m (1H); 2.70 sbr (1H); 3.20 dd (1H); 3.35 m (1H); |
| | 3.45 m (1H); 3.65 dd (1H); 4.70 sbr (1H); 5.10 sbr (2H); 6.35 s (1H); 6.45 dd (1H); |
| | 6.55 d (1H); 7.20 tr (1H) |
| E25 | 1.60 d (6H); 2.00 m (1H); 2.25 s (6H); 2.30 m (1H); 3.20 m (1H); 3.35 m (1H); 3.45 |
| | m (1H); 3.60 m (1H); 4.70 sbr (1H); 5.40 sbr (2H); 6.20 s (2H); 6.40 s (1H) |
| E26 | 1.60 dd (3H); 2.00 m (1H); 2.25 s (6H); 2.35 m (1H); 3.20 m (1H); 3.30 m (1H); |
| | 3.45 m (1H); 3.60 m (1H); 4.70 sbr (1H); 5.20 dq (1H); 5.40 sbr (2H); 6.20 s (2H); |
| | 6.40 s (1H) |
| E27 | 1.20 d (6H); 2.00 m (1H); 2.25s (6H); 2.35 m (1H); 3.20 dd (1H); 3.35 m (1H); |
| | 3.45 m (1H); 3.60 dd (1H); 4.70 sbr (1H); 5.20 sbr (2H); 6.20 s (2H); 6.40 s (1H) |
| F1 | 1.60 d (3H); 1.65 d (3H); 1.75s (3H); 1.80s (3H); 2.15 m (1H); 2.5Cm (2H); 3.40 |
| | m (2H); 5.25 dq (1H); 5.40 dq (1H); 5.75 dtr (1H); 5.80 sbr (1H); 6.90 sbr (1H); |
| | 7.25 m (4H) |
| F2 | 1.20 d (6H); 1.75s (3H); 1.80s (3H); 2.10 m (1H); 2.50 m (1H); 2.80 sept (1H); |
| | 3.40 m (2H); 5.60 sbr (2H); 5.75 tr (1H); 7.25 m (4H) |
| F3 | 1.65 d (6H); 1.75 s (3H); 1.80 s (3H); 2.20 m (1H); 2.50 m (1H); 3.45 m (2H); 5.75 |
| | tr (1H); 5.75 sbr (1H); 7.00 sbr (1H); 7.25 m (4H) |

Abbreviations used:
ppm = parts per million
CDCl₃ = deuterated trichioromethane
s = singlet
sbr = broad singlet
d = doublet
dbr = broad doublet
dd = doublet of a doublet
tr = triplet
q = quartet
sept = septet
m = multiplet

TABLE J
Melting points of some of the compounds listed in tables A-H. The
example number corresponds to the example number in tables A-H.

| Ex. No. | mp/° C. |
|---|---|
| A1 | 133 |
| A2 | 146 |
| A4 | 210 |
| A5 | 210 |
| A9 | 155 |
| A10 | 138 |
| A11 | 223 |
| A12 | 170 |
| A13 | 185 |
| A14 | 132 |
| A15 | 178 |
| A16 | 133 |
| A17 | 110 |
| A19 | 190 |
| A20 | 210 |
| A21 | 77 |
| A22 | 73 |
| A23 | 99 |
| A24 | 71 |
| A26 | 85 |
| A27 | 176 |
| A28 | 123 |
| A29 | 187 |
| A30 | 93 |
| A31 | 117 |
| A32 | 118 |
| A33 | 101 |
| A35 | 230 |
| A36 | 113 |
| A38 | 170 |
| A40 | 163 |
| A41 | 154 |
| A44 | 176 |
| A45 | 146 |
| A46 | 154 |
| A47 | 146 |
| A49 | 164 |
| A50 | 150 |
| A51 | 141 |
| A52 | 54 |
| A55 | 77 |
| A58 | 189 |
| A60 | 121 |
| A61 | 170 |
| A62 | 179 |
| A63 | 107 |
| A64 | 182 |
| A65 | 181 |
| A67 | 196 |
| A68 | 206 |
| A69 | 184 |
| A70 | 153 |
| A71 | 125 |
| A72 | 211 |
| A73 | 182 |
| A74 | 216 |
| A75 | 162 |
| A76 | 210 |
| A78 | 169 |
| A79 | 211 |
| A80 | 194 |
| A81 | 181 |
| A82 | 204 |
| A83 | 208 |
| A84 | 208 |
| E7 | 83 |
| E8 | 67 |
| E9 | 173 |
| E10 | 97 |
| E11 | 72 |
| E12 | 155 |
| E13 | 194 |
| E14 | 175 |
| E15 | 178 |
| E16 | 148 |
| E17 | 171 |
| E18 | 172 |
| E19 | 155 |
| E20 | 57 |
| E48 | 165 |
| E49 | 62 |
| E50 | 177 |
| E55 | 195 |
| E57 | 147 |
| E58 | 71 |
| E59 | 179 |
| E64 | 177 |
| E66 | 164 |
| E67 | 142 |
| E68 | 163 |
| E73 | 180 |

Abbreviations used:
mp = melting point
° C. = degrees Celsius

### C. Formulation Examples

a) A dust is obtained by mixing 10 parts by weight of a compound of the formula (I) and 90 parts by weight of talc as inert substance and comminuting the mixture in a hammer mill.

b) A wettable powder which is readily dispersible in water is obtained by mixing 25 parts by weight of a compound of the formula (I), 64 parts by weight of kaolin-containing quartz as inert substance, 10 parts by weight of potassium lignosulfonate and 1 part by weight of sodium oleoylmethyltaurinate as wetter and dispersant and grinding the mixture in a pinned-disk mill.

c) A dispersion concentrate which is readily dispersible in water is obtained by mixing 20 parts by weight of a compound of the formula (I) with 6 parts by weight of alkylphenol polyglycol ether ®Triton X 207), 3 parts by weight of isotridecanol polyglycol ether (8 EO) and 71 parts by weight of paraffinic mineral oil (boiling range for example approx. 255 to above 277° C.) and grinding the mixture in a ball mill to a fineness of below 5 microns.

d) An emulsifiable concentrate is obtained from 15 parts by weight of a compound of the formula (I), 75 parts by weight of cyclohexanone as the solvent and 10 parts by weight of ethoxylated nonylphenol as the emulsifier.

e) Water-dispersible granules are obtained by mixing

75 parts by weight of a compound of the formula (I),

10 parts by weight of calcium lignosulfonate,

5 parts by weight of sodium lauryl sulfate,

3 parts by weight of polyvinyl alcohol and

7 parts by weight of kaolin

grinding the mixture on a pinned-disk mill and granulating the powder in a fluidized bed by spraying on water as the granulation liquid.

f) Water-dispersible granules are also obtained by homogenizing and precomminuting, on a colloid mill,

25 parts by weight of a compound of the formula (I),

5 parts by weight of sodium 2,2′-dinaphthylmethane-6,6′-disulfonate

2 parts by weight of sodium oleoylmethyltaurinate,

1 part by weight of polyvinyl alcohol,

17 parts by weight of calcium carbonate and

50 parts by weight of water,

subsequently grinding the mixture in a bead mill and atomizing and drying the resulting suspension in a spray tower by means of a single-fluid nozzle.

### D. Biological Examples

Dl. Pre-Emergence Effect on Weeds

Seeds or rhizome pieces of monocotyledonous and dicotyledonous weed plants are placed in sandy loam soil in plastic pots and covered with soil. The compounds according to the invention which are formulated in the form of wettable powders or emulsion concentrates are then applied to the surface of the soil cover in the form of aqueous suspensions or emulsions at an application rate of 600 to 800 l of water/ha (converted), in various dosages.

After the treatment, the pots are placed in a greenhouse and kept under good growth conditions for the weeds. After the test plants have emerged, the damage to the plants or the negative effect on the emergence is scored visually after a test period of 3 to 4 weeks by comparison with untreated controls. As shown by the test results, compounds according to the invention have good herbicidal pre-emergence activity against a broad spectrum of weed grasses and broad-leafed weeds. For example, in the test the compounds of Example Nos. A1, A2, A3, A4, A5, A6, A7, A8, A9, A10, A11, A12, A13, A14, Al5, A16, A17, A18, A19, A20, A21, A22, A23, A24, A25, A26, A27, A28, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A80, A81, A82, A83, A84, E7, E8, E9, E10, E11, E12, E13, E14, E15, E16, E17, E18, E19, E20, E21, E22, E23, E24, E25, E26, E27, E48, E49, E50, E55, E57, E58, E59, E64, E66, E67, E68, E73, F1, F2 and F3 (see Tables A-H) have very good herbicidal activity pre-emergence against weed plants such as Digitaria sanguinalis, Echinochloa crus-galli, Setaria faberi, Setaria viridis, Sorghum halepense, Abutilon theophrasti, Amaranthum retroflexus, Ambrosia artemisifolia, Chenopodium album, Datura stramonium, Kochia scoparia, Pharbitis purpurea, Sido spinosa, Solanum nigrum und Xanthium strumarium at an application rate of 1 kg or less of active substance per hectare.

D2. Post-Emergence Effect on Weeds

Seeds or rhizome pieces of monocotyledonous and dicotyledonous weeds are placed in sandy loam soil in plastic pots, covered with soil and grown in a greenhouse under good growth conditions. Three weeks after sowing, the test plants are treated at the three-leaf stage. The compounds according to the invention which were formulated as wettable powders or emulsion concentrates are sprayed, at various dosages, onto the green parts of the plants at an application rate of 600 to 800 l of water/ha (converted). After the test plants have remained in the greenhouse for about 3 to 4 weeks under optimum growth conditions, the effect of the preparations is scored visually by comparison with untreated controls. The agents according to the invention also have good herbicidal activity post-emergence against a broad spectrum of economically important weed grasses and broad-leafed weeds. For example, the compounds of Example Nos. A1, A2, A3, A4, A5, A6, A7, A8, A9, A10, A11, A12, A13, A14, A15, A16, A17, A18, A19, A20, A21, A22, A23, A24, A25, A26, A27, A28, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A80, A81, A82, A83, A84, E7, E8, E9, E10, E11, E12, E13, E14, E15, E16, E17, E18, E19, E20, E21, E22, E23, E24, E25, E26, E27, E48, E49, E50, E55, E57, E58, E59, E64, E66, E67, E68, E73, F1, F2 and F3 (see Tables A-H) have very good herbicidal activity post-emergence against harmful plants such as Digitaria sanguinalis, Echinochloa crus-galli, Setaria faberi, Setaria viridis, Sorghum halepense, Abutilon theophrasti, Amaranthum retroflexus, Ambrosia artemisifolia, Chenopodium album, Datura stramonium, Kochia scoparia, Pharbitis purpurea, Sido spinosa, Solanum nigrum and Xanthium strumarium at an application rate of 1 kg and less of active substance per hectare.

3. Action on Weed Plants in Rice

Transplanted and sown rice and also typical rice weeds (gramineous and broad-leaves) are cultivated in closed plastic pots in a greenhouse to the three-leaf stage (Echinochloa crus-galli 1.5-leaf) under paddy rice conditions (dammed height of water: 2-3 cm). This is followed by treatment with the novel compounds. For this purpose, the formulated active compounds are suspended, dissolved or emulsified in water and applied by pouring them into the dammed water around the test plants in different dosages. After this treatment, the test plants are set up in a greenhouse under optimum growth conditions and are maintained in this way throughout the test period.

About three weeks after application, evaluation is made by visual scoring of the damage to the plants by comparison with untreated controls. The compounds according to the invention show very good herbicidal activity against weed plants.

4. Tolerance by Crop Plants

In further greenhouse experiments, seeds of a substantial number of crop plants and weeds are placed in sandy loam soil and covered with soil. Some of the pots are treated immediately as described under Section 1, and the remaining pots are placed in the greenhouse until the plants have developed two to three true leaves and then sprayed with various dosages of the substances of the formula (I) according to the invention, as described under Section 2. Visual scoring four to five weeks after the application and after the plants have been in the greenhouse reveals that compounds according to the invention leave dicotyledonous crops such as soya, cotton, oilseed rape, sugar beet and potatoes unharmed even when high dosages of active ingredient are used in the pre- and post-emergence method. Moreover, some substances also spare Gramineae crops such as barley, wheat, rye, sorghum species, corn or rice. Some of the compounds of the formula (I) have high selectivity, and they are therefore suitable for controlling unwanted vegetation in agricultural crops.

What is claimed is:

1. A compound of the formula (I) or salt thereof, 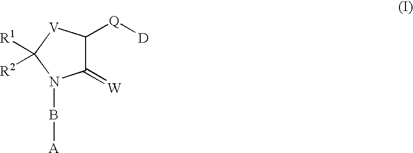
in which
A is a radical as defined under (a1) to (a5), where
(a1) is unsubstituted aryl having 6-18 carbon atoms or unsubstituted heteroaryl or heterocyclyl having from 2 to 14 carbon atoms, at least one ring containing one or more identical or different ring heteroatoms from the group N, O, and S,
(a2) is a radical as defined under (a1) which is further substituted by one or more fused-on nonaromatic carbocyclic rings having in each case from 4 to 7 ring atoms or heterocyclic rings having in each case from 4 to 7 ring atoms and one or more ring heteroatoms from the group N, O, and S,
(a3) is a radical as defined under (a1) or (a2) which is further substituted,
(a4) is (C₁-C₆)-alkyl, (C₃-C₉)-cycloalkyl, (C₂-C₆)-alkenyl, (C₅-C₉)-cycloalkenyl or (C₂-C₆)-alkynyl,
(a5) is one of the hydrocarbon radicals defined under (a4) which further carries one or more identical or different radicals from the group of the radicals of subgroups (a5.1) and (a5.2), where
subgroup (a5.1) is composed of the radicals halogen, amino, hydroxyl, nitro, cyano, mercapto, carboxyl, carbamoyl, SF₅, aminosulfonyl, (C₃-C₉)-cycloalkyl, (C₅-C₉)-cycloalkenyl, mono-(C₁-C₆)-alkylamino, di-((C₁-C₆)-alkyl)-amino, N-((C₁-C₆)-alkanoyl)-amino, (C₁-C₆)-alkoxy, (C₂-C₆)-alkenyloxy, (C₂-C₆)-alkynyloxy, (C₃-C₉)-cycloalkoxy, (C₅-C₉)-cycloalkenyloxy, ((C₁-C₆)-alkoxy)-carbonyl, (C₁-C₆)-alkanoyl, (C₁-C₆)-alkylthio, (C₃-C₉)-cycloalkylthio, (C₂-C₆)-alkenylthio, (C₅-C₉)-cycloalkenylthio, (C₂-C₆)-alkynylthio, (C₁-C₆)-alkynylsulfinyl, (C₁-C₆)-alkylsulfonyl, mono-((C₁-C₆)-alkyl)-aminosulfonyl, di-((C₁-C₆)-alkyl)-aminosulfonyl, N-((C₁-C₆)-alkyl)-aminocarbonyl, N, N-di-((C₁-C₆)-alkyl)-aminocarbonyl, N-((C₁-C₆)-alkanoyl)-aminocarbonyl, N-((C₁-C₆)-alkanoyl)-N-((C₁-C₆)-alkyl)-aminocarbonyl and, in the case of cyclic radicals, additionally (C₁-C₄)-alkyl, (C₂-C₆)-alkenyl, (C₂-C₆)-alkynyl, (C₁-C₄)-alkoxy-(C₁-C₄)-alkyl, ((C₁-C₄)-alkoxy)-carbonyl-(C₁-C₄)-alkyl, (C₁-C₄)-alkylthio)-(C₁-C₄)-alkyl, mono-((C₁-C₄)-alkyl)-amino-(C₁-C₄)-alkyl and di-((C₁-C₄)-alkyl)-amino-(C₁-C₄)-alkyl, each of the above hydrocarbon substituents (a5.1) being unsubstituted or substituted by one or more identical or different radicals from the group consisting of halogen, nitro, cyano, thiocyanato, (C₁-C₄)-alkoxy, (C₁-C₄)-haloalkoxy and (C₁-C₄)-alkylthio and, in the case of cyclic radicals, additionally (C₁-C₄)-alkyl and halo-(C₁-C₄)-alkyl, and
subgroup (a5.2) is composed of the radicals aryl, aryloxy, arylthio, heteroaryloxy and heteroarylthio, each of the last-mentioned 6 radicals being unsubstituted or substituted by one or more identical or different radicals from the group [=substituent group (a5.2.1)] consisting of halogen, amino, hydroxyl, nitro, cyano, mercapto, carboxyl, carbamoyl, aminosulfonyl, (C₁-C₄)-alkyl, (C₁-C₄)-haloalkyl, ((C₁-C₄)-alkoxy)-(C₁-C₄)-alkyl, ((C₁-C₄)-alkoxy)-carbonyl-(C₁-C₄)-alkyl, ((C₁-C₄)-alkylthio)-(C₁-C₄)-alkyl, mono-((C₁-C₄)-alkyl)-amino-(C₁-C₄)-alkyl and di-(C₁-C₄)-alkyl)-amino-(C₁-C₄)-alkyl, (C₂-C₆)-cycloalkyl, (C₂-C₄)-alkenyl, (C₅-C₉)-cycloalkenyl, (C₂-C₆)-alkynyl, mono-((C₁-C₄)-alkyl)-amino, di-((C₁-C₄)-alkyl)-amino, N-((C₁-C₆)-alkanoyl)-amino, N-((C₁-C₆)-alkanoyl)-amino, N-((C₁-C₆)-alkanoyl)-N-((C₁-C₄)-alkyl)-amino, (C₁-C₆)-alkoxy, (C₁-C₆)-alkoxy-[poly-(C₁-C₄)-alkylenoxy)]-, hydroxy-[poly-(C₁-C₄)-alkylenoxy)]—(C₃-C₆)-cycloalkylenoxy, (C₂-C₆)-haloalkenyloxy, (C₂-C₆)-alkynyloxy, (C₂-C₆)-haloalkynyloxy, ((C₁-C₄)-alkoxy)-carbonyl, (C₁-C₆)-alkanoyl, (C₁-C₄)-alkylthio, (C₃-C₆)-cycloalkylthio, (C₂-C₄)-alkenylthio, (C₅-C₉)-cycloalkenylthio, (C₂-C₄)-alkynylthio, (C₁-C₄)-alkylsulfinyl, (C₁-C₄)-alkylsulfonyl, mono-(C₁-C₄)-alkylaminosulfonyl, di-((C₁-C₄)-alkyl)-aminosulfonyl, N-((C₁-C₄)-alkyl)-aminocarbonyl, N,N-di-((C₁-C₄)-alkyl)-aminocarbonyl, N-((C₁-C₄)-alkanoyl)-aminocarbonyl and N-((C₁-C₄)-alkanoyl)-N-((C₁-C₄)-alkyl)-aminocarbonyl, each of the hydrocarbon substituents from the above substituent subgroup (a5.2.1) being unsubstituted or substituted by one or more identical or different radicals from the group consisting of halogen, nitro, cyano, thiocyanato, (C₁-C₄)-alkoxy, (C₁-C₄)-haloalkoxy and (C₁-C₄)-alkylthio and, in the case of cyclic radicals, additionally (C₁-C₄)-alkyl and halo-(C₁-C₄)-alkyl,
B is a direct bond or (C₁-C₄)-alkylene, (C₂-C₄)-alkenylene, (C₂-C₄)-alkynylene, it being possible for individual carbon atoms to have been replaced by nitrogen atoms,
W is O, S or H₂,
V is a divalent group of the formula CH₂, S, O, or CHR³ or CR³R⁴, where
R³ is selected from the group consisting of
(C₁-C₆)-alkyl, (C₂-C₆)-alkenyl, (C₂-C₆)-alkynyl, (C₃-C₆)-cycloalkyl, (C₃-C₆)-cycloalkenyl, mono-(C₁-C₆)-alkylamino, di-((C₁-C₆)-alkyl)-amino, N-(C₁-C₆)-alkanoyl)-amino, (C₁-C₆)-alkoxy, (C₂-C₆)-alkenyloxy, (C₂-C₆)-alkynyloxy, (C₃-C₉)-cycloalkoxy, (C₅-C₉)-cycloalkenyloxy, (C₁-C₆)-alkylthio, (C₃-C₉)-cycloalkylthio, (C₂-C₆)-alkenylthio, (C₅-C₉)-cycloalkenylthio, (C₂-C₆)-alkynylthio, (C₁-C₆)-alkanoyl, (C₂-C₆)-alkenylcarbonyl, (C₂-C₆)-alkynylcarbonyl, arylcarbonyl, (C₁-C₆)-alkoxycarbonyl, (C₂-C₆)-alkenyloxycarbonyl, (C₂-C₆)-alkynoxycarbonyl, aryloxycarbonyl, (C₁-C₆)-alkylsulfinyl and (C₁-C₆)-alkylsulfonyl, each of the above radicals indicated for R³ being unsubstituted or substituted by halogen or cyano, and
R⁴ independently of R³ being one of the radicals defined under R³,
R¹ and R² independently of one another are each H, amino, (C₁-C₆)-alkyl, (C₂-C₆)-alkenylcarbonyl, (C₂-C₆)-alkynylcarbonyl, mono-(C₁-C₆)-alkylamino, (C₃-C₆)-cycloalkyl, (C₁-C₆)-alkoxy, (C₃-C₆)-cycloalkoxy, (C₁-C₆)-alkanoyl, (C₁-C₆)-alkenylcarbonyl, (C₂-C₆)-alkynylcarbonyl, (C₁-C₆)-alkoxycarbonyl, di-((C₁-C₆)-alkyl)-amino-(C₁-C₆)-alkyl, di-((C₁-C₆)-alkyl)-amino-(C₁-C₆)-alkenyl, di-((C₁-C₆)-alkyl)-aminocarbonyl, each of the last-mentioned 14 radicals being unsubstituted or substituted by one or more radicals from the group consisting of halogen and cyano,
Q is a divalent group of the formula O, S, SO, SO₂, NR⁵, CR⁶R⁷, CR⁶OR⁷, CO, CS or C═N—R′, in which R′ is H or (C₁-C₄)-alkyl, and in the formulae
R⁶ and R⁷ independently of one another are hydrogen, (C₁-C₄)-alkyl, (C₁-C₄)-alkenyl, (C₃-C₆)-cycloalkyl, (C₃-C₆)-cycloalkenyl or cyano and R⁵ is hydrogen, hydroxyl, formyl, NR⁸R⁹, (C₁-C₄)-alkyl, (C₂-C₄)-alkenyl, (C₂-C₄)-alkynyl, (C₃-C₆)-cycloalkyl, (C₃-C₆)-cycloalkenyl, (C₁-C₄)-alkyloxy, (C₂-C₄)-alkenyloxy or (C₂-C₄)-alkynyloxy, each of the last-mentioned hydrocarbon radicals for R⁵ being unsubstituted or substituted by one or more substituents from the group consisting of OR⁸, COR⁸, COOR⁸, OCOR⁸, CN, halogen, S(O)ₚR⁸ [where p=0, 1, or 2], NR⁸R⁹, NO₂, NR⁸COR⁹, NR⁸CONR⁹R¹⁰, CONR⁸R⁹ and heterocyclyl, and
each of the radicals R⁸, R⁹ and R¹⁰ independently of one another being hydrogen, (C₁-C₄)-alkyl, (C₂-C₄)-alkenyl or (C₂-C₄)-alkynyl, each of the last-mentioned 3 radicals being unsubstituted or substituted by halogen, and
D is aryl if Q is selected from the group O, S, SO, SO₂ or NR⁵, or is heteroaryl having 3-5 carbon atoms and 1, 2, or 3 identical or different ring heteroatoms selected from the group N, O, and S, if Q is selected from the group O, S, SO, SO₂, NR⁵, CR⁶R⁷, CR⁶OR⁷, CO, CS or C═N—R′, the aryl or the heteroaryl being unsubstituted or substituted.

2. A compound of the formula (I) or salt thereof as claimed in claim 1, wherein D is a 1,3,5-triazine, a diazabenzene or a phenyl which carries one or more identical or different substituents.

3. A compound of the formula (I) or salt thereof as claimed in claim 1, in which D possesses one or more identical or different radicals from the group consisting of amino, hydroxyl, halogen, nitro, cyano, mercapto, thiocyanato, carboxyl, carbamoyl, SF₅, aminosulfonyl, (C₁-C₆)-alkyl, (C₃-C₉)-cycloalkyl, (C₂-C₆)-alkenyl, (C₅-C₉)-cycloalkenyl, (C₂-C₆)-alkynyl, mono-((C₁-C₆)-alkyl)-amino, di-((C₁-C₆)-alkyl)-amino, ((C₃-C₆)-cycloalkyl)-amino, N-((C₁-C₆)-alkanoyl)-amino, (C₁-C₆)-alkoxy, (C₂-C₆)-alkenyloxy, (C₂-C₆)-alkynyloxy, (C₃-C₉)-cycloalkoxy, (C₅-C₉)-cycloalkenyloxy, ((C₁-C₆)-alkoxy)-carbonyl, ((C₂-C₆)-alkenyloxy)-carbonyl, ((C₂-C₆)-alkynyloxy)-carbonyl, aryloxycarbonyl, (C₁-C₆)-alkanoyl, ((C₂-C₆)-alkenyl)-carbonyl, ((C₂-C₆)-alkynyl)-carbonyl, arylcarbonyl, (C₁-C₆)-alkylthio, (C₃-C₉)-cycloalkylthio, (C₂-C₆)-alkenylthio, (C₅-C₉)-cycloalkenylthio, (C₂-C₆)-alkynylthio, (C₁-C₆)-alkylsulfinyl, (C₁-C₆)-alkylsulfonyl, mono-((C₁-C₆)-alkyl)-aminosulfonyl, di-((C₁-C₆)-alkyl)-aminosulfonyl, N-((C₁-C₆)-alkyl)-aminocarbonyl, N,N-di-((C₁-C₆)-alkyl)-aminocarbonyl, N-((C₁-C₆)-alkanoyl)-aminocarbonyl, N-((C₁-C₆)-alkanoyl)-N-((C₁-C₆)-alkyl)-aminocarbonyl, aryl, aryloxy, benzyl, benzyloxy, benzylthio, arylthio, tri-((C₁-C₆)-alkyl)-silyl, (C₁-C₆)-alkanoyloxy, (C₁-C₆)-alkylsulfonyloxy, (C₁-C₆)-alkoxycarbonyloxy, (C₂-C₆)-alkenyloxycarbonyloxy, (C₂-C₆)-alkynyloxycarbonyloxy, N-((C₁-C₆)-alkyl)-aminocarbonyloxy and N,N,-di-((C₁-C₆)-alkyl)-aminocarbonyloxy, each of the above hydrocarbon substituents being unsubstituted or substituted by one or more identical or different radicals from the group consisting of amino, hydroxyl, halogen, nitro, cyano, mercapto, carboxyl, carbamoyl, aminosulfonyl, (C₃-C₆)-cycloalkyl, (C₂-C₄)-alkenyl, (C₅-C₉)-cycloalkenyl, (C₂-C₆)-alkynyl, mono-(C₁-C₄)-alkylamino, di-((C₁-C₄)-alkyl)-amino, N-((C₁-C₆)-alkanoyl)-amino, N-((C₁-C₆)-alkanoyl)-N-((C₁-C₄)-alkyl)-amino, (C₁-C₆)-alkoxy, (C₁-C₆)-alkoxy-(C₁-C₆)-alkoxy, (C₁-C₆)-alkoxy-poly-[(C₁-C₄)-alkylenoxy)]-, hydroxy-(C₁-C₆)-alkoxy, hydroxy-[poly-(C₁-C₄)-alkylenoxy)]-, (C₃-C₆)-cycloalkoxy, (C₁-C₆)-haloalkoxy, (C₂-C₆)-alkenyloxy, (C₅-C₉)-cycloalkenyloxy, (C₂-C₆)-haloalkenyloxy, (C₂-C₆)-alkynyloxy, (C₂-C₆)-haloalkynyloxy, (C₁-C₄)-alkoxycarbonyl, aryloxycarbonyl, (C₁-C₆)-alkanoyl, arylcarbonyl, (C₁-C₄)-alkylthio, (C₃-C₆)-cycloalkylthio, (C₂-C₄)-alkenylthio, (C₅-C₉)-cycloalkenylthio, (C₂-C₄)-alkynylthio, (C₁-C₄)-alkylsulfinyl, (C₁-C₄)-alkylsulfonyl, mono-((C₁-C₄)-alkyl)-aminosulfonyl, di-((C₁-C₄)-alkyl)-aminosulfonyl, N-((C₁-C₄)-alkyl)-amino-carbonyl, N,N,-di-((C₁-C₄)-alkyl)-aminocarbonyl, N-((C₁-C₄)-alkanoyl)-aminocarbonyl and N-((C₁-C₄)-alkanoyl)-N-((C₁-C₄)-alkyl)-aminocarbonyl and, in the case of cyclic radicals, additionally (C₁-C₄)-alkyl, (C₁-C₄)-haloalkyl, (C₂-C₄)-alkenyl, (C₂-C₄)-alkynyl, (C₂-C₆)-haloalkenyl, (C₂-C₆)-haloalkynyl, (C₂-C₄)-alkoxy)-(C₁-C₄)-alkyl, (C₁-C₄)-alkoxycarbonyl-(C₁-C₄)-alkyl, (C₁-C₄)-alkylthio-(C₁-C₄)-alkyl, mono-((C₁-C₄)-alkyl)-amino-(C₁-C₄)-alkyl and di-((C₁-C₄)-alkyl)-amino-(C₁-C₄)-alkyl.

4. A compound of the formula (I) or salt thereof as claimed in claim 1, in which A is a substituted or unsubstituted monocyclic, bicyclic or tricyclic aromatic system comprising five- or six-membered carbocyclic rings.

5. A compound of the formula (I) or salt thereof as claimed in claim 1, wherein A is an unsubstituted or substituted phenyl.

6. A compound of the formula (I) or salt thereof as claimed in claim 1, in which A is a substituted five- or six-membered aromatic ring system containing one or more identical or different heteroatoms from the group consisting of nitrogen, oxygen, and sulfur.

7. A compound of the formula (I) or salt thereof as claimed in claim 1, in which A is a pyrazolidine which carries one or more identical or different substituents.

8. A compound of the formula (I) or salt thereof as claimed in claim 4, wherein the constituents of A are selected from the group consisting of methyl, methoxy, ethoxy, fluorine, chlorine, bromine, iodine, trifluoromethyl, trifluoromethoxy, difluoromethyl, difluoromethoxy, trichloromethyl, trichloromethoxy, dichloromethoxy, fluoromethoxy, chloromethoxy, tetrafluoroethoxy, trifluoroethoxy, trichloroethoxy, difluoroethoxy and dichloroethoxy.

9. A compound of the formula (I) or salt thereof as claimed in claim 1, where B is selected from the group of the formula —CH═CH—, —C≡C—, —N═CH—, —NH—CH₂—, and where each of said divalent radicals is unsubstituted or substituted by one or more radicals from the group consisting of halogen, nitrogen, and cyano.

10. A process for preparing a compound of the formula (I), as defined in claim 1, or a salt thereof, which comprises
(a) where Q is a radical selected from the group of O, S or NR⁵, reacting a compound of the formula Z-D, in which Z is an exchangeable radical or a leaving group, with an appropriate compound of the formula (II) or an acid addition salt thereof, the radicals R¹, R², R⁵, A, B, V, W and D in the formulae (II) and Z-D being as defined in formula (I), 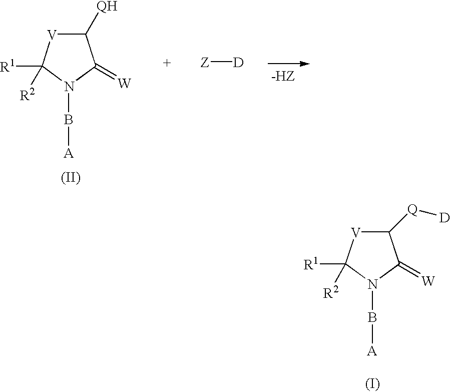
or
b) where Q is a radical selected from the group of O, S and NR⁵, reacting a compound of the formula (III) in which Z is an exchangeable radical or leaving group with an appropriate compound of the formula HQ-D or an acid addition salt thereof, the radicals R¹, R², R⁵, A, B, V, W and D in the formulae (III) and HQ-D being as defined for formula (I) 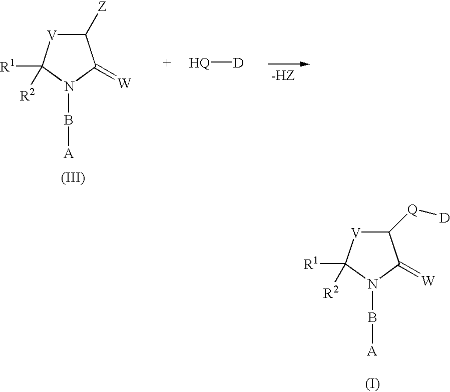
or
c) where Q in formula (I) is a radical of the formula CHCN (=compound of the formula (I′)), reacting a compound of the formula Z-D in which Z is an exchangeable radical or leaving group with an appropriate compound of the formula (IV), the radicals R¹, R², A, B, V, W, and D in the formulae (IV) and Z-D being as defined in formula (I), 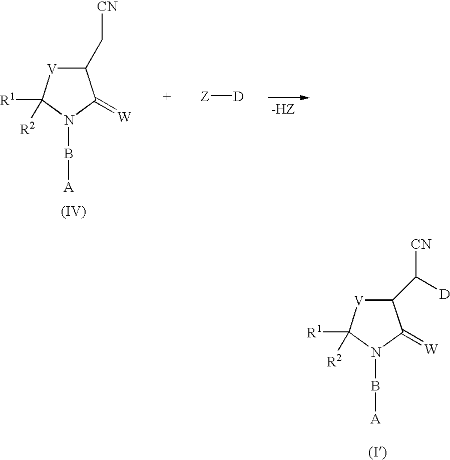
or
d) alternatively to the process described under c), where Q is a radical of the formula CHCN, reacting a compound of the formula (III) in which Z is an exchangeable radical or leaving group with an appropriate compound of the formula NC—CH₂-D, the radicals R¹, R², A, B, V, W, and D in the formula (III) and in the formula NC—CH₂-D being as defined for formula (I), 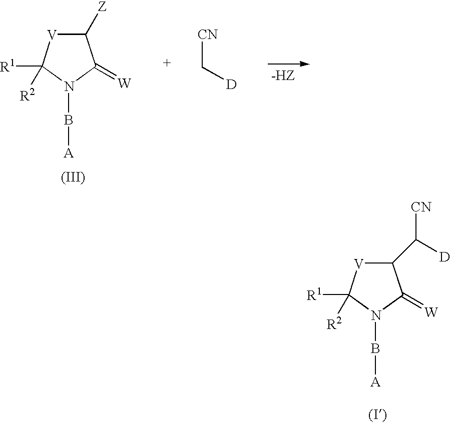
or
e) where D in formula (I) is a radical of the formula (IX) and Q=NR⁵ (=compounds of the formula (III′)), reacting compounds of the formula (V) or an acid addition salt with compounds of the formula (VI), 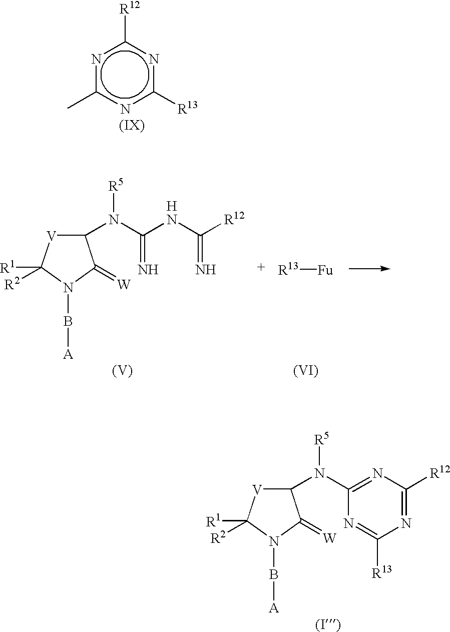
in which Fu is a functional group from the group consisting of carboxylate, orthocarboxylate, carbonyl chloride, carboxamide, carboxylic anhydride, and trichloromethyl, the radicals R¹, R², R⁵, A, B, V, and W being as defined in formula (I) and R¹² and R¹³ being as defined in formula (IX).

11. A compound of the formula (V) as defined in claim 10.

12. A process for preparing a compound of the formula (V) from a compound of the formula (VII) and/or an acid adduct thereof by reaction with a cyanoguanide (“dicyanodiamide”) of the formula (VII).

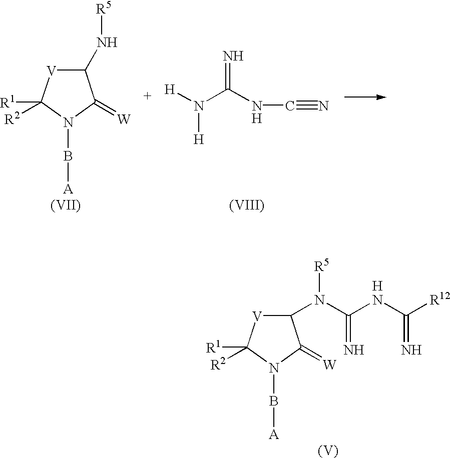

13. A herbicidal or plant growth regulating composition comprising one or more compounds of the formula (I) or salts thereof as claimed in claim 1 and customary plant protection formulating auxiliaries.

14. A method of controlling weed plants or of regulating plant growth, which comprises applying an effective amount of one or more compounds of the formula (I) or salts thereof as claimed in claim 1 to the plants, parts of plants, seeds of plants, or the cultivation area.

15. The use of a compound of the formula (I) or salt thereof as claimed in claim 1 as a herbicide or plant growth regulator.
